SEQUENCE LISTING (1000 sequences)

SEQ ID NO: 1 - RNA, 22 nt, Homo sapiens
uaacacugucugguaaagaugg

SEQ ID NO: 2 - RNA, 22 nt, Homo sapiens
ugagaacugaauuccauaggcu

SEQ ID NO: 3 - RNA, 23 nt, Homo sapiens
ugugcaaaucuaugcaaaacuga

SEQ ID NO: 4 - RNA, 22 nt, Homo sapiens
uuaaugcuaaucgugauagggg

SEQ ID NO: 5 - RNA, 23 nt, Homo sapiens
uguaguguuuccuacuuuaugga

SEQ ID NO: 6 - RNA, 22 nt, Homo sapiens
uggcucaguucagcaggaacag

SEQ ID NO: 7 - RNA, 20 nt, Homo sapiens
cauaaaguagaaagcacuac

SEQ ID NO: 8 - RNA, 23 nt, Homo sapiens
ugugcaaauccaugcaaaacuga

SEQ ID NO: 9 - RNA, 23 nt, Homo sapiens
uaaggugcaucuagugcagauag

SEQ ID NO: 10 - RNA, 23 nt, Homo sapiens
caaagugcuuacagugcagguag

SEQ ID NO: 11 - RNA, 22 nt, Homo sapiens
ugucaguuugucaaauacccca

SEQ ID NO: 12 - RNA, 60 nt, Homo sapiens
ccuggaugaugauagcaaaugcugacugaacaugaaggucuuaauuagcucuaacugacu

SEQ ID NO: 13 - RNA, 57 nt, Homo sapiens
gaugaccccagguaacucugagugugucgcugaugccaucaccgcagcgcucugacc

SEQ ID NO: 14 - RNA, 22 nt, Homo sapiens
ugagguaguagguuguauaguu

SEQ ID NO: 15 - RNA, 22 nt, Homo sapiens
ugagguaguagguugugugguu

SEQ ID NO: 16 - RNA, 22 nt, Homo sapiens
ugagguaguagguuguaugguu

SEQ ID NO: 17 - RNA, 21 nt, Homo sapiens
agagguaguagguugcauagu

SEQ ID NO: 18 - RNA, 21 nt, Homo sapiens
ugagguaggagguuguauagu

SEQ ID NO: 19 - RNA, 22 nt, Homo sapiens
ugagguaguagauuguauaguu

SEQ ID NO: 20 - RNA, 21 nt, Homo sapiens
ugagguaguaguuuguacagu

SEQ ID NO: 21 - RNA, 21 nt, Homo sapiens
ugagguaguaguuugugcugu

SEQ ID NO: 22 - RNA, 21 nt, Homo sapiens
uggaauguaaagaaguaugua

SEQ ID NO: 23 - RNA, 22 nt, Homo sapiens
uggaagacuagugauuuuguug

SEQ ID NO: 24 - RNA, 23 nt, Homo sapiens
uacccuguagauccgaauuugug

SEQ ID NO: 25 - RNA, 22 nt, Homo sapiens
uacccuguagaaccgaauuugu

SEQ ID NO: 26 - RNA, 22 nt, Homo sapiens
uagcagcacauaaugguuugug

SEQ ID NO: 27 - RNA, 22 nt, Homo sapiens
uagcagcacaucaugguuuaca

SEQ ID NO: 28 - RNA, 22 nt, Homo sapiens
uagcagcacguaaauauuggcg

SEQ ID NO: 29 - RNA, 20 nt, Homo sapiens
acugcagugaaggcacuugu

SEQ ID NO: 30 - RNA, 24 nt, Homo sapiens
caaagugcuuacagugcagguagu

SEQ ID NO: 31 - RNA, 22 nt, Homo sapiens
uaaggugcaucuagugcagaua

SEQ ID NO: 32 - RNA, 22 nt, Homo sapiens
uagcuuaucagacugauguuga

SEQ ID NO: 33 - RNA, 22 nt, Homo sapiens
aagcugccaguugaagaacugu

SEQ ID NO: 34 - RNA, 21 nt, Homo sapiens
aucacauugccagggauuucc

SEQ ID NO: 35 - RNA, 21 nt, Homo sapiens
aucacauugccagggauuacc

SEQ ID NO: 36 - RNA, 22 nt, Homo sapiens
cauugcacuugucucggucuga

SEQ ID NO: 37 - RNA, 21 nt, Homo sapiens
uucaaguaauccaggauaggc

SEQ ID NO: 38 - RNA, 22 nt, Homo sapiens
uucaaguaauccaggauaggcu

SEQ ID NO: 39 - RNA, 22 nt, Homo sapiens
uucaaguaauucaggauagguu

SEQ ID NO: 40 - RNA, 21 nt, Homo sapiens
uucaaguaauucaggauaggu

SEQ ID NO: 41 - RNA, 21 nt, Homo sapiens
uucacaguggcuaaguuccgc

SEQ ID NO: 42 - RNA, 21 nt, Homo sapiens
uucacaguggcuaaguucugc

SEQ ID NO: 43 - RNA, 22 nt, Homo sapiens
aaggagcucacagucuauugag

SEQ ID NO: 44 - RNA, 21 nt, Homo sapiens
uagcaccaucugaaaucgguu

SEQ ID NO: 45 - RNA, 23 nt, Homo sapiens
uagcaccauuugaaaucaguguu

SEQ ID NO: 46 - RNA, 20 nt, Homo sapiens
uagcaccauuugaaaucggu

SEQ ID NO: 47 - RNA, 22 nt, Homo sapiens
cuuucagucggauguuugcagc

SEQ ID NO: 48 - RNA, 22 nt, Homo sapiens
uguaaacauccucgacuggaag

SEQ ID NO: 49 - RNA, 23 nt, Homo sapiens
uguaaacauccuacacucucagc

SEQ ID NO: 50 - RNA, 22 nt, Homo sapiens
uguaaacauccccgacuggaag

SEQ ID NO: 51 - RNA, 20 nt, Homo sapiens
uguaaacauccuugacugga

SEQ ID NO: 52 - RNA, 22 nt, Homo sapiens
cuuucagucggauguuuacagc

SEQ ID NO: 53 - RNA, 21 nt, Homo sapiens
uauugcacauuacuaaguugc

SEQ ID NO: 54 - RNA, 19 nt, Homo sapiens
gugcauuguaguugcauug

SEQ ID NO: 55 - RNA, 23 nt, Homo sapiens
uggcagugucuuagcugguuguu

SEQ ID NO: 56 - RNA, 22 nt, Homo sapiens
uggcagugucuuagcugguugu

SEQ ID NO: 57 - RNA, 23 nt, Homo sapiens
uaggcagugucauuagcugauug

SEQ ID NO: 58 - RNA, 23 nt, Homo sapiens
aggcaguguaguuagcugauugc

SEQ ID NO: 59 - RNA, 21 nt, Homo sapiens
uauugcacuugucccggccug

SEQ ID NO: 60 - RNA, 22 nt, Homo sapiens
uauugcacuugucccggccugu

SEQ ID NO: 61 - RNA, 22 nt, Homo sapiens
aaagugcuguucgugcagguag

SEQ ID NO: 62 - RNA, 22 nt, Homo sapiens
uucaacggguauuuauugagca

SEQ ID NO: 63 - RNA, 22 nt, Homo sapiens
uuuggcacuagcacauuuuugc

SEQ ID NO: 64 - RNA, 22 nt, Homo sapiens
aacccguagauccgaucuugug

SEQ ID NO: 65 - RNA, 22 nt, Homo sapiens
cacccguagaaccgaccuugcg

SEQ ID NO: 66 - RNA, 22 nt, Homo sapiens
aacccguagauccgaacuugug

SEQ ID NO: 67 - RNA, 22 nt, Homo sapiens
uacaguacugugauaacugaag

SEQ ID NO: 68 - RNA, 23 nt, Homo sapiens
agcagcauuguacagggcuauga

SEQ ID NO: 69 - RNA, 20 nt, Homo sapiens
ucaaaugcucagacuccugu

SEQ ID NO: 70 - RNA, 21 nt, Homo sapiens
uaaagugcugacagugcagau

SEQ ID NO: 71 - RNA, 23 nt, Homo sapiens
agcagcauuguacagggcuauca

SEQ ID NO: 72 - RNA, 23 nt, Homo sapiens
uggagugugacaaugguguuugu

SEQ ID NO: 73 - RNA, 22 nt, Homo sapiens
uuaaggcacgcggugaaugcca

SEQ ID NO: 74 - RNA, 23 nt, Homo sapiens
ucccugagacccuuuaaccugug

SEQ ID NO: 75 - RNA, 22 nt, Homo sapiens
ucccugagacccuaacuuguga

SEQ ID NO: 76 - RNA, 21 nt, Homo sapiens
ucguaccgugaguaauaaugc

SEQ ID NO: 77 - RNA, 21 nt, Homo sapiens
cauuauuacuuuugguacgcg

SEQ ID NO: 78 - RNA, 22 nt, Homo sapiens
ucggauccgucugagcuuggcu

SEQ ID NO: 79 - RNA, 22 nt, Homo sapiens
ucacagugaaccggucucuuuu

SEQ ID NO: 80 - RNA, 22 nt, Homo sapiens
cagugcaauguuaaaagggcau

SEQ ID NO: 81 - RNA, 22 nt, Homo sapiens
cagugcaaugaugaaagggcau

SEQ ID NO: 82 - RNA, 22 nt, Homo sapiens
uaacagucuacagccauggucg

SEQ ID NO: 83 - RNA, 22 nt, Homo sapiens
uugguccccuucaaccagcugu

SEQ ID NO: 84 - RNA, 21 nt, Homo sapiens
ugugacugguugaccagaggg

SEQ ID NO: 85 - RNA, 23 nt, Homo sapiens
uauggcuuuuuauuccuauguga

SEQ ID NO: 86 - RNA, 22 nt, Homo sapiens
uauggcuuuucauuccuaugug

SEQ ID NO: 87 - RNA, 21 nt, Homo sapiens
agugguuuuacccuaugguag

SEQ ID NO: 88 - RNA, 22 nt, Homo sapiens
ugagaugaagcacuguagcuca

SEQ ID NO: 89 - RNA, 24 nt, Homo sapiens
guccaguuuucccaggaaucccuu

SEQ ID NO: 90 - RNA, 22 nt, Homo sapiens
ugagaacugaauuccauggguu

SEQ ID NO: 91 - RNA, 20 nt, Homo sapiens
guguguggaaaugcuucugc

SEQ ID NO: 92 - RNA, 22 nt, Homo sapiens
ucagugcacuacagaacuuugu

SEQ ID NO: 93 - RNA, 22 nt, Homo sapiens
ucagugcaucacagaacuuugu

SEQ ID NO: 94 - RNA, 22 nt, Homo sapiens
ucuggcuccgugucuucacucc

SEQ ID NO: 95 - RNA, 22 nt, Homo sapiens
ucucccaacccuuguaccagug

SEQ ID NO: 96 - RNA, 22 nt, Homo sapiens
ucagugcaugacagaacuuggg

SEQ ID NO: 97 - RNA, 21 nt, Homo sapiens
ucagugcaugacagaacuugg

SEQ ID NO: 98 - RNA, 20 nt, Homo sapiens
uugcauagucacaaaaguga

SEQ ID NO: 99 - RNA, 20 nt, Homo sapiens
uaaggcacgcggugaaugcc

SEQ ID NO: 100 - RNA, 22 nt, Homo sapiens
uagguuauccguguugccuucg

SEQ ID NO: 101 - RNA, 22 nt, Homo sapiens
aaucauacacgguugaccuauu

SEQ ID NO: 102 - RNA, 20 nt, Homo sapiens
cuuccucgucugucugcccc

SEQ ID NO: 103 - RNA, 23 nt, Homo sapiens
aacauucaacgcugucggugagu

SEQ ID NO: 104 - RNA, 22 nt, Homo sapiens
aacauucaaccugucggugagu

SEQ ID NO: 105 - RNA, 21 nt, Homo sapiens
ugguucuagacuugccaacua

SEQ ID NO: 106 - RNA, 23 nt, Homo sapiens
uauggcacugguagaauucacug

SEQ ID NO: 107 - RNA, 22 nt, Homo sapiens
uggacggagaacugauaagggu

SEQ ID NO: 108 - RNA, 23 nt, Homo sapiens
caaagaauucuccuuuugggcuu

SEQ ID NO: 109 - RNA, 21 nt, Homo sapiens
ucgugucuuguguugcagccg

SEQ ID NO: 110 - RNA, 23 nt, Homo sapiens
gugccuacugagcugauaucagu

SEQ ID NO: 111 - RNA, 22 nt, Homo sapiens
ugauauguuugauauauuaggu

SEQ ID NO: 112 - RNA, 22 nt, Homo sapiens
caacggaaucccaaaagcagcu

SEQ ID NO: 113 - RNA, 21 nt, Homo sapiens
cugaccuaugaauugacagcc

SEQ ID NO: 114 - RNA, 21 nt, Homo sapiens
aacuggccuacaaagucccag

SEQ ID NO: 115 - RNA, 22 nt, Homo sapiens
uguaacagcaacuccaugugga

SEQ ID NO: 116 - RNA, 21 nt, Homo sapiens
uagcagcacagaaauauuggc

SEQ ID NO: 117 - RNA, 21 nt, Homo sapiens
uagguaguuucauguuguugg

SEQ ID NO: 118 - RNA, 21 nt, Homo sapiens
uagguaguuuccuguuguugg

SEQ ID NO: 119 - RNA, 22 nt, Homo sapiens
uucaccaccuucuccacccagc

SEQ ID NO: 120 - RNA, 23 nt, Homo sapiens
cccaguguucagacuaccuguuc

SEQ ID NO: 121 - RNA, 22 nt, Homo sapiens
uacaguagucugcacauugguu

SEQ ID NO: 122 - RNA, 23 nt, Homo sapiens
cccaguguuuagacuaucuguuc

SEQ ID NO: 123 - RNA, 22 nt, Homo sapiens
uaacacugucugguaacgaugu

SEQ ID NO: 124 - RNA, 22 nt, Homo sapiens
uaauacugccggguaaugaugg

SEQ ID NO: 125 - RNA, 22 nt, Homo sapiens
gugaaauguuuaggaccacuag

SEQ ID NO: 126 - RNA, 22 nt, Homo sapiens
uucccuuugucauccuaugccu

SEQ ID NO: 127 - RNA, 22 nt, Homo sapiens
uccuucauuccaccggagucug

SEQ ID NO: 128 - RNA, 22 nt, Homo sapiens
uggaauguaaggaagugugugg

SEQ ID NO: 129 - RNA, 22 nt, Homo sapiens
auaagacgagcaaaaagcuugu

SEQ ID NO: 130 - RNA, 22 nt, Homo sapiens
cugugcgugugacagcggcuga

SEQ ID NO: 131 - RNA, 22 nt, Homo sapiens
uucccuuugucauccuucgccu

SEQ ID NO: 132 - RNA, 21 nt, Homo sapiens
uaacagucuccagucacggcc

SEQ ID NO: 133 - RNA, 22 nt, Homo sapiens
accaucgaccguugauuguacc

SEQ ID NO: 134 - RNA, 21 nt, Homo sapiens
acagcaggcacagacaggcag

SEQ ID NO: 135 - RNA, 21 nt, Homo sapiens
augaccuaugaauugacagac

SEQ ID NO: 136 - RNA, 21 nt, Homo sapiens
uaaucucagcuggcaacugug

SEQ ID NO: 137 - RNA, 24 nt, Homo sapiens
uacugcaucaggaacugauuggau

SEQ ID NO: 138 - RNA, 21 nt, Homo sapiens
uugugcuugaucuaaccaugu

SEQ ID NO: 139 - RNA, 21 nt, Homo sapiens
ugauuguccaaacgcaauucu

SEQ ID NO: 140 - RNA, 21 nt, Homo sapiens
ccacaccguaucugacacuuu

SEQ ID NO: 141 - RNA, 23 nt, Homo sapiens
agcuacauugucugcuggguuuc

SEQ ID NO: 142 - RNA, 24 nt, Homo sapiens
agcuacaucuggcuacugggucuc

SEQ ID NO: 143 - RNA, 21 nt, Homo sapiens
agggcccccccucaauccugu

SEQ ID NO: 144 - RNA, 23 nt, Homo sapiens
cagugcaauaguauugucaaagc

SEQ ID NO: 145 - RNA, 23 nt, Homo sapiens
uaagugcuuccauguuuugguga

SEQ ID NO: 146 - RNA, 22 nt, Homo sapiens
uaaacguggauguacuugcuuu

SEQ ID NO: 147 - RNA, 23 nt, Homo sapiens
uaagugcuuccauguuuuaguag

SEQ ID NO: 148 - RNA, 23 nt, Homo sapiens
acuuuaacauggaagugcuuucu

SEQ ID NO: 149 - RNA, 23 nt, Homo sapiens
uaagugcuuccauguuucagugg

SEQ ID NO: 150 - RNA, 22 nt, Homo sapiens
uuuaacauggggguaccugcug

SEQ ID NO: 151 - RNA, 23 nt, Homo sapiens
uaagugcuuccauguuugagugu

SEQ ID NO: 152 - RNA, 23 nt, Homo sapiens
aaaagcuggguugagagggcgaa

SEQ ID NO: 153 - RNA, 22 nt, Homo sapiens
gcacauuacacggucgaccucu

SEQ ID NO: 154 - RNA, 23 nt, Homo sapiens
cgcauccccuagggcauuggugu

SEQ ID NO: 155 - RNA, 23 nt, Homo sapiens
ccuaguagguguccaguaagugu

SEQ ID NO: 156 - RNA, 20 nt, Homo sapiens
ccucugggcccuuccuccag

SEQ ID NO: 157 - RNA, 22 nt, Homo sapiens
cuggcccucucugcccuuccgu

SEQ ID NO: 158 - RNA, 23 nt, Homo sapiens
gcaaagcacacggccugcagaga

SEQ ID NO: 159 - RNA, 21 nt, Homo sapiens
gccccugggccuauccuagaa

SEQ ID NO: 160 - RNA, 23 nt, Homo sapiens
ucaagagcaauaacgaaaaaugu

SEQ ID NO: 161 - RNA, 23 nt, Homo sapiens
uccagcuccuauaugaugccuuu

SEQ ID NO: 162 - RNA, 23 nt, Homo sapiens
uccagcaucagugauuuuguuga

SEQ ID NO: 163 - RNA, 21 nt, Homo sapiens
ucccuguccuccaggagcuca

SEQ ID NO: 164 - RNA, 23 nt, Homo sapiens
uccgucucaguuacuuuauagcc

SEQ ID NO: 165 - RNA, 24 nt, Homo sapiens
ucucacacagaaaucgcacccguc

SEQ ID NO: 166 - RNA, 21 nt, Homo sapiens
ugcugacuccuaguccagggc

SEQ ID NO: 167 - RNA, 23 nt, Homo sapiens
ugucugcccgcaugccugccucu

SEQ ID NO: 168 - RNA, 22 nt, Homo sapiens
uuaucagaaucuccagggguac

SEQ ID NO: 169 - RNA, 22 nt, Homo sapiens
aauugcacuuuagcaaugguga

SEQ ID NO: 170 - RNA, 22 nt, Homo sapiens
acauagaggaaauuccacguuu

SEQ ID NO: 171 - RNA, 21 nt, Homo sapiens
aauaauacaugguugaucuuu

SEQ ID NO: 172 - RNA, 21 nt, Homo sapiens
gccugcugggguggaaccugg

SEQ ID NO: 173 - RNA, 21 nt, Homo sapiens
gugccgccaucuuuugagugu

SEQ ID NO: 174 - RNA, 23 nt, Homo sapiens
aaagugcugcgacauuugagcgu

SEQ ID NO: 175 - RNA, 23 nt, Homo sapiens
gaagugcuucgauuuuggggugu

SEQ ID NO: 176 - RNA, 22 nt, Homo sapiens
acucaaaaugggggcgcuuucc

SEQ ID NO: 177 - RNA, 22 nt, Homo sapiens
uuauaauacaaccugauaagug

SEQ ID NO: 178 - RNA, 22 nt, Homo sapiens
uuuguucguucggcucgcguga

SEQ ID NO: 179 - RNA, 21 nt, Homo sapiens
aucauagaggaaaauccacgu

SEQ ID NO: 180 - RNA, 22 nt, Homo sapiens
aucacacaaaggcaacuuuugu

SEQ ID NO: 181 - RNA, 22 nt, Homo sapiens
cuccugacuccagguccugugu

SEQ ID NO: 182 - RNA, 19 nt, Homo sapiens
ugguagacuauggaacgua

SEQ ID NO: 183 - RNA, 22 nt, Homo sapiens
uauguaauaugguccacaucuu

SEQ ID NO: 184 - RNA, 22 nt, Homo sapiens
ugguugaccauagaacaugcgc

SEQ ID NO: 185 - RNA, 22 nt, Homo sapiens
uauacaagggcaagcucucugu

SEQ ID NO: 186 - RNA, 22 nt, Homo sapiens
gaaguuguucgugguggauucg

SEQ ID NO: 187 - RNA, 22 nt, Homo sapiens
agaucagaaggugauuguggcu

SEQ ID NO: 188 - RNA, 20 nt, Homo sapiens
auuccuagaaauuguucaua

SEQ ID NO: 189 - RNA, 22 nt, Homo sapiens
cuggacuuggagucagaaggcc

SEQ ID NO: 190 - RNA, 22 nt, Homo sapiens
agcucggucugaggccccucag

SEQ ID NO: 191 - RNA, 22 nt, Homo sapiens
ugagguaguaaguuguauuguu

SEQ ID NO: 192 - RNA, 24 nt, Homo sapiens
aaaagugcuuacagugcagguagc

SEQ ID NO: 193 - RNA, 22 nt, Homo sapiens
ccacugccccaggugcugcugg

SEQ ID NO: 194 - RNA, 23 nt, Homo sapiens
uaaagugcuuauagugcagguag

SEQ ID NO: 195 - RNA, 19 nt, Homo sapiens
gguccagaggggagauagg

SEQ ID NO: 196 - RNA, 23 nt, Homo sapiens
ucuuugguuaucuagcuguauga

SEQ ID NO: 197 - RNA, 21 nt, Homo sapiens
uaaagcuagauaaccgaaagu

SEQ ID NO: 198 - RNA, 22 nt, Homo sapiens
uagcaccauuugaaaucgguua

SEQ ID NO: 199 - RNA, 22 nt, Homo sapiens
ucacagugaaccggucucuuuc

SEQ ID NO: 200 - RNA, 21 nt, Homo sapiens
cuuuuugcggucugggcuugc

SEQ ID NO: 201 - RNA, 21 nt, Homo sapiens
uugguccccuucaaccagcua

SEQ ID NO: 202 - RNA, 23 nt, Homo sapiens
acuccauuuguuuugaugaugga

SEQ ID NO: 203 - RNA, 22 nt, Homo sapiens
uauugcuuaagaauacgcguag

SEQ ID NO: 204 - RNA, 17 nt, Homo sapiens
agcugguguugugaauc

SEQ ID NO: 205 - RNA, 22 nt, Homo sapiens
acuagacugaagcuccuugagg

SEQ ID NO: 206 - RNA, 22 nt, Homo sapiens
uuuggcaaugguagaacucaca

SEQ ID NO: 207 - RNA, 18 nt, Homo sapiens
uggagagaaaggcaguuc

SEQ ID NO: 208 - RNA, 23 nt, Homo sapiens
caagucacuagugguuccguuua

SEQ ID NO: 209 - RNA, 22 nt, Homo sapiens
ugguuuaccgucccacauacau

SEQ ID NO: 210 - RNA, 22 nt, Homo sapiens
uguaaacauccuacacucagcu

SEQ ID NO: 211 - RNA, 22 nt, Homo sapiens
cuggacuuagggucagaaggcc

SEQ ID NO: 212 - RNA, 22 nt, Homo sapiens
cagcagcaauucauguuuugaa

SEQ ID NO: 213 - RNA, 55 nt, Homo sapiens
auuugcuaucugagagauggugaugacauuuuaaaccaccaagaucgcugaugca

SEQ ID NO: 214 - RNA, 70 nt, Homo sapiens
guaacuguggugauggaaauguguuagccucagacacuacugaggugguucuuucuauccuaguacaguc

SEQ ID NO: 215 - RNA, 61 nt, Homo sapiens
uugcaccucugagaguggaaugacuccuguggaguugauccuagucugggugcaaacaauu

SEQ ID NO: 216 - RNA, 48 nt, Homo sapiens
ccaguucugcuacugacaguaagugaagauaaagugugucugaggaga

SEQ ID NO: 217 - RNA, 57 nt, Homo sapiens
cacuaauaggaagugccgucagaagcgauaacugacgaagacuacuccugucugauu

SEQ ID NO: 218 - RNA, 22 nt, Homo sapiens
caucccuugcaugguggagggu

SEQ ID NO: 219 - RNA, 22 nt, Homo sapiens
uaaggugcaucuagugcaguua

SEQ ID NO: 220 - RNA, 24 nt, Homo sapiens
aacuggcccucaaagucccgcuuu

SEQ ID NO: 221 - RNA, 22 nt, Homo sapiens
caucuuaccggacagugcugga

SEQ ID NO: 222 - RNA, 22 nt, Homo sapiens
agagguauagggcaugggaaaa

SEQ ID NO: 223 - RNA, 22 nt, Homo sapiens
uuuccuaugcauauacuucuuu

SEQ ID NO: 224 - RNA, 23 nt, Homo sapiens
caaagugcucauagugcagguag

SEQ ID NO: 225 - RNA, 22 nt, Homo sapiens
uaugugggaugguaaaccgcuu

SEQ ID NO: 226 - RNA, 22 nt, Homo sapiens
uaaugccccuaaaaauccuuau

SEQ ID NO: 227 - RNA, 22 nt, Homo sapiens
agaucgaccguguuauauucgc

SEQ ID NO: 228 - RNA, 22 nt, Homo sapiens
agguuacccgagcaacuuugca

SEQ ID NO: 229 - RNA, 23 nt, Homo sapiens
acuucaccugguccacuagccgu

SEQ ID NO: 230 - RNA, 22 nt, Homo sapiens
uaauacugucugguaaaaccgu

SEQ ID NO: 231 - RNA, 23 nt, Homo sapiens
ucuuggaguaggucauugggugg

SEQ ID NO: 232 - RNA, 21 nt, Homo sapiens
cuggauggcuccuccaugucu

SEQ ID NO: 233 - RNA, 22 nt, Homo sapiens
aucaugaugggcuccucggugu

SEQ ID NO: 234 - RNA, 22 nt, Homo sapiens
uugcauauguaggaugucccau

SEQ ID NO: 235 - RNA, 22 nt, Homo sapiens
uggcaguguauuguuagcuggu

SEQ ID NO: 236 - RNA, 22 nt, Homo sapiens
uuuuugcgauguguuccuaaua

SEQ ID NO: 237 - RNA, 22 nt, Homo sapiens
uguuugcagaggaaacugagac

SEQ ID NO: 238 - RNA, 21 nt, Homo sapiens
ucagucucaucugcaaagaag

SEQ ID NO: 239 - RNA, 22 nt, Homo sapiens
gagguuguccguggugaguucg

SEQ ID NO: 240 - RNA, 22 nt, Homo sapiens
agaggcuggccgugaugaauuc

SEQ ID NO: 241 - RNA, 22 nt, Homo sapiens
caaccuggaggacuccaugcug

SEQ ID NO: 242 - RNA, 23 nt, Homo sapiens
aguggggaacccuuccaugagga

SEQ ID NO: 243 - RNA, 21 nt, Homo sapiens
gccccugggccuauccuagaa

SEQ ID NO: 244 - RNA, 21 nt, Homo sapiens
agggcccccccucaauccugu

SEQ ID NO: 245 - RNA, 23 nt, Homo sapiens
aggaccugcgggacaagauucuu

SEQ ID NO: 246 - RNA, 22 nt, Homo sapiens
uuguacaugguaggcuuucauu

SEQ ID NO: 247 - RNA, 24 nt, Homo sapiens
ugaaacauacacgggaaaccucuu

SEQ ID NO: 248 - RNA, 17 nt, Homo sapiens
auuacauggccaaucuc

SEQ ID NO: 249 - RNA, 21 nt, Homo sapiens
cagcagcacacugugguuugu

SEQ ID NO: 250 - RNA, 23 nt, Homo sapiens
uuucaagccagggggcguuuuuc

SEQ ID NO: 251 - RNA, 23 nt, Homo sapiens
uuaagacuugcagugauguuuaa

SEQ ID NO: 252 - RNA, 22 nt, Homo sapiens
augcaccugggcaaggauucug

SEQ ID NO: 253 - RNA, 22 nt, Homo sapiens
aauccuuugucccugggugaga

SEQ ID NO: 254 - RNA, 23 nt, Homo sapiens
uagcagcgggaacaguucugcag

SEQ ID NO: 255 - RNA, 22 nt, Homo sapiens
gucaacacuugcugguuuccuc

SEQ ID NO: 256 - RNA, 21 nt, Homo sapiens
uaaggcacccuucugaguaga

SEQ ID NO: 257 - RNA, 21 nt, Homo sapiens
uuuugcaccuuuuggagugaa

SEQ ID NO: 258 - RNA, 23 nt, Homo sapiens
ugauuguagccuuuuggaguaga

SEQ ID NO: 259 - RNA, 23 nt, Homo sapiens
ugauugguacgucuguggguaga

SEQ ID NO: 260 - RNA, 67 nt, Homo sapiens
ugguauugccauugcuucacuguuggcuuugaccaggguaugaucucuuaaucuucucucugagcug

SEQ ID NO: 261 - RNA, 42 nt, Homo sapiens
cgcaaggaugacacgcaaauucgugaagcguuccauauuuuu

SEQ ID NO: 262 - RNA, 23 nt, Homo sapiens
gacacgggcgacagcugcggccc

SEQ ID NO: 263 - RNA, 52 nt, Homo sapiens
gaacuuauugacgggcggacagaaacugugugcugauugucacguucugauu

SEQ ID NO: 264 - RNA, 18 nt, Homo sapiens
ucuacagugcacgugucu

SEQ ID NO: 265 - RNA, 22 nt, Homo sapiens
aacauucauugcugucgguggg

SEQ ID NO: 266 - RNA, 24 nt, Homo sapiens
aacauucauuguugucgguggguu

SEQ ID NO: 267 - RNA, 21 nt, Homo sapiens
ggcaagaugcuggcauagcug

SEQ ID NO: 268 - RNA, 22 nt, Homo sapiens
aacacaccugguuaaccucuuu

SEQ ID NO: 269 - RNA, 22 nt, Homo sapiens
aucauagaggaaaauccauguu

SEQ ID NO: 270 - RNA, 21 nt, Homo sapiens
aucgggaaugucguguccgcc

SEQ ID NO: 271 - RNA, 23 nt, Homo sapiens
aaaccguuaccauuacugaguuu

SEQ ID NO: 272 - RNA, 21 nt, Homo sapiens
cccagauaauggcacucucaa

SEQ ID NO: 273 - RNA, 23 nt, Homo sapiens
agugacaucacauauacggcagc

SEQ ID NO: 274 - RNA, 23 nt, Homo sapiens
aaacaaacauggugcacuucuuu

SEQ ID NO: 275 - RNA, 21 nt, Homo sapiens
auccuugcuaucugggugcua

SEQ ID NO: 276 - RNA, 21 nt, Homo sapiens
agacccuggucugcacucuau

SEQ ID NO: 277 - RNA, 21 nt, Homo sapiens
gugucuuuugcucugcaguca

SEQ ID NO: 278 - RNA, 24 nt, Homo sapiens
uucuccaaaagaaagcacuuucug

SEQ ID NO: 279 - RNA, 22 nt, Homo sapiens
ccucuagauggaagcacugucu

SEQ ID NO: 280 - RNA, 23 nt, Homo sapiens
aaagugcauccuuuuagagguuu

SEQ ID NO: 281 - RNA, 21 nt, Homo sapiens
aaagugcuuccuuuuagaggg

SEQ ID NO: 282 - RNA, 23 nt, Homo sapiens
aaagugcuuccuuuuagaggguu

SEQ ID NO: 283 - RNA, 23 nt, Homo sapiens
aaagugcuucucuuugguggguu

SEQ ID NO: 284 - RNA, 21 nt, Homo sapiens
aaagugcuuccuuuuugaggg

SEQ ID NO: 285 - RNA, 22 nt, Homo sapiens
aagugcuuccuuuuagaggguu

SEQ ID NO: 286 - RNA, 24 nt, Homo sapiens
acaaagugcuucccuuuagagugu

SEQ ID NO: 287 - RNA, 22 nt, Homo sapiens
aacgcacuucccuuuagagugu

SEQ ID NO: 288 - RNA, 21 nt, Homo sapiens
gaaggcgcuucccuuuagagc

SEQ ID NO: 289 - RNA, 21 nt, Homo sapiens
cucuagagggaagcacuuucu

SEQ ID NO: 290 - RNA, 21 nt, Homo sapiens
aaagugcuuccuuuuagaggc

SEQ ID NO: 291 - RNA, 23 nt, Homo sapiens
uacucaggagaguggcaaucaca

SEQ ID NO: 292 - RNA, 23 nt, Homo sapiens
cacucagccuugagggcacuuuc

SEQ ID NO: 293 - RNA, 22 nt, Homo sapiens
uucacagggaggugucauuuau

SEQ ID NO: 294 - RNA, 20 nt, Homo sapiens
auugacacuucugugaguag

SEQ ID NO: 295 - RNA, 21 nt, Homo sapiens
gagugccuucuuuuggagcgu

SEQ ID NO: 296 - RNA, 18 nt, Homo sapiens
ugcuuccuuucagagggu

SEQ ID NO: 297 - RNA, 22 nt, Homo sapiens
aucuggagguaagaagcacuuu

SEQ ID NO: 298 - RNA, 23 nt, Homo sapiens
aucgugcaucccuuuagaguguu

SEQ ID NO: 299 - RNA, 22 nt, Homo sapiens
ucgugcaucccuuuagaguguu

SEQ ID NO: 300 - RNA, 22 nt, Homo sapiens
aucgugcauccuuuuagagugu

SEQ ID NO: 301 - RNA, 21 nt, Homo sapiens
aaagcgcuucccuuugcugga

SEQ ID NO: 302 - RNA, 22 nt, Homo sapiens
caaagcgcuccccuuuagaggu

SEQ ID NO: 303 - RNA, 22 nt, Homo sapiens
caaagcgcuucucuuuagagug

SEQ ID NO: 304 - RNA, 23 nt, Homo sapiens
ucucuggagggaagcacuuucug

SEQ ID NO: 305 - RNA, 21 nt, Homo sapiens
caaagcgcuucccuuuggagc

SEQ ID NO: 306 - RNA, 22 nt, Homo sapiens
aaagcgcuucccuucagagugu

SEQ ID NO: 307 - RNA, 21 nt, Homo sapiens
aaagcgcuucucuuuagagga

SEQ ID NO: 308 - RNA, 25 nt, Homo sapiens
aaagugcauccuuuuagaguguuac

SEQ ID NO: 309 - RNA, 22 nt, Homo sapiens
aaagugcaucuuuuuagaggau

SEQ ID NO: 310 - RNA, 23 nt, Homo sapiens
caaagugccucccuuuagagugu

SEQ ID NO: 311 - RNA, 22 nt, Homo sapiens
aaagugccuccuuuuagagugu

SEQ ID NO: 312 - RNA, 22 nt, Homo sapiens
uucuccaaaagggagcacuuuc

SEQ ID NO: 313 - RNA, 22 nt, Homo sapiens
aaagugcuucccuuuggacugu

SEQ ID NO: 314 - RNA, 21 nt, Homo sapiens
cuccagagggaaguacuuucu

SEQ ID NO: 315 - RNA, 23 nt, Homo sapiens
ucuacaaagggaagcccuuucug

SEQ ID NO: 316 - RNA, 22 nt, Homo sapiens
acaaagugcuucccuuuagagu

SEQ ID NO: 317 - RNA, 23 nt, Homo sapiens
aaaaugguucccuuuagaguguu

SEQ ID NO: 318 - RNA, 21 nt, Homo sapiens
aacgcgcuucccuauagaggg

SEQ ID NO: 319 - RNA, 21 nt, Homo sapiens
gaaggcgcuucccuuuggagu

SEQ ID NO: 320 - RNA, 21 nt, Homo sapiens
cuccagagggaugcacuuucu

SEQ ID NO: 321 - RNA, 24 nt, Homo sapiens
cucuugagggaagcacuuucuguu

SEQ ID NO: 322 - RNA, 24 nt, Homo sapiens
cucuagagggaagcgcuuucuguu

SEQ ID NO: 323 - RNA, 21 nt, Homo sapiens
cugcaaagggaagcccuuucu

SEQ ID NO: 324 - RNA, 70 nt, Homo sapiens
caguagugaugaaauuccacuucauugguccguguuucugaaccacaugauuuucucggauguucugaug

SEQ ID NO: 325 - RNA, 66 nt, Homo sapiens
cugcgaugauggcauuucuuaggacaccuuuggauuaauaaugaaaacaacuacucucugagcagc

SEQ ID NO: 326 - RNA, 65 nt, Homo sapiens
cugcagugaugacuuucuugggacaccuuuggauuuaccgugaaaauuaauaaauucugagcagc

SEQ ID NO: 327 - RNA, 70 nt, Homo sapiens
cuuaaugaugacuguuuuuuuugauugcuugaagcaaugugaaaaacacauuucaccggcucugaaagcu

SEQ ID NO: 328 - RNA, 63 nt, Homo sapiens
uggcgaugaggagguaccuauuguguugaguaacggugauaauuuuauacgcuauucugagcc

SEQ ID NO: 329 - RNA, 57 nt, Homo sapiens
ccagucacagauuucuuuguuccuucuccacucccacugcaucacuuaacuagccuu

SEQ ID NO: 330 - RNA, 60 nt, Homo sapiens
agccugugaugcuuuaagaguaguggacagaagggauuucugaaauucuauucugaggcu

SEQ ID NO: 331 - RNA, 65 nt, Homo sapiens
uaaugauucugccaaaugaaauauaaugauaucacuguaaaaccguuccauuuugauucugaggu

SEQ ID NO: 332 - RNA, 22 nt, Homo sapiens
aauugcacgguauccaucugua

SEQ ID NO: 333 - RNA, 21 nt, Homo sapiens
acugcccuaagugcuccuucu

SEQ ID NO: 334 - RNA, 24 nt, Homo sapiens
aauccuuggaaccuaggugugagu

SEQ ID NO: 335 - RNA, 21 nt, Homo sapiens
aauauaacacagauggccugu

SEQ ID NO: 336 - RNA, 23 nt, Homo sapiens
ucacuccucuccucccgucuucu

SEQ ID NO: 337 - RNA, 22 nt, Homo sapiens
gucauacacggcucuccucucu

SEQ ID NO: 338 - RNA, 22 nt, Homo sapiens
uccuguacugagcugccccgag

SEQ ID NO: 339 - RNA, 22 nt, Homo sapiens
aaucauacagggacauccaguu

SEQ ID NO: 340 - RNA, 22 nt, Homo sapiens
uaugugccuuuggacuacaucg

SEQ ID NO: 341 - RNA, 23 nt, Homo sapiens
caucuggagguaagaagcacuuu

SEQ ID NO: 342 - RNA, 21 nt, Homo sapiens
ugaaggucuacugugugccag

SEQ ID NO: 343 - RNA, 22 nt, Homo sapiens
cggguggaucacgaugcaauuu

SEQ ID NO: 344 - RNA, 22 nt, Homo sapiens
ugugacagauugauaacugaaa

SEQ ID NO: 345 - RNA, 22 nt, Homo sapiens
aaucguacagggucauccacuu

SEQ ID NO: 346 - RNA, 22 nt, Homo sapiens
ggagaaauuauccuuggugugu

SEQ ID NO: 347 - RNA, 21 nt, Homo sapiens
gguagauucuccuucuaugag

SEQ ID NO: 348 - RNA, 22 nt, Homo sapiens
ucggggaucaucaugucacgag

SEQ ID NO: 349 - RNA, 22 nt, Homo sapiens
aucagcaaacauuuauugugug

SEQ ID NO: 350 - RNA, 20 nt, Homo sapiens
auucugcauuuuuagcaagu

SEQ ID NO: 351 - RNA, 21 nt, Homo sapiens
aauauuauacagucaaccucu

SEQ ID NO: 352 - RNA, 21 nt, Homo sapiens
ugacaacuauggaugagcucu

SEQ ID NO: 353 - RNA, 23 nt, Homo sapiens
ggcagguucucacccucucuagg

SEQ ID NO: 354 - RNA, 25 nt, Homo sapiens
ggcggagggaaguagguccguuggu

SEQ ID NO: 355 - RNA, 22 nt, Homo sapiens
cuugguucagggagggucccca

SEQ ID NO: 356 - RNA, 22 nt, Homo sapiens
uacccauugcauaucggaguug

SEQ ID NO: 357 - RNA, 23 nt, Homo sapiens
aaugacacgaucacucccguuga

SEQ ID NO: 358 - RNA, 23 nt, Homo sapiens
aauggcgccacuaggguugugca

SEQ ID NO: 359 - RNA, 22 nt, Homo sapiens
caugccuugaguguaggaccgu

SEQ ID NO: 360 - RNA, 21 nt, Homo sapiens
gcgacccacucuugguuucca

SEQ ID NO: 361 - RNA, 21 nt, Homo sapiens
aacaggugacugguuagacaa

SEQ ID NO: 362 - RNA, 21 nt, Homo sapiens
aaaacggugagauuuuguuuu

SEQ ID NO: 363 - RNA, 21 nt, Homo sapiens
gcuaguccugacucagccagu

SEQ ID NO: 364 - RNA, 21 nt, Homo sapiens
aggguaagcugaaccucugau

SEQ ID NO: 365 - RNA, 20 nt, Homo sapiens
gaugagcucauuguaauaug

SEQ ID NO: 366 - RNA, 23 nt, Homo sapiens
guuugcacgggugggccuugucu

SEQ ID NO: 367 - RNA, 19 nt, Homo sapiens
ugagcugcuguaccaaaau

SEQ ID NO: 368 - RNA, 21 nt, Homo sapiens
uaaaguaaauaugcaccaaaa

SEQ ID NO: 369 - RNA, 22 nt, Homo sapiens
caaaguuuaagauccuugaagu

SEQ ID NO: 370 - RNA, 20 nt, Homo sapiens
aaaguagcuguaccauuugc

SEQ ID NO: 371 - RNA, 19 nt, Homo sapiens
agguugacauacguuuccc

SEQ ID NO: 372 - RNA, 19 nt, Homo sapiens
aggcacggugucagcaggc

SEQ ID NO: 373 - RNA, 22 nt, Homo sapiens
ggcuggcucgcgaugucuguuu

SEQ ID NO: 374 - RNA, 19 nt, Homo sapiens
gggcgccugugaucccaac

SEQ ID NO: 375 - RNA, 23 nt, Homo sapiens
aguauguucuuccaggacagaac

SEQ ID NO: 376 - RNA, 21 nt, Homo sapiens
gcgacccauacuugguuucag

SEQ ID NO: 377 - RNA, 21 nt, Homo sapiens
aguuaaugaauccuggaaagu

SEQ ID NO: 378 - RNA, 22 nt, Homo sapiens
gaaaacagcaauuaccuuugca

SEQ ID NO: 379 - RNA, 22 nt, Homo sapiens
caaaacuggcaauuacuuuugc

SEQ ID NO: 380 - RNA, 22 nt, Homo sapiens
uaugcauuguauuuuuaggucc

SEQ ID NO: 381 - RNA, 21 nt, Homo sapiens
uuuccauaggugaugagucac

SEQ ID NO: 382 - RNA, 22 nt, Homo sapiens
caagaaccucaguugcuuuugu

SEQ ID NO: 383 - RNA, 21 nt, Homo sapiens
uuggccacaauggguuagaac

SEQ ID NO: 384 - RNA, 24 nt, Homo sapiens
ucagaacaaaugccgguucccaga

SEQ ID NO: 385 - RNA, 22 nt, Homo sapiens
ugucuuacucccucaggcacau

SEQ ID NO: 386 - RNA, 20 nt, Homo sapiens
agaccauggguucucauugu

SEQ ID NO: 387 - RNA, 22 nt, Homo sapiens
uugugucaauaugcgaugaugu

SEQ ID NO: 388 - RNA, 25 nt, Homo sapiens
aggcaccagccaggcauugcucagc

SEQ ID NO: 389 - RNA, 25 nt, Homo sapiens
cccaucugggguggccugugacuuu

SEQ ID NO: 390 - RNA, 21 nt, Homo sapiens
aagccugcccggcuccucggg

SEQ ID NO: 391 - RNA, 22 nt, Homo sapiens
ugugucacucgaugaccacugu

SEQ ID NO: 392 - RNA, 21 nt, Homo sapiens
acagucugcugagguuggagc

SEQ ID NO: 393 - RNA, 20 nt, Homo sapiens
guugugucaguuuaucaaac

SEQ ID NO: 394 - RNA, 23 nt, Homo sapiens
aucccuugcaggggcuguugggu

SEQ ID NO: 395 - RNA, 23 nt, Homo sapiens
acuuacagacaagagccuugcuc

SEQ ID NO: 396 - RNA, 22 nt, Homo sapiens
uaguaccaguaccuuguguuca

SEQ ID NO: 397 - RNA, 22 nt, Homo sapiens
uggucuaggauuguuggaggag

SEQ ID NO: 398 - RNA, 19 nt, Homo sapiens
agcugucugaaaaugucuu

SEQ ID NO: 399 - RNA, 22 nt, Homo sapiens
gugagucucuaagaaaagagga

SEQ ID NO: 400 - RNA, 20 nt, Homo sapiens
ucuaguaagaguggcagucg

SEQ ID NO: 401 - RNA, 22 nt, Homo sapiens
guucucccaacguaagcccagc

SEQ ID NO: 402 - RNA, 22 nt, Homo sapiens
aguauucuguaccagggaaggu

SEQ ID NO: 403 - RNA, 21 nt, Homo sapiens
agaccuggcccagaccucagc

SEQ ID NO: 404 - RNA, 21 nt, Homo sapiens
gugcauugcuguugcauugca

SEQ ID NO: 405 - RNA, 22 nt, Homo sapiens
cacacacugcaauuacuuuugc

SEQ ID NO: 406 - RNA, 19 nt, Homo sapiens
aggcugcggaauucaggac

SEQ ID NO: 407 - RNA, 23 nt, Homo sapiens
uaaaucccauggugccuucuccu

SEQ ID NO: 408 - RNA, 21 nt, Homo sapiens
aaacuacugaaaaucaaagau

SEQ ID NO: 409 - RNA, 21 nt, Homo sapiens
guucaaauccagaucuauaac

SEQ ID NO: 410 - RNA, 25 nt, Homo sapiens
aggggugguguugggacagcuccgu

SEQ ID NO: 411 - RNA, 19 nt, Homo sapiens
gugucugcuuccuguggga

SEQ ID NO: 412 - RNA, 20 nt, Homo sapiens
aggguguuucucucaucucu

SEQ ID NO: 413 - RNA, 23 nt, Homo sapiens
cuaauaguaucuaccacaauaaa

SEQ ID NO: 414 - RNA, 21 nt, Homo sapiens
ugagcuaaaugugugcuggga

SEQ ID NO: 415 - RNA, 22 nt, Homo sapiens
aaccagcaccccaacuuuggac

SEQ ID NO: 416 - RNA, 23 nt, Homo sapiens
acuugggcacugaaacaaugucc

SEQ ID NO: 417 - RNA, 25 nt, Homo sapiens
gcugggcagggcuucugagcuccuu

SEQ ID NO: 418 - RNA, 24 nt, Homo sapiens
ugugcuugcucgucccgcccgcag

SEQ ID NO: 419 - RNA, 24 nt, Homo sapiens
acugggggcuuucgggcucugcgu

SEQ ID NO: 420 - RNA, 25 nt, Homo sapiens
agggaucgcgggcggguggcggccu

SEQ ID NO: 421 - RNA, 23 nt, Homo sapiens
aucgcugcgguugcgagcgcugu

SEQ ID NO: 422 - RNA, 21 nt, Homo sapiens
augauccaggaaccugccucu

SEQ ID NO: 423 - RNA, 24 nt, Homo sapiens
aaagacauaggauagagucaccuc

SEQ ID NO: 424 - RNA, 20 nt, Homo sapiens
aggaauguuccuucuuugcc

SEQ ID NO: 425 - RNA, 23 nt, Homo sapiens
gaacgccuguucuugccaggugg

SEQ ID NO: 426 - RNA, 21 nt, Homo sapiens
uccgagccugggucucccucu

SEQ ID NO: 427 - RNA, 22 nt, Homo sapiens
acucaaaacccuucagugacuu

SEQ ID NO: 428 - RNA, 22 nt, Homo sapiens
caaaaaucucaauuacuuuugc

SEQ ID NO: 429 - RNA, 22 nt, Homo sapiens
agacuucccauuugaagguggc

SEQ ID NO: 430 - RNA, 22 nt, Homo sapiens
gucccucuccaaaugugucuug

SEQ ID NO: 431 - RNA, 23 nt, Homo sapiens
aaacucuacuuguccuucugagu

SEQ ID NO: 432 - RNA, 22 nt, Homo sapiens
acuuguaugcuagcucagguag

SEQ ID NO: 433 - RNA, 24 nt, Homo sapiens
gaccuggacauguuugugcccagu

SEQ ID NO: 434 - RNA, 19 nt, Homo sapiens
aguguggcuuucuuagagc

SEQ ID NO: 435 - RNA, 19 nt, Homo sapiens
ucuaggcugguacugcuga

SEQ ID NO: 436 - RNA, 21 nt, Homo sapiens
ggcuagcaacagcgcuuaccu

SEQ ID NO: 437 - RNA, 19 nt, Homo sapiens
aagcagcugccucugaggc

SEQ ID NO: 438 - RNA, 21 nt, Homo sapiens
guggcugcacucacuuccuuc

SEQ ID NO: 439 - RNA, 19 nt, Homo sapiens
aagugugcagggcacuggu

SEQ ID NO: 440 - RNA, 22 nt, Homo sapiens
aaaccuguguuguucaagaguc

SEQ ID NO: 441 - RNA, 21 nt, Homo sapiens
aggaggcagcgcucucaggac

SEQ ID NO: 442 - RNA, 22 nt, Homo sapiens
uuuaggauaagcuugacuuuug

SEQ ID NO: 443 - RNA, 22 nt, Homo sapiens
caaaaaccacaguuucuuuugc

SEQ ID NO: 444 - RNA, 24 nt, Homo sapiens
ugccugggucucuggccugcgcgu

SEQ ID NO: 445 - RNA, 21 nt, Homo sapiens
ucccacguuguggcccagcag

SEQ ID NO: 446 - RNA, 22 nt, Homo sapiens
aggcaguguauuguuagcuggc

SEQ ID NO: 447 - RNA, 21 nt, Homo sapiens
uugaaacaaucucuacugaac

SEQ ID NO: 448 - RNA, 21 nt, Homo sapiens
uaguagaccguauagcguacg

SEQ ID NO: 449 - RNA, 22 nt, Homo sapiens
uggugggccgcagaacaugugc

SEQ ID NO: 450 - RNA, 22 nt, Homo sapiens
auaauacaugguuaaccucuuu

SEQ ID NO: 451 - RNA, 21 nt, Homo sapiens
ugaguuggccaucugagugag

SEQ ID NO: 452 - RNA, 20 nt, Homo sapiens
guccgcucggcgguggccca

SEQ ID NO: 453 - RNA, 24 nt, Homo sapiens
cugaagugauguguaacugaucag

SEQ ID NO: 454 - RNA, 19 nt, Homo sapiens
gagccaguuggacaggagc

SEQ ID NO: 455 - RNA, 23 nt, Homo sapiens
auucuaauuucuccacgucuuug

SEQ ID NO: 456 - RNA, 21 nt, Homo sapiens
cuucuugugcucuaggauugu

SEQ ID NO: 457 - RNA, 23 nt, Homo sapiens
auucauuugguauaaaccgcgau

SEQ ID NO: 458 - RNA, 22 nt, Homo sapiens
uugagaaugaugaaucauuagg

SEQ ID NO: 459 - RNA, 21 nt, Homo sapiens
ucuuguguucucuagaucagu

SEQ ID NO: 460 - RNA, 21 nt, Homo sapiens
caaagaggaaggucccauuac

SEQ ID NO: 461 - RNA, 22 nt, Homo sapiens
uuaugguuugccugggacugag

SEQ ID NO: 462 - RNA, 19 nt, Homo sapiens
ugggcguaucuguaugcua

SEQ ID NO: 463 - RNA, 65 nt, Homo sapiens
uggcagugaugaucacaaauccguguuucugacaagcgauugacgauagaaaaccggcugagcca

SEQ ID NO: 464 - RNA, 23 nt, Homo sapiens
uaauacugccugguaaugaugac

SEQ ID NO: 465 - RNA, 22 nt, Homo sapiens
ucaggcucaguccccucccgau

SEQ ID NO: 466 - RNA, 22 nt, Homo sapiens
aagugcugucauagcugagguc

SEQ ID NO: 467 - RNA, 21 nt, Homo sapiens
ugucuugcaggccgucaugca

SEQ ID NO: 468 - RNA, 22 nt, Homo sapiens
cuacaaagggaagcacuuucuc

SEQ ID NO: 469 - RNA, 23 nt, Homo sapiens
uuacaguuguucaaccaguuacu

SEQ ID NO: 470 - RNA, 22 nt, Homo sapiens
gagcuuauucauaaaagugcag

SEQ ID NO: 471 - RNA, 22 nt, Homo sapiens
acuccagccccacagccucagc

SEQ ID NO: 472 - RNA, 21 nt, Homo sapiens
gaagugugccguggugugucu

SEQ ID NO: 473 - RNA, 22 nt, Homo sapiens
uacgucaucguugucaucguca

SEQ ID NO: 474 - RNA, 22 nt, Homo sapiens
uuugugaccugguccacuaacc

SEQ ID NO: 475 - RNA, 23 nt, Homo sapiens
ugucacucggcucggcccacuac

SEQ ID NO: 476 - RNA, 23 nt, Homo sapiens
ugcaccaugguugucugagcaug

SEQ ID NO: 477 - RNA, 24 nt, Homo sapiens
gauugcucugcgugcggaaucgac

SEQ ID NO: 478 - RNA, 23 nt, Homo sapiens
ucugcucauaccccaugguuucu

SEQ ID NO: 479 - RNA, 22 nt, Homo sapiens
acccuaucaauauugucucugc

SEQ ID NO: 480 - RNA, 22 nt, Homo sapiens
ugagaccucuggguucugagcu

SEQ ID NO: 481 - RNA, 26 nt, Homo sapiens
guuggaggaugaaaguacggagugau

SEQ ID NO: 482 - RNA, 28 nt, Homo sapiens
ucacaaugcugacacucaaacugcugac

SEQ ID NO: 483 - RNA, 23 nt, Homo sapiens
uccaguaccacgugucagggcca

SEQ ID NO: 484 - RNA, 23 nt, Homo sapiens
cugggaucuccggggucuugguu

SEQ ID NO: 485 - RNA, 23 nt, Homo sapiens
caguaacaaagauucauccuugu

SEQ ID NO: 486 - RNA, 23 nt, Homo sapiens
uggugcggagagggcccacagug

SEQ ID NO: 487 - RNA, 22 nt, Homo sapiens
gcacugagaugggaguggugua

SEQ ID NO: 488 - RNA, 22 nt, Homo sapiens
aaugcaccugggcaaggauuca

SEQ ID NO: 489 - RNA, 22 nt, Homo sapiens
agacccuggucugcacucuauc

SEQ ID NO: 490 - RNA, 20 nt, Homo sapiens
gugcauugcuguugcauugc

SEQ ID NO: 491 - RNA, 22 nt, Homo sapiens
gggagccaggaaguauugaugu

SEQ ID NO: 492 - RNA, 23 nt, Homo sapiens
ugugcuugcucgucccgcccgca

SEQ ID NO: 493 - RNA, 22 nt, Homo sapiens
cgucaacacuugcugguuuccu

SEQ ID NO: 494 - RNA, 18 nt, Homo sapiens
uucacagggaggugucau

SEQ ID NO: 495 - RNA, 23 nt, Homo sapiens
uaaauuucaccuuucugagaagg

SEQ ID NO: 496 - RNA, 23 nt, Homo sapiens
uacuccagagggcgucacucaug

SEQ ID NO: 497 - RNA, 21 nt, Homo sapiens
cggggcagcucaguacaggau

SEQ ID NO: 498 - RNA, 22 nt, Homo sapiens
augguuccgucaagcaccaugg

SEQ ID NO: 499 - RNA, 22 nt, Homo sapiens
agaguugagucuggacgucccg

SEQ ID NO: 500 - RNA, 22 nt, Homo sapiens
accuggcauacaauguagauuu

SEQ ID NO: 501 - RNA, 22 nt, Homo sapiens
cucaguagccaguguagauccu

SEQ ID NO: 502 - RNA, 22 nt, Homo sapiens
cguguauuugacaagcugaguu

SEQ ID NO: 503 - RNA, 21 nt, Homo sapiens
caagucacuagugguuccguu

SEQ ID NO: 504 - RNA, 22 nt, Homo sapiens
caucaucgucucaaaugagucu

SEQ ID NO: 505 - RNA, 22 nt, Homo sapiens
gaggguuggguggaggcucucc

SEQ ID NO: 506 - RNA, 22 nt, Homo sapiens
caaucacuaacuccacugccau

SEQ ID NO: 507 - RNA, 22 nt, Homo sapiens
aggggcuggcuuuccucugguc

SEQ ID NO: 508 - RNA, 22 nt, Homo sapiens
gcccaaaggugaauuuuuuggg

SEQ ID NO: 509 - RNA, 21 nt, Homo sapiens
cucccacaugcaggguuugca

SEQ ID NO: 510 - RNA, 22 nt, Homo sapiens
ccaauauuggcugugcugcucc

SEQ ID NO: 511 - RNA, 22 nt, Homo sapiens
cugggagaggguuguuuacucc

SEQ ID NO: 512 - RNA, 22 nt, Homo sapiens
uauugcacauuacuaaguugca

SEQ ID NO: 513 - RNA, 22 nt, Homo sapiens
cugggagaaggcuguuuacucu

SEQ ID NO: 514 - RNA, 22 nt, Homo sapiens
caauuuagugugugugauauuu

SEQ ID NO: 515 - RNA, 22 nt, Homo sapiens
uagcaccaucugaaaucgguua

SEQ ID NO: 516 - RNA, 22 nt, Homo sapiens
ugcuaugccaacauauugccau

SEQ ID NO: 517 - RNA, 21 nt, Homo sapiens
acucuuucccuguugcacuac

SEQ ID NO: 518 - RNA, 22 nt, Homo sapiens
ccuauucuugauuacuuguuuc

SEQ ID NO: 519 - RNA, 23 nt, Homo sapiens
ucccccaggugugauucugauuu

SEQ ID NO: 520 - RNA, 22 nt, Homo sapiens
aacacaccuauucaaggauuca

SEQ ID NO: 521 - RNA, 21 nt, Homo sapiens
cuguacaggccacugccuugc

SEQ ID NO: 522 - RNA, 22 nt, Homo sapiens
acuuuaacauggaagugcuuuc

SEQ ID NO: 523 - RNA, 22 nt, Homo sapiens
acuuuaacauggaggcacuugc

SEQ ID NO: 524 - RNA, 22 nt, Homo sapiens
acuguugcuaauaugcaacucu

SEQ ID NO: 525 - RNA, 21 nt, Homo sapiens
aacauagaggaaauuccacgu

SEQ ID NO: 526 - RNA, 23 nt, Homo sapiens
aagugccgccaucuuuugagugu

SEQ ID NO: 527 - RNA, 22 nt, Homo sapiens
cuuaucagauuguauuguaauu

SEQ ID NO: 528 - RNA, 22 nt, Homo sapiens
uggguuccuggcaugcugauuu

SEQ ID NO: 529 - RNA, 22 nt, Homo sapiens
guagauucuccuucuaugagua

SEQ ID NO: 530 - RNA, 22 nt, Homo sapiens
agagguugcccuuggugaauuc

SEQ ID NO: 531 - RNA, 22 nt, Homo sapiens
cugggagguggauguuuacuuc

SEQ ID NO: 532 - RNA, 22 nt, Homo sapiens
aacgccauuaucacacuaaaua

SEQ ID NO: 533 - RNA, 22 nt, Homo sapiens
uucacauugugcuacugucugc

SEQ ID NO: 534 - RNA, 22 nt, Homo sapiens
accguggcuuucgauuguuacu

SEQ ID NO: 535 - RNA, 22 nt, Homo sapiens
uauguaacaugguccacuaacu

SEQ ID NO: 536 - RNA, 22 nt, Homo sapiens
aaaguucugagacacuccgacu

SEQ ID NO: 537 - RNA, 21 nt, Homo sapiens
gugcauuguaguugcauugca

SEQ ID NO: 538 - RNA, 22 nt, Homo sapiens
caauguuuccacagugcaucac

SEQ ID NO: 539 - RNA, 23 nt, Homo sapiens
agguugggaucgguugcaaugcu

SEQ ID NO: 540 - RNA, 22 nt, Homo sapiens
ggguggggauuuguugcauuac

SEQ ID NO: 541 - RNA, 22 nt, Homo sapiens
acugcugagcuagcacuucccg

SEQ ID NO: 542 - RNA, 22 nt, Homo sapiens
aaucaugugcagugccaauaug

SEQ ID NO: 543 - RNA, 22 nt, Homo sapiens
caagcucgcuucuaugggucug

SEQ ID NO: 544 - RNA, 22 nt, Homo sapiens
caagcuuguaucuauagguaug

SEQ ID NO: 545 - RNA, 22 nt, Homo sapiens
caguuaucacagugcugaugcu

SEQ ID NO: 546 - RNA, 22 nt, Homo sapiens
gcuauuucacgacaccaggguu

SEQ ID NO: 547 - RNA, 22 nt, Homo sapiens
caucuuccaguacaguguugga

SEQ ID NO: 548 - RNA, 22 nt, Homo sapiens
ggugcagugcugcaucucuggu

SEQ ID NO: 549 - RNA, 21 nt, Homo sapiens
aggggugcuaucugugauuga

SEQ ID NO: 550 - RNA, 22 nt, Homo sapiens
ggauaucaucauauacuguaag

SEQ ID NO: 551 - RNA, 22 nt, Homo sapiens
ggauuccuggaaauacuguucu

SEQ ID NO: 552 - RNA, 20 nt, Homo sapiens
ggggagcuguggaagcagua

SEQ ID NO: 553 - RNA, 25 nt, Homo sapiens
cuagugagggacagaaccaggauuc

SEQ ID NO: 554 - RNA, 23 nt, Homo sapiens
gcagcagagaauaggacuacguc

SEQ ID NO: 555 - RNA, 21 nt, Homo sapiens
gucagcggaggaaaagaaacu

SEQ ID NO: 556 - RNA, 20 nt, Homo sapiens
agagucuugugaugucuugc

SEQ ID NO: 557 - RNA, 22 nt, Homo sapiens
uacugcagacguggcaaucaug

SEQ ID NO: 558 - RNA, 21 nt, Homo sapiens
gaacggcuucauacaggaguu

SEQ ID NO: 559 - RNA, 22 nt, Homo sapiens
cuccuauaugaugccuuucuuc

SEQ ID NO: 560 - RNA, 22 nt, Homo sapiens
ucacaagucaggcucuugggac

SEQ ID NO: 561 - RNA, 22 nt, Homo sapiens
auguagggcuaaaagccauggg

SEQ ID NO: 562 - RNA, 22 nt, Homo sapiens
aaguucuguuauacacucaggc

SEQ ID NO: 563 - RNA, 20 nt, Homo sapiens
acugccccaggugcugcugg

SEQ ID NO: 564 - RNA, 22 nt, Homo sapiens
gcuacuucacaacaccagggcc

SEQ ID NO: 565 - RNA, 22 nt, Homo sapiens
ccucugaaauucaguucuucag

SEQ ID NO: 566 - RNA, 21 nt, Homo sapiens
agggagggacgggggcugugc

SEQ ID NO: 567 - RNA, 24 nt, Homo sapiens
gcugguuucauauggugguuuaga

SEQ ID NO: 568 - RNA, 22 nt, Homo sapiens
cugguuucacaugguggcuuag

SEQ ID NO: 569 - RNA, 23 nt, Homo sapiens
ucaaaugcucagacuccuguggu

SEQ ID NO: 570 - RNA, 22 nt, Homo sapiens
acggauguuugagcaugugcua

SEQ ID NO: 571 - RNA, 23 nt, Homo sapiens
aaaagugcuuacagugcagguag

SEQ ID NO: 572 - RNA, 22 nt, Homo sapiens
cugcaauguaagcacuucuuac

SEQ ID NO: 573 - RNA, 22 nt, Homo sapiens
ccaauauuacugugcugcuuua

SEQ ID NO: 574 - RNA, 22 nt, Homo sapiens
cugcgcaagcuacugccuugcu

SEQ ID NO: 575 - RNA, 22 nt, Homo sapiens
cgaaucauuauuugcugcucua

SEQ ID NO: 576 - RNA, 22 nt, Homo sapiens
agagcuuagcugauuggugaac

SEQ ID NO: 577 - RNA, 22 nt, Homo sapiens
ugugcgcagggagaccucuccc

SEQ ID NO: 578 - RNA, 22 nt, Homo sapiens
ugucuacuacuggagacacugg

SEQ ID NO: 579 - RNA, 23 nt, Homo sapiens
ccaguuaccgcuuccgcuaccgc

SEQ ID NO: 580 - RNA, 22 nt, Homo sapiens
acaguagagggaggaaucgcag

SEQ ID NO: 581 - RNA, 22 nt, Homo sapiens
auccgcgcucugacucucugcc

SEQ ID NO: 582 - RNA, 22 nt, Homo sapiens
ugcccuuaaaggugaacccagu

SEQ ID NO: 583 - RNA, 24 nt, Homo sapiens
uggggagcugaggcucugggggug

SEQ ID NO: 584 - RNA, 23 nt, Homo sapiens
cacccggcugugugcacaugugc

SEQ ID NO: 585 - RNA, 23 nt, Homo sapiens
ugagcgccucgacgacagagccg

SEQ ID NO: 586 - RNA, 22 nt, Homo sapiens
uuuuucauuauugcuccugacc

SEQ ID NO: 587 - RNA, 22 nt, Homo sapiens
gcugacuccuaguccagggcuc

SEQ ID NO: 588 - RNA, 22 nt, Homo sapiens
ucuucucuguuuuggccaugug

SEQ ID NO: 589 - RNA, 21 nt, Homo sapiens
cugacuguugccguccuccag

SEQ ID NO: 590 - RNA, 22 nt, Homo sapiens
aaauuauuguacaucggaugag

SEQ ID NO: 591 - RNA, 24 nt, Homo sapiens
agcagaagcagggagguucuccca

SEQ ID NO: 592 - RNA, 22 nt, Homo sapiens
ugcaacgaaccugagccacuga

SEQ ID NO: 593 - RNA, 23 nt, Homo sapiens
cgggucggaguuagcucaagcgg

SEQ ID NO: 594 - RNA, 21 nt, Homo sapiens
cgcgggugcuuacugacccuu

SEQ ID NO: 595 - RNA, 21 nt, Homo sapiens
cacuguguccuuucugcguag

SEQ ID NO: 596 - RNA, 22 nt, Homo sapiens
caagcucgugucuguggguccg

SEQ ID NO: 597 - RNA, 22 nt, Homo sapiens
cguguucacagcggaccuugau

SEQ ID NO: 598 - RNA, 24 nt, Homo sapiens
ucccugagacccuuuaaccuguga

SEQ ID NO: 599 - RNA, 22 nt, Homo sapiens
acaggugagguucuugggagcc

SEQ ID NO: 600 - RNA, 25 nt, Homo sapiens
aaaggauucugcugucggucccacu

SEQ ID NO: 601 - RNA, 22 nt, Homo sapiens
uggugggcacagaaucuggacu

SEQ ID NO: 602 - RNA, 21 nt, Homo sapiens
uuaauaucggacaaccauugu

SEQ ID NO: 603 - RNA, 22 nt, Homo sapiens
uauaccucaguuuuaucaggug

SEQ ID NO: 604 - RNA, 21 nt, Homo sapiens
ccuggaaacacugagguugug

SEQ ID NO: 605 - RNA, 22 nt, Homo sapiens
uggauuucuuugugaaucacca

SEQ ID NO: 606 - RNA, 21 nt, Homo sapiens
ccaccaccgugucugacacuu

SEQ ID NO: 607 - RNA, 22 nt, Homo sapiens
uuuugcaauauguuccugaaua

SEQ ID NO: 608 - RNA, 22 nt, Homo sapiens
uugggaucauuuugcauccaua

SEQ ID NO: 609 - RNA, 21 nt, Homo sapiens
uacuuggaaaggcaucaguug

SEQ ID NO: 610 - RNA, 22 nt, Homo sapiens
ugcaacuuaccugagucauuga

SEQ ID NO: 611 - RNA, 22 nt, Homo sapiens
acacagggcuguugugaagacu

SEQ ID NO: 612 - RNA, 21 nt, Homo sapiens
uacucaaaaagcugucaguca

SEQ ID NO: 613 - RNA, 22 nt, Homo sapiens
gacugacaccucuuugggugaa

SEQ ID NO: 614 - RNA, 22 nt, Homo sapiens
cacuggcuccuuucuggguaga

SEQ ID NO: 615 - RNA, 22 nt, Homo sapiens
uagguaguuuccuguuguuggg

SEQ ID NO: 616 - RNA, 21 nt, Homo sapiens
ucacagugaaccggucucuuu

SEQ ID NO: 617 - RNA, 23 nt, Homo sapiens
uaaggugcaucuagugcaguuag

SEQ ID NO: 618 - RNA, 23 nt, Homo sapiens
uacccuguagaaccgaauuugug

SEQ ID NO: 619 - RNA, 22 nt, Homo sapiens
uaaucucagcuggcaacuguga

SEQ ID NO: 620 - RNA, 22 nt, Homo sapiens
ugagguaguaguuugugcuguu

SEQ ID NO: 621 - RNA, 22 nt, Homo sapiens
uggaauguaaagaaguauguau

SEQ ID NO: 622 - RNA, 22 nt, Homo sapiens
uguaaacauccuugacuggaag

SEQ ID NO: 623 - RNA, 22 nt, Homo sapiens
uggugguuuacaaaguaauuca

SEQ ID NO: 624 - RNA, 21 nt, Homo sapiens
cacauuacacggucgaccucu

SEQ ID NO: 625 - RNA, 22 nt, Homo sapiens
ucguaccgugaguaauaaugcg

SEQ ID NO: 626 - RNA, 22 nt, Homo sapiens
cugaagcucagagggcucugau

SEQ ID NO: 627 - RNA, 22 nt, Homo sapiens
ucucugggccugugucuuaggc

SEQ ID NO: 628 - RNA, 22 nt, Homo sapiens
auaaagcuagauaaccgaaagu

SEQ ID NO: 629 - RNA, 22 nt, Homo sapiens
uauagggauuggagccguggcg

SEQ ID NO: 630 - RNA, 22 nt, Homo sapiens
cuagguauggucccagggaucc

SEQ ID NO: 631 - RNA, 21 nt, Homo sapiens
uaccacaggguagaaccacgg

SEQ ID NO: 632 - RNA, 21 nt, Homo sapiens
uacugcagacaguggcaauca

SEQ ID NO: 633 - RNA, 22 nt, Homo sapiens
ugauugguacgucuguggguag

SEQ ID NO: 634 - RNA, 22 nt, Homo sapiens
aaaaguaauugugguuuuugcc

SEQ ID NO: 635 - RNA, 22 nt, Homo sapiens
uauguaacacgguccacuaacc

SEQ ID NO: 636 - RNA, 22 nt, Homo sapiens
uaugucugcugaccaucaccuu

SEQ ID NO: 637 - RNA, 23 nt, Homo sapiens
ucggggaucaucaugucacgaga

SEQ ID NO: 638 - RNA, 22 nt, Homo sapiens
uacucaggagaguggcaaucac

SEQ ID NO: 639 - RNA, 21 nt, Homo sapiens
acuggacuuggagucagaagg

SEQ ID NO: 640 - RNA, 21 nt, Homo sapiens
gcaguccaugggcauauacac

SEQ ID NO: 641 - RNA, 22 nt, Homo sapiens
uggagugugacaaugguguuug

SEQ ID NO: 642 - RNA, 22 nt, Homo sapiens
uuugguccccuucaaccagcug

SEQ ID NO: 643 - RNA, 22 nt, Homo sapiens
uuugguccccuucaaccagcua

SEQ ID NO: 644 - RNA, 22 nt, Homo sapiens
aauccuuugucccugggugaga

SEQ ID NO: 645 - RNA, 21 nt, Homo sapiens
ugagaugaagcacuguagcuc

SEQ ID NO: 646 - RNA, 22 nt, Homo sapiens
aacuggccuacaaagucccagu

SEQ ID NO: 647 - RNA, 22 nt, Homo sapiens
uaauacugccugguaaugauga

SEQ ID NO: 648 - RNA, 22 nt, Homo sapiens
uccagcaucagugauuuuguug

SEQ ID NO: 649 - RNA, 21 nt, Homo sapiens
uacaguacugugauaacugaa

SEQ ID NO: 650 - RNA, 21 nt, Homo sapiens
cuagacugaagcuccuugagg

SEQ ID NO: 651 - RNA, 23 nt, Homo sapiens
ucuggcuccgugucuucacuccc

SEQ ID NO: 652 - RNA, 23 nt, Homo sapiens
ucccuguccuccaggagcucacg

SEQ ID NO: 653 - RNA, 22 nt, Homo sapiens
uuauaaagcaaugagacugauu

SEQ ID NO: 654 - RNA, 22 nt, Homo sapiens
uccgucucaguuacuuuauagc

SEQ ID NO: 655 - RNA, 23 nt, Homo sapiens
ucucacacagaaaucgcacccgu

SEQ ID NO: 656 - RNA, 23 nt, Homo sapiens
uauggcuuuucauuccuauguga

SEQ ID NO: 657 - RNA, 22 nt, Homo sapiens
gugugcggaaaugcuucugcua

SEQ ID NO: 658 - RNA, 21 nt, Homo sapiens
ugauauguuugauauuggguu

SEQ ID NO: 659 - RNA, 21 nt, Homo sapiens
aagguuacuuguuaguucagg

SEQ ID NO: 660 - RNA, 22 nt, Homo sapiens
auucugcauuuuuagcaaguuc

SEQ ID NO: 661 - RNA, 22 nt, Homo sapiens
ucaguaaauguuuauuagauga

SEQ ID NO: 662 - RNA, 22 nt, Homo sapiens
ucagcaaacauuuauugugugc

SEQ ID NO: 663 - RNA, 22 nt, Homo sapiens
cugcccuggcccgagggaccga

SEQ ID NO: 664 - RNA, 22 nt, Homo sapiens
uauggcacugguagaauucacu

SEQ ID NO: 665 - RNA, 22 nt, Homo sapiens
gugaauuaccgaagggccauaa

SEQ ID NO: 666 - RNA, 22 nt, Homo sapiens
uggagagaaaggcaguuccuga

SEQ ID NO: 667 - RNA, 22 nt, Homo sapiens
cugccaauuccauaggucacag

SEQ ID NO: 668 - RNA, 22 nt, Homo sapiens
gguccagaggggagauagguuc

SEQ ID NO: 669 - RNA, 22 nt, Homo sapiens
caucuuacugggcagcauugga

SEQ ID NO: 670 - RNA, 22 nt, Homo sapiens
gccugcugggguggaaccuggu

SEQ ID NO: 671 - RNA, 21 nt, Homo sapiens
agcuacaucuggcuacugggu

SEQ ID NO: 672 - RNA, 22 nt, Homo sapiens
aaaagcuggguugagagggcga

SEQ ID NO: 673 - RNA, 23 nt, Homo sapiens
guccaguuuucccaggaaucccu

SEQ ID NO: 674 - RNA, 21 nt, Homo sapiens
aggcaagaugcuggcauagcu

SEQ ID NO: 675 - RNA, 22 nt, Homo sapiens
ugggucuuugcgggcgagauga

SEQ ID NO: 676 - RNA, 22 nt, Homo sapiens
ugagguaguaguuuguacaguu

SEQ ID NO: 677 - RNA, 22 nt, Homo sapiens
agagguaguagguugcauaguu

SEQ ID NO: 678 - RNA, 23 nt, Homo sapiens
agcugguguugugaaucaggccg

SEQ ID NO: 679 - RNA, 22 nt, Homo sapiens
caaagaauucuccuuuugggcu

SEQ ID NO: 680 - RNA, 22 nt, Homo sapiens
cgucuuacccagcaguguuugg

SEQ ID NO: 681 - RNA, 22 nt, Homo sapiens
cuccuacauauuagcauuaaca

SEQ ID NO: 682 - RNA, 22 nt, Homo sapiens
caaauucguaucuaggggaaua

SEQ ID NO: 683 - RNA, 22 nt, Homo sapiens
ucuacagugcacgugucuccag

SEQ ID NO: 684 - RNA, 22 nt, Homo sapiens
auaagacgaacaaaagguuugu

SEQ ID NO: 685 - RNA, 21 nt, Homo sapiens
guguugaaacaaucucuacug

SEQ ID NO: 686 - RNA, 22 nt, Homo sapiens
ugccugucuacacuugcugugc

SEQ ID NO: 687 - RNA, 22 nt, Homo sapiens
caugguucugucaagcaccgcg

SEQ ID NO: 688 - RNA, 22 nt, Homo sapiens
aaucacuaaccacacggccagg

SEQ ID NO: 689 - RNA, 22 nt, Homo sapiens
uccauuacacuacccugccucu

SEQ ID NO: 690 - RNA, 22 nt, Homo sapiens
acuggacuuagggucagaaggc

SEQ ID NO: 691 - RNA, 22 nt, Homo sapiens
aagcccuuaccccaaaaaguau

SEQ ID NO: 692 - RNA, 23 nt, Homo sapiens
caacggaaucccaaaagcagcug

SEQ ID NO: 693 - RNA, 23 nt, Homo sapiens
uaauacugccggguaaugaugga

SEQ ID NO: 694 - RNA, 22 nt, Homo sapiens
aguucuucaguggcaagcuuua

SEQ ID NO: 695 - RNA, 22 nt, Homo sapiens
aucgggaaugucguguccgccc

SEQ ID NO: 696 - RNA, 22 nt, Homo sapiens
uuuugcgauguguuccuaauau

SEQ ID NO: 697 - RNA, 22 nt, Homo sapiens
acaguagucugcacauugguua

SEQ ID NO: 698 - RNA, 22 nt, Homo sapiens
cuuucagucagauguuugcugc

SEQ ID NO: 699 - RNA, 22 nt, Homo sapiens
acagcaggcacagacaggcagu

SEQ ID NO: 700 - RNA, 21 nt, Homo sapiens
cuauacaaucuacugucuuuc

SEQ ID NO: 701 - RNA, 21 nt, Homo sapiens
caaaacgugaggcgcugcuau

SEQ ID NO: 702 - RNA, 22 nt, Homo sapiens
ugcccuaaaugccccuucuggc

SEQ ID NO: 703 - RNA, 22 nt, Homo sapiens
acuguaguaugggcacuuccag

SEQ ID NO: 704 - RNA, 22 nt, Homo sapiens
caggucgucuugcagggcuucu

SEQ ID NO: 705 - RNA, 22 nt, Homo sapiens
ggagacgcggcccuguuggagu

SEQ ID NO: 706 - RNA, 22 nt, Homo sapiens
caacaaaucccagucuaccuaa

SEQ ID NO: 707 - RNA, 22 nt, Homo sapiens
acagauucgauucuaggggaau

SEQ ID NO: 708 - RNA, 22 nt, Homo sapiens
caaucagcaaguauacugcccu

SEQ ID NO: 709 - RNA, 22 nt, Homo sapiens
accacugaccguugacuguacc

SEQ ID NO: 710 - RNA, 23 nt, Homo sapiens
agguuguccguggugaguucgca

SEQ ID NO: 711 - RNA, 21 nt, Homo sapiens
caucccuugcaugguggaggg

SEQ ID NO: 712 - RNA, 21 nt, Homo sapiens
uccgguucucagggcuccacc

SEQ ID NO: 713 - RNA, 23 nt, Homo sapiens
uagugcaauauugcuuauagggu

SEQ ID NO: 714 - RNA, 22 nt, Homo sapiens
ugcggggcuagggcuaacagca

SEQ ID NO: 715 - RNA, 22 nt, Homo sapiens
cuguugccacuaaccucaaccu

SEQ ID NO: 716 - RNA, 22 nt, Homo sapiens
aaaucucugcaggcaaauguga

SEQ ID NO: 717 - RNA, 23 nt, Homo sapiens
ugagguugguguacuguguguga

SEQ ID NO: 718 - RNA, 20 nt, Homo sapiens
cggcucugggucugugggga

SEQ ID NO: 719 - RNA, 22 nt, Homo sapiens
aacuguuugcagaggaaacuga

SEQ ID NO: 720 - RNA, 22 nt, Homo sapiens
cucaucugcaaagaaguaagug

SEQ ID NO: 721 - RNA, 23 nt, Homo sapiens
agguuacccgagcaacuuugcau

SEQ ID NO: 722 - RNA, 22 nt, Homo sapiens
gaauguugcucggugaaccccu

SEQ ID NO: 723 - RNA, 22 nt, Homo sapiens
aaccaucgaccguugaguggac

SEQ ID NO: 724 - RNA, 24 nt, Homo sapiens
uuuggcaaugguagaacucacacu

SEQ ID NO: 725 - RNA, 22 nt, Homo sapiens
cggcaacaagaaacugccugag

SEQ ID NO: 726 - RNA, 23 nt, Homo sapiens
uacugcaucaggaacugauugga

SEQ ID NO: 727 - RNA, 22 nt, Homo sapiens
aagacgggaggaaagaagggag

SEQ ID NO: 728 - RNA, 21 nt, Homo sapiens
ucacuccucuccucccgucuu

SEQ ID NO: 729 - RNA, 23 nt, Homo sapiens
ugaggggcagagagcgagacuuu

SEQ ID NO: 730 - RNA, 23 nt, Homo sapiens
aaggagcuuacaaucuagcuggg

SEQ ID NO: 731 - RNA, 22 nt, Homo sapiens
caacuagacugugagcuucuag

SEQ ID NO: 732 - RNA, 22 nt, Homo sapiens
agggacgggacgcggugcagug

SEQ ID NO: 733 - RNA, 22 nt, Homo sapiens
gaugagcucauuguaauaugag

SEQ ID NO: 734 - RNA, 22 nt, Homo sapiens
auauuaccauuagcucaucuuu

SEQ ID NO: 735 - RNA, 22 nt, Homo sapiens
gaaaucaagcgugggugagacc

SEQ ID NO: 736 - RNA, 22 nt, Homo sapiens
cgaaaacagcaauuaccuuugc

SEQ ID NO: 737 - RNA, 22 nt, Homo sapiens
cacgcucaugcacacacccaca

SEQ ID NO: 738 - RNA, 22 nt, Homo sapiens
auucuaauuucuccacgucuuu

SEQ ID NO: 739 - RNA, 22 nt, Homo sapiens
aagauguggaaaaauuggaauc

SEQ ID NO: 740 - RNA, 21 nt, Homo sapiens
aauggcgccacuaggguugug

SEQ ID NO: 741 - RNA, 22 nt, Homo sapiens
ggggguccccggugcucggauc

SEQ ID NO: 742 - RNA, 23 nt, Homo sapiens
uauucagauuagugccagucaug

SEQ ID NO: 743 - RNA, 22 nt, Homo sapiens
ccucccacacccaaggcuugca

SEQ ID NO: 744 - RNA, 21 nt, Homo sapiens
gcaggaacuugugagucuccu

SEQ ID NO: 745 - RNA, 21 nt, Homo sapiens
uugaaaggcuauuucuugguc

SEQ ID NO: 746 - RNA, 22 nt, Homo sapiens
gugacaucacauauacggcagc

SEQ ID NO: 747 - RNA, 22 nt, Homo sapiens
cuuaugcaagauucccuucuac

SEQ ID NO: 748 - RNA, 22 nt, Homo sapiens
ugcccuguggacucaguucugg

SEQ ID NO: 749 - RNA, 22 nt, Homo sapiens
uuccuaugcauauacuucuuug

SEQ ID NO: 750 - RNA, 20 nt, Homo sapiens
agagguauagggcaugggaa

SEQ ID NO: 751 - RNA, 22 nt, Homo sapiens
ugaaggucuacugugugccagg

SEQ ID NO: 752 - RNA, 22 nt, Homo sapiens
ugaaacauacacgggaaaccuc

SEQ ID NO: 753 - RNA, 22 nt, Homo sapiens
cgggguuuugagggcgagauga

SEQ ID NO: 754 - RNA, 22 nt, Homo sapiens
aacuggcccucaaagucccgcu

SEQ ID NO: 755 - RNA, 22 nt, Homo sapiens
caaaccacacugugguguuaga

SEQ ID NO: 756 - RNA, 22 nt, Homo sapiens
gagugccuucuuuuggagcguu

SEQ ID NO: 757 - RNA, 22 nt, Homo sapiens
aagugccuccuuuuagaguguu

SEQ ID NO: 758 - RNA, 22 nt, Homo sapiens
cucuagagggaagcgcuuucug

SEQ ID NO: 759 - RNA, 22 nt, Homo sapiens
aggcagcgggguguaguggaua

SEQ ID NO: 760 - RNA, 22 nt, Homo sapiens
gugaacgggcgccaucccgagg

SEQ ID NO: 761 - RNA, 22 nt, Homo sapiens
aaacauucgcggugcacuucuu

SEQ ID NO: 762 - RNA, 22 nt, Homo sapiens
acggguuaggcucuugggagcu

SEQ ID NO: 763 - RNA, 22 nt, Homo sapiens
ccaguggggcugcuguuaucug

SEQ ID NO: 764 - RNA, 22 nt, Homo sapiens
ccgcacuguggguacuugcugc

SEQ ID NO: 765 - RNA, 23 nt, Homo sapiens
acuuaaacguggauguacuugcu

SEQ ID NO: 766 - RNA, 23 nt, Homo sapiens
cucuugagggaagcacuuucugu

SEQ ID NO: 767 - RNA, 22 nt, Homo sapiens
gaaagugcuuccuuuuagaggc

SEQ ID NO: 768 - RNA, 22 nt, Homo sapiens
aaagugcauccuuuuagagguu

SEQ ID NO: 769 - RNA, 22 nt, Homo sapiens
gaaggcgcuucccuuuagagcg

SEQ ID NO: 770 - RNA, 23 nt, Homo sapiens
gaacgcgcuucccuauagagggu

SEQ ID NO: 771 - RNA, 22 nt, Homo sapiens
cucuagagggaagcacuuucuc

SEQ ID NO: 772 - RNA, 21 nt, Homo sapiens
gaaagcgcuucucuuuagagg

SEQ ID NO: 773 - RNA, 22 nt, Homo sapiens
cucuagagggaagcacuuucug

SEQ ID NO: 774 - RNA, 22 nt, Homo sapiens
agaauuguggcuggacaucugu

SEQ ID NO: 775 - RNA, 22 nt, Homo sapiens
cuuagcagguuguauuaucauu

SEQ ID NO: 776 - RNA, 23 nt, Homo sapiens
cagugcaaugauauugucaaagc

SEQ ID NO: 777 - RNA, 20 nt, Homo sapiens
cuacaaagggaagcccuuuc

SEQ ID NO: 778 - RNA, 21 nt, Homo sapiens
aaagcgcuucccuucagagug

SEQ ID NO: 779 - RNA, 20 nt, Homo sapiens
cugcaaagggaagcccuuuc

SEQ ID NO: 780 - RNA, 22 nt, Homo sapiens
gaaagcgcuucccuuugcugga

SEQ ID NO: 781 - RNA, 23 nt, Homo sapiens
uucauuugguauaaaccgcgauu

SEQ ID NO: 782 - RNA, 22 nt, Homo sapiens
uaacugguugaacaacugaacc

SEQ ID NO: 783 - RNA, 22 nt, Homo sapiens
aaagugcuuccuuuuagagggu

SEQ ID NO: 784 - RNA, 23 nt, Homo sapiens
caaagcgcuucucuuuagagugu

SEQ ID NO: 785 - RNA, 22 nt, Homo sapiens
aucgugcaucccuuuagagugu

SEQ ID NO: 786 - RNA, 22 nt, Homo sapiens
caaagugccucccuuuagagug

SEQ ID NO: 787 - RNA, 22 nt, Homo sapiens
cuauacaaccuacugccuuccc

SEQ ID NO: 788 - RNA, 22 nt, Homo sapiens
uagaguuacacccugggaguua

SEQ ID NO: 789 - RNA, 22 nt, Homo sapiens
ugagguaggagguuguauaguu

SEQ ID NO: 790 - RNA, 22 nt, Homo sapiens
cuauacggccuccuagcuuucc

SEQ ID NO: 791 - RNA, 22 nt, Homo sapiens
aaaaguaauugugguuuuggcc

SEQ ID NO: 792 - RNA, 22 nt, Homo sapiens
ugagaaccacgucugcucugag

SEQ ID NO: 793 - RNA, 23 nt, Homo sapiens
agugccugagggaguaagagccc

SEQ ID NO: 794 - RNA, 19 nt, Homo sapiens
ugucucugcugggguuucu

SEQ ID NO: 795 - RNA, 22 nt, Homo sapiens
aaaaguaauugcgaguuuuacc

SEQ ID NO: 796 - RNA, 22 nt, Homo sapiens
aaaaugguucccuuuagagugu

SEQ ID NO: 797 - RNA, 22 nt, Homo sapiens
agucauuggaggguuugagcag

SEQ ID NO: 798 - RNA, 22 nt, Homo sapiens
aaagugcauccuuuuagagugu

SEQ ID NO: 799 - RNA, 23 nt, Homo sapiens
uucucgaggaaagaagcacuuuc

SEQ ID NO: 800 - RNA, 22 nt, Homo sapiens
cuauacaaucuauugccuuccc

SEQ ID NO: 801 - RNA, 22 nt, Homo sapiens
cuauacagucuacugucuuucc

SEQ ID NO: 802 - RNA, 22 nt, Homo sapiens
caggccauauugugcugccuca

SEQ ID NO: 803 - RNA, 22 nt, Homo sapiens
ccaguauuaacugugcugcuga

SEQ ID NO: 804 - RNA, 22 nt, Homo sapiens
acugcagugaaggcacuuguag

SEQ ID NO: 805 - RNA, 23 nt, Homo sapiens
acugcccuaagugcuccuucugg

SEQ ID NO: 806 - RNA, 22 nt, Homo sapiens
aguuuugcauaguugcacuaca

SEQ ID NO: 807 - RNA, 23 nt, Homo sapiens
aguuuugcagguuugcauccagc

SEQ ID NO: 808 - RNA, 22 nt, Homo sapiens
aguuuugcagguuugcauuuca

SEQ ID NO: 809 - RNA, 22 nt, Homo sapiens
aacaucacagcaagucugugcu

SEQ ID NO: 810 - RNA, 23 nt, Homo sapiens
uaauccuugcuaccugggugaga

SEQ ID NO: 811 - RNA, 22 nt, Homo sapiens
aaaaguaauugcgguuuuugcc

SEQ ID NO: 812 - RNA, 21 nt, Homo sapiens
cacaagguauugguauuaccu

SEQ ID NO: 813 - RNA, 21 nt, Homo sapiens
agggggaaaguucuauagucc

SEQ ID NO: 814 - RNA, 22 nt, Homo sapiens
gacuauagaacuuucccccuca

SEQ ID NO: 815 - RNA, 22 nt, Homo sapiens
augcugacauauuuacuagagg

SEQ ID NO: 816 - RNA, 21 nt, Homo sapiens
ucuaguaagaguggcagucga

SEQ ID NO: 817 - RNA, 22 nt, Homo sapiens
aaugcacccgggcaaggauucu

SEQ ID NO: 818 - RNA, 21 nt, Homo sapiens
uggguuuacguugggagaacu

SEQ ID NO: 819 - RNA, 22 nt, Homo sapiens
acugcauuaugagcacuuaaag

SEQ ID NO: 820 - RNA, 21 nt, Homo sapiens
caacaccagucgaugggcugu

SEQ ID NO: 821 - RNA, 22 nt, Homo sapiens
gggguuccuggggaugggauuu

SEQ ID NO: 822 - RNA, 22 nt, Homo sapiens
ugccuacugagcugauaucagu

SEQ ID NO: 823 - RNA, 22 nt, Homo sapiens
ugccuacugagcugaaacacag

SEQ ID NO: 824 - RNA, 21 nt, Homo sapiens
aggcggagacuugggcaauug

SEQ ID NO: 825 - RNA, 22 nt, Homo sapiens
ccuauucuugguuacuugcacg

SEQ ID NO: 826 - RNA, 22 nt, Homo sapiens
ccuguucuccauuacuuggcuc

SEQ ID NO: 827 - RNA, 22 nt, Homo sapiens
agggcuuagcugcuugugagca

SEQ ID NO: 828 - RNA, 22 nt, Homo sapiens
cacuagauugugagcuccugga

SEQ ID NO: 829 - RNA, 22 nt, Homo sapiens
acugauuucuuuugguguucag

SEQ ID NO: 830 - RNA, 20 nt, Homo sapiens
ucacaccugccucgcccccc

SEQ ID NO: 831 - RNA, 22 nt, Homo sapiens
uuaaugcuaaucgugauagggg

SEQ ID NO: 832 - RNA, 23 nt, Homo sapiens
agcucggucugaggccccucagu

SEQ ID NO: 833 - RNA, 22 nt, Homo sapiens
cugguacaggccugggggacag

SEQ ID NO: 834 - RNA, 21 nt, Homo sapiens
ucgaggagcucacagucuagu

SEQ ID NO: 835 - RNA, 21 nt, Homo sapiens
uggaggagaaggaaggugaug

SEQ ID NO: 836 - RNA, 22 nt, Homo sapiens
aacaauauccuggugcugagug

SEQ ID NO: 837 - RNA, 20 nt, Homo sapiens
auggagauagauauagaaau

SEQ ID NO: 838 - RNA, 21 nt, Homo sapiens
uagauaaaauauugguaccug

SEQ ID NO: 839 - RNA, 20 nt, Homo sapiens
uacaguauagaugauguacu

SEQ ID NO: 840 - RNA, 21 nt, Homo sapiens
uaauuuuauguauaagcuagu

SEQ ID NO: 841 - RNA, 22 nt, Homo sapiens
gcugcgcuuggauuucgucccc

SEQ ID NO: 842 - RNA, 20 nt, Homo sapiens
accaggaggcugaggccccu

SEQ ID NO: 843 - RNA, 22 nt, Homo sapiens
aggugguccguggcgcguucgc

SEQ ID NO: 844 - RNA, 22 nt, Homo sapiens
ggcuacaacacaggacccgggc

SEQ ID NO: 845 - RNA, 22 nt, Homo sapiens
aaagugcuucucuuuggugggu

SEQ ID NO: 846 - RNA, 21 nt, Homo sapiens
ccccaccuccucucuccucag

SEQ ID NO: 847 - RNA, 21 nt, Homo sapiens
uccucuucucccuccucccag

SEQ ID NO: 848 - RNA, 22 nt, Homo sapiens
uucucaaggaggugucguuuau

SEQ ID NO: 849 - RNA, 22 nt, Homo sapiens
uucacaaggaggugucauuuau

SEQ ID NO: 850 - RNA, 26 nt, Homo sapiens
gugagggcaugcaggccuggaugggg

SEQ ID NO: 851 - RNA, 22 nt, Homo sapiens
ccucuuccccuugucucuccag

SEQ ID NO: 852 - RNA, 20 nt, Homo sapiens
gugucugggcggacagcugc

SEQ ID NO: 853 - RNA, 21 nt, Homo sapiens
gugggcgggggcaggugugug

SEQ ID NO: 854 - RNA, 22 nt, Homo sapiens
guggguacggcccagugggggg

SEQ ID NO: 855 - RNA, 21 nt, Homo sapiens
uccuucugcuccgucccccag

SEQ ID NO: 856 - RNA, 22 nt, Homo sapiens
ugagccccugugccgcccccag

SEQ ID NO: 857 - RNA, 20 nt, Homo sapiens
ugagcccuguccucccgcag

SEQ ID NO: 858 - RNA, 20 nt, Homo sapiens
cgugccacccuuuuccccag

SEQ ID NO: 859 - RNA, 21 nt, Homo sapiens
ugcaggaccaagaugagcccu

SEQ ID NO: 860 - RNA, 21 nt, Homo sapiens
caaagguauuugugguuuuug

SEQ ID NO: 861 - RNA, 21 nt, Homo sapiens
aagcauucuuucauugguugg

SEQ ID NO: 862 - RNA, 21 nt, Homo sapiens
uugcucacuguucuucccuag

SEQ ID NO: 863 - RNA, 20 nt, Homo sapiens
ucugcaggguuugcuuugag

SEQ ID NO: 864 - RNA, 22 nt, Homo sapiens
cuuggcaccuagcaagcacuca

SEQ ID NO: 865 - RNA, 24 nt, Homo sapiens
agccugauuaaacacaugcucuga

SEQ ID NO: 866 - RNA, 25 nt, Homo sapiens
gggcgacaaagcaagacucuuucuu

SEQ ID NO: 867 - RNA, 22 nt, Homo sapiens
aaaaguaauugcggucuuuggu

SEQ ID NO: 868 - RNA, 22 nt, Homo sapiens
augguacccuggcauacugagu

SEQ ID NO: 869 - RNA, 22 nt, Homo sapiens
ugugagguuggcauuguugucu

SEQ ID NO: 870 - RNA, 22 nt, Homo sapiens
ucaaaacugaggggcauuuucu

SEQ ID NO: 871 - RNA, 19 nt, Homo sapiens
gaugaugcugcugaugcug

SEQ ID NO: 872 - RNA, 22 nt, Homo sapiens
cuggacugagccgugcuacugg

SEQ ID NO: 873 - RNA, 22 nt, Homo sapiens
caggauguggucaaguguuguu

SEQ ID NO: 874 - RNA, 26 nt, Homo sapiens
aaguaguugguuuguaugagaugguu

SEQ ID NO: 875 - RNA, 22 nt, Homo sapiens
uuuagagacggggucuugcucu

SEQ ID NO: 876 - RNA, 21 nt, Homo sapiens
auauaugaugacuuagcuuuu

SEQ ID NO: 877 - RNA, 17 nt, Homo sapiens
uaauugcuuccauguuu

SEQ ID NO: 878 - RNA, 22 nt, Homo sapiens
uagcaaaaacugcaguuacuuu

SEQ ID NO: 879 - RNA, 23 nt, Homo sapiens
caagucuuauuugagcaccuguu

SEQ ID NO: 880 - RNA, 21 nt, Homo sapiens
agaggauacccuuuguauguu

SEQ ID NO: 881 - RNA, 22 nt, Homo sapiens
cggaugagcaaagaaagugguu

SEQ ID NO: 882 - RNA, 17 nt, Homo sapiens
uaagugcuuccaugcuu

SEQ ID NO: 883 - RNA, 20 nt, Homo sapiens
ucguuugccuuuuucugcuu

SEQ ID NO: 884 - RNA, 23 nt, Homo sapiens
aggaugagcaaagaaaguagauu

SEQ ID NO: 885 - RNA, 23 nt, Homo sapiens
cuggagauauggaagagcugugu

SEQ ID NO: 886 - RNA, 21 nt, Homo sapiens
uaggacacauggucuacuucu

SEQ ID NO: 887 - RNA, 18 nt, Homo sapiens
cagggaggugaaugugau

SEQ ID NO: 888 - RNA, 18 nt, Homo sapiens
ugaggcaguagauugaau

SEQ ID NO: 889 - RNA, 24 nt, Homo sapiens
ccagacagaauucuaugcacuuuc

SEQ ID NO: 890 - RNA, 22 nt, Homo sapiens
aaaaguaaucgcgguuuuuguc

SEQ ID NO: 891 - RNA, 24 nt, Homo sapiens
agccuggaagcuggagccugcagu

SEQ ID NO: 892 - RNA, 22 nt, Homo sapiens
aaaaguacuugcggauuuugcu

SEQ ID NO: 893 - RNA, 21 nt, Homo sapiens
acucuagcugccaaaggcgcu

SEQ ID NO: 894 - RNA, 22 nt, Homo sapiens
ucugggcaacaaagugagaccu

SEQ ID NO: 895 - RNA, 21 nt, Homo sapiens
aagugaucuaaaggccuacau

SEQ ID NO: 896 - RNA, 25 nt, Homo sapiens
ugggaacggguuccggcagacgcug

SEQ ID NO: 897 - RNA, 18 nt, Homo sapiens
ucagcuggcccucauuuc

SEQ ID NO: 898 - RNA, 24 nt, Homo sapiens
uugcagcugccugggagugacuuc

SEQ ID NO: 899 - RNA, 22 nt, Homo sapiens
ugcuggaucagugguucgaguc

SEQ ID NO: 900 - RNA, 22 nt, Homo sapiens
cuccugagccauucugagccuc

SEQ ID NO: 901 - RNA, 23 nt, Homo sapiens
gagggucuugggagggaugugac

SEQ ID NO: 902 - RNA, 21 nt, Homo sapiens
uggacugcccugaucuggaga

SEQ ID NO: 903 - RNA, 17 nt, Homo sapiens
ucccaccgcugccaccc

SEQ ID NO: 904 - RNA, 24 nt, Homo sapiens
uggcccugacugaagaccagcagu

SEQ ID NO: 905 - RNA, 17 nt, Homo sapiens
gugggggagaggcuguc

SEQ ID NO: 906 - RNA, 27 nt, Homo sapiens
cacuguaggugauggugagagugggca

SEQ ID NO: 907 - RNA, 23 nt, Homo sapiens
ccugcagcgacuugauggcuucc

SEQ ID NO: 908 - RNA, 20 nt, Homo sapiens
uaaagagcccuguggagaca

SEQ ID NO: 909 - RNA, 22 nt, Homo sapiens
aaaagcuggguugagagggcaa

SEQ ID NO: 910 - RNA, 26 nt, Homo sapiens
gaugaugauggcagcaaauucugaaa

SEQ ID NO: 911 - RNA, 22 nt, Homo sapiens
uuuccggcucgcgugggugugu

SEQ ID NO: 912 - RNA, 22 nt, Homo sapiens
aggcauugacuucucacuagcu

SEQ ID NO: 913 - RNA, 22 nt, Homo sapiens
uaguacugugcauaucaucuau

SEQ ID NO: 914 - RNA, 22 nt, Homo sapiens
augggugaauuuguagaaggau

SEQ ID NO: 915 - RNA, 22 nt, Homo sapiens
aacuggaucaauuauaggagug

SEQ ID NO: 916 - RNA, 22 nt, Homo sapiens
gguggcccggccgugccugagg

SEQ ID NO: 917 - RNA, 21 nt, Homo sapiens
gugccagcugcagugggggag

SEQ ID NO: 918 - RNA, 22 nt, Homo sapiens
agaaggaaauugaauucauuua

SEQ ID NO: 919 - RNA, 22 nt, Homo sapiens
uucauucggcuguccagaugua

SEQ ID NO: 920 - RNA, 21 nt, Homo sapiens
uuaggccgcagaucuggguga

SEQ ID NO: 921 - RNA, 19 nt, Homo sapiens
uggauuuuuggaucaggga

SEQ ID NO: 922 - RNA, 22 nt, Homo sapiens
uuuucaacucuaaugggagaga

SEQ ID NO: 923 - RNA, 22 nt, Homo sapiens
acgcccuucccccccuucuuca

SEQ ID NO: 924 - RNA, 27 nt, Homo sapiens
accuucuuguauaagcacugugcuaaa

SEQ ID NO: 925 - RNA, 23 nt, Homo sapiens
uggaguccaggaaucugcauuuu

SEQ ID NO: 926 - RNA, 21 nt, Homo sapiens
ucguggccuggucuccauuau

SEQ ID NO: 927 - RNA, 27 nt, Homo sapiens
auugaucaucgacacuucgaacgcaau

SEQ ID NO: 928 - RNA, 22 nt, Homo sapiens
uuugaggcuacagugagaugug

SEQ ID NO: 929 - RNA, 18 nt, Homo sapiens
gcaugggugguucagugg

SEQ ID NO: 930 - RNA, 21 nt, Homo sapiens
cccggagccaggaugcagcuc

SEQ ID NO: 931 - RNA, 21 nt, Homo sapiens
uguucauguagauguuuaagc

SEQ ID NO: 932 - RNA, 22 nt, Homo sapiens
aaaacuguaauuacuuuuguac

SEQ ID NO: 933 - RNA, 20 nt, Homo sapiens
ucacuguucagacaggcgga

SEQ ID NO: 934 - RNA, 22 nt, Homo sapiens
aaaaacugagacuacuuuugca

SEQ ID NO: 935 - RNA, 18 nt, Homo sapiens
gucccuguucaggcgcca

SEQ ID NO: 936 - RNA, 17 nt, Homo sapiens
ucccuguucgggcgcca

SEQ ID NO: 937 - RNA, 21 nt, Homo sapiens
ccuguugaaguguaaucccca

SEQ ID NO: 938 - RNA, 21 nt, Homo sapiens
acggugcuggauguggccuuu

SEQ ID NO: 939 - RNA, 22 nt, Homo sapiens
caaaaguaauuguggauuuugu

SEQ ID NO: 940 - RNA, 22 nt, Homo sapiens
ucuacaaaggaaagcgcuuucu

SEQ ID NO: 941 - RNA, 22 nt, Homo sapiens
acccgucccguucguccccgga

SEQ ID NO: 942 - RNA, 21 nt, Homo sapiens
agagaagaagaucagccugca

SEQ ID NO: 943 - RNA, 17 nt, Homo sapiens
ucucgcuggggccucca

SEQ ID NO: 944 - RNA, 18 nt, Homo sapiens
aucccaccucugccacca

SEQ ID NO: 945 - RNA, 23 nt, Homo sapiens
uauucauuuauccccagccuaca

SEQ ID NO: 946 - RNA, 21 nt, Homo sapiens
uugggacauacuuaugcuaaa

SEQ ID NO: 947 - RNA, 18 nt, Homo sapiens
uugagaaggaggcugcug

SEQ ID NO: 948 - RNA, 22 nt, Homo sapiens
ucuauacagacccuggcuuuuc

SEQ ID NO: 949 - RNA, 22 nt, Homo sapiens
aaaaguauuugcggguuuuguc

SEQ ID NO: 950 - RNA, 22 nt, Homo sapiens
uggguggucuggagauuugugc

SEQ ID NO: 951 - RNA, 18 nt, Homo sapiens
uccagugcccuccucucc

SEQ ID NO: 952 - RNA, 22 nt, Homo sapiens
uuagggcccuggcuccaucucc

SEQ ID NO: 953 - RNA, 22 nt, Homo sapiens
aaaaguaauugcggauuuugcc

SEQ ID NO: 954 - RNA, 21 nt, Homo sapiens
agugaaugauggguucugacc

SEQ ID NO: 955 - RNA, 21 nt, Homo sapiens
ucacagugaaccggucucuuu

SEQ ID NO: 956 - RNA, 21 nt, Homo sapiens
ucacagugaaccggucucuuu

SEQ ID NO: 957 - RNA, 22 nt, Homo sapiens
aaggagcucacagucuauugag

SEQ ID NO: 958 - RNA, 23 nt, Homo sapiens
ugauuguagccuuuuggaguaga

SEQ ID NO: 959 - RNA, 22 nt, Homo sapiens
aguggggaacccuuccaugagg

SEQ ID NO: 960 - RNA, 24 nt, Homo sapiens
aauccuuggaaccuaggugugagu

SEQ ID NO: 961 - RNA, 22 nt, Homo sapiens
uuauaauacaaccugauaagug

SEQ ID NO: 962 - RNA, 22 nt, Homo sapiens
auauaauacaaccugcuaagug

SEQ ID NO: 963 - RNA, 22 nt, Homo sapiens
auaauacaaccugcuaagugcu

SEQ ID NO: 964 - RNA, 23 nt, Homo sapiens
cccaguguucagacuaccuguuc

SEQ ID NO: 965 - RNA, 22 nt, Homo sapiens
acaguagucugcacauugguua

SEQ ID NO: 966 - RNA, 23 nt, Homo sapiens
cccaguguucagacuaccuguuc

SEQ ID NO: 967 - RNA, 22 nt, Homo sapiens
acaguagucugcacauugguua

SEQ ID NO: 968 - RNA, 21 nt, Homo sapiens
auccuugcuaucugggugcua

SEQ ID NO: 969 - RNA, 22 nt, Homo sapiens
aaugcaccugggcaaggauuca

SEQ ID NO: 970 - RNA, 22 nt, Homo sapiens
uggcaguguauuguuagcuggu

SEQ ID NO: 971 - RNA, 22 nt, Homo sapiens
aggcaguguauuguuagcuggc

SEQ ID NO: 972 - RNA, 22 nt, Homo sapiens
cagccacaacuacccugccacu

SEQ ID NO: 973 - RNA, 25 nt, Homo sapiens
uaggcaguguauugcuagcggcugu

SEQ ID NO: 974 - RNA, 23 nt, Homo sapiens
uugcuaguugcacuccucucugu

SEQ ID NO: 975 - RNA, 22 nt, Homo sapiens
cucuagagggaagcacuuucug

SEQ ID NO: 976 - RNA, 21 nt, Homo sapiens
caaagcgcuucccuuuggagc

SEQ ID NO: 977 - RNA, 22 nt, Homo sapiens
uaugugccuuuggacuacaucg

SEQ ID NO: 978 - RNA, 21 nt, Homo sapiens
gcaguccaugggcauauacac

SEQ ID NO: 979 - RNA, 22 nt, Homo sapiens
ucucugggccugugucuuaggc

SEQ ID NO: 980 - RNA, 23 nt, Homo sapiens
gcaaagcacacggccugcagaga

SEQ ID NO: 981 - RNA, 22 nt, Homo sapiens
cugaagcucagagggcucugau

SEQ ID NO: 982 - RNA, 22 nt, Homo sapiens
ucggauccgucugagcuuggcu

SEQ ID NO: 983 - RNA, 22 nt, Homo sapiens
uuauaauacaaccugauaagug

SEQ ID NO: 984 - RNA, 22 nt, Homo sapiens
cuuaucagauuguauuguaauu

SEQ ID NO: 985 - RNA, 22 nt, Homo sapiens
auauaauacaaccugcuaagug

SEQ ID NO: 986 - RNA, 22 nt, Homo sapiens
cuuagcagguuguauuaucauu

SEQ ID NO: 987 - RNA, 22 nt, Homo sapiens
auaauacaaccugcuaagugcu

SEQ ID NO: 988 - RNA, 23 nt, Homo sapiens
cagugcaauaguauugucaaagc

SEQ ID NO: 989 - RNA, 23 nt, Homo sapiens
cagugcaaugauauugucaaagc

SEQ ID NO: 990 - RNA, 22 nt, Homo sapiens
caugccuugaguguaggaccgu

SEQ ID NO: 991 - RNA, 22 nt, Homo sapiens
ccucccacacccaaggcuugca

SEQ ID NO: 992 - RNA, 22 nt, Homo sapiens
uccuguacugagcugccccgag

SEQ ID NO: 993 - RNA, 21 nt, Homo sapiens
cggggcagcucaguacaggau

SEQ ID NO: 994 - RNA, 22 nt, Homo sapiens
cagugguuuuacccuaugguag

SEQ ID NO: 995 - RNA, 21 nt, Homo sapiens
uaccacaggguagaaccacgg

SEQ ID NO: 996 - RNA, 22 nt, Homo sapiens
uggcagugucuuagcugguugu

SEQ ID NO: 997 - RNA, 22 nt, Homo sapiens
caaucagcaaguauacugcccu

SEQ ID NO: 998 - RNA, 22 nt, Homo sapiens
caaucacuaacuccacugccau

SEQ ID NO: 999 - RNA, 23 nt, Homo sapiens
uaggcagugucauuagcugauug

SEQ ID NO: 1000 - RNA, 23 nt, Homo sapiens
aggcaguguaguuagcugauugc

(19) United States
(12) Patent Application Publication
(10) Pub. No.: US 2012/0219958 A1
(43) Pub. Date: Aug. 30, 2012

(54) MicroRNA Signatures Differentiating Uterine and Ovarian Papillary Serous Tumors

(75) Inventor: Joanne B. Weidhaas, Westport, CT (US)

(73) Assignee: Yale University, New Haven, CT (US)

(21) Appl. No.: 13/505,584

(22) PCT Filed: Nov. 9, 2010
(86) PCT No.: PCT/US10/56091

§ 371 Date: May 2, 2012

(51) Int. Cl. C12Q 1/68; C07H 21/02

(52) U.S. Cl. 435/6.12; 435/6.1; 536/24.5

(57) ABSTRACT

The invention provides a papillary serous miRNA signature and methods for determining the identity, origin, and stage, of concurrent endometrial and ovarian papillary serous tumors. Exemplary origins of concurrent endometrial and ovarian tumors include, but are not limited to, the uterus, ovary, fallopian tubes, and peritoneum.

### RELATED APPLICATIONS

This application claims the benefit of provisional application U.S. Ser. No. 61/259,601, filed Nov. 9, 2009, the contents of which are herein incorporated by reference in their entirety.

### INCORPORATION OF SEQUENCE LISTING

The contents of the text file named “34592508001WOSeqList.txt,” which was created on Nov. 9, 2010 and is 147 KB in size, are hereby incorporated by reference in their entirety.

### FIELD OF THE INVENTION

This invention relates generally to the fields of cancer and molecular biology. The invention provides methods for determining the identity and stage of concurrent tumors of the same subtype and unknown origin.

### BACKGROUND OF THE INVENTION

Papillary serous cancer of the ovary and uterus look identical pathologically. This poses a problem because it is not uncommon for papillary serous cancer to be present in the ovary and the uterus simultaneously. Importantly, if a patient has two separate papillary serous cancers, versus a cancer that has started in the ovary and spread to the uterus, or that has started in the uterus and spread to the ovary, the patient's stage of disease, and, thus the patient's treatment is significantly different. If a patient has two primary cancers, treatment can likely stop after surgery. If a patient instead has metastatic cancer from one organ to the other, the addition of chemotherapy is critical. Because there is no pathological means to determine which scenario is correct, e.g. two primary tumors versus the presence of at least one metastatic cancer, many patients are over-treated, and, even worse, some patients are under-treated. In addition, depending on the organ of origin of the tumor, chemotherapy regimens are different. The ability to determine a patient's true stage and, consequently, the patient's correct treatment requires an ability to reliably differentiate papillary serous cancers of the ovary from papillary serous cancers of the uterus.

Histologic differentiation of serous tumors of gynecologic origin is a challenging problem to be solved. When patients are found to have two tumors, problems invariably arise as to whether these tumors represent primary tumors that have arisen independently or metastases of a single primary tumor. Many pathologic and histologic approaches have been described, but despite extensive efforts, a need still remains for an accurate method of determining the origin and synchronicity of these concurrent tumors. Such a classification is clinically pertinent, affecting the patient's diagnosis, prognosis, treatment and disease management. The invention provides compositions and methods to solve this long-felt need in the art.

### SUMMARY OF THE INVENTION

MiRNA signatures and methods of the invention demonstrate that miRNA analysis reliably differentiates between papillary serous carcinomas of uterine and ovarian origins. This signature is critically important because these subtypes appear to be identical and cannot be distinguished by any known method. As such, without the use of this miRNA signature to determine the origins of concurrent tumors, an accurate diagnosis cannot be made and the patient's prognosis is uncertain.

Specifically, the invention provides a microRNA signature comprising one or more miRNAs selected from the group consisting of hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11), wherein the increased expression of these miRNAs in a uterine versus an ovarian cancer cell indicates that the cancer cell is a uterine cell. In alternative embodiments, the invention provides a microRNA signature comprising two, three, four, five, six, seven, eight, nine, or ten or more miRNAs selected from the group consisting of hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11), wherein the increased expression of these miRNAs in a uterine versus an ovarian cancer cell indicates that the cancer cell is a uterine cell.

Alternatively, the invention provides a microRNA signature comprising hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11), wherein the increased expression of these miRNAs in a uterine versus an ovarian cancer cell indicates that the cancer cell is a uterine cell.

The invention further provides a microRNA signature comprising one or more of the miRNAs selected from the group consisting of hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, and hsa-miR-202, wherein a statistically significant change in the expression of any one of these miRNAs in a uterine versus ovarian cancer cell indicates that the cancer cell is a uterine cell. Optionally, this microRNA signature further comprises one or more of the miRNAs selected from the group consisting of hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, and hsa-miR-484. Alternatively, this microRNA signature also comprises one or more of the miRNAs selected from the group consisting of hsa-miR-629, hsa-miR-193b, hsa-miR-885-5p, hsa-miR-155, hsa-miR-200b, hsa-miR-493, hsa-miR-148a, and hsa-miR-101. In certain embodiments, this microRNA signature also comprises one or more of the miRNAs selected from the group consisting of hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, and hsa-miR-362-5p. The statistically significant change in the expression of any one of these miRNAs is alternatively an increase or a decrease.

The miRNA signatures provided herein are determined for specific cell types, including, but not limited to, a cancer cell residing in the uterus, ovary, fallopian tube, or peritoneum. Preferably the cancer cell is the papillary serous subtype.

In certain embodiments, the invention provides an amplified microRNA signature. The term “amplified” describes a process by which the miRNA is detected or the expression level of a miRNA determined. The amplification of a miRNA may result in the generation of one or more copies of a complementary DNA or RNA sequence. This complementary DNA or RNA sequence may be detected by means that would further amplify a detectable signal, e.g. a fluorescent signal. Alternatively, a complementary DNA or RNA sequence may be may be used as probe or primer for hybridization or sequencing methods.

In a preferred embodiment, total RNA is extracted from tumor cells of papillary serous carcinoma tumors of distinct tumors, reverse transcribed into cDNA, and amplified by real-time polymerase chain reaction (PCR). The resultant miRNA profile is normalized to a control RNA from the same sample, which, optionally, also has been extracted, reverse transcribed into cDNA and amplified by real-time polymerase chain reaction (PCR). Normalized miRNA profiles are compared between papillary serous carcinoma tumors from distinct origins to generate a miRNA signature.

Alternatively, or in addition, the term “amplified” describes a hybridization process by the expression levels of miRNAs in a cancer cell determined. For example, complementary sequences to those provided in Table 2, which include the miRNAs listed in Tables 4 and 5, are used as probes to specifically target miRNAs expressed in a cancer cell. A complementary RNA or DNA sequence is readily determined by matching each adenine nucleobase in the miRNA (when read in the 5′ to 3′ orientation) with either a uracil (RNA) or thymine (DNA) nucleobase in the complementary sequence, each cytosine nucleobase in the miRNA with a guanine nucleobase in the complementary sequence, each guanine nucleobase in the miRNA with a cytosine nucleobase in the complementary sequence, and each thymine with an adenine nucleobase in the complementary sequence. Probes of the invention comprise, consist essentially of, or consist of a sequence complementary to, for example, but not limited to, the miRNAs provided in Table 2. Probes are optionally amplified using a polymerase chain reaction to increase abundance and facilitate detection. Alternatively, probes are labeled with a fluorescent tag, and the signal from the tag is amplified by application of, for instance, a primary and labeled secondary antibody.

Moreover, the term “amplified” describes a sequencing process by the expression levels of miRNAs in a cancer cell determined. High throughput sequencing methods employ primers and polymerization reactions to incorporate labeled nucleotides. These methods could be used quantitatively to determine the relative levels of a miRNA in a cancer cell.

All methods that isolate, purify, clone, duplicate, copy, sort, label, amplify, or manipulate the miRNA sequence, or which involve the use of a DNA or RNA molecule complementary to the miRNA are contemplated.

The invention provides a method for determining the origin of a papillary serous carcinoma tumor, the method comprising detecting the miRNA expression profile of a sample from the papillary serous carcinoma tumor and comparing it to an miRNA expression profile of a sample from a uterine tumor or an ovarian tumor, thereby to identify the origin of the papillary serous carcinoma tumor.

In certain embodiments of this method, the miRNA expression profile comprises a statistically significant change in the expression of one or more of hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, or hsa-miR-202 in a uterine versus ovarian cancer cell.

Optionally, the miRNA expression profile further comprises a statistically significant change in the expression of one or more of one or more of hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, or hsa-miR-484 in a uterine versus ovarian cancer cell.

Optionally, the miRNA expression profile further comprises a statistically significant change in the expression of one or more of one or more of hsa-miR-629, hsa-miR-193b, hsa-miR-885-5p, hsa-miR-155, hsa-miR-200b, hsa-miR-493, hsa-miR-148a, or hsa-miR-101 in a uterine versus ovarian cancer cell.

Optionally, the miRNA expression profile further comprises a statistically significant change in the expression of one or more of one or more of hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, or hsa-miR-362-5p in a uterine versus ovarian cancer cell.

The statistically significant change is alternatively an increase or a decrease.

In an alternative embodiment of this method, the miRNA expression profile comprises the increased expression one or more of hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11) in a uterine versus an ovarian cancer cell.

Moreover, the invention provides a method of generating an miRNA signature that distinguishes between at least two papillary serous carcinoma tumors of distinct origin, including the steps of: (a) obtaining a sample of at least a first and second papillary serous carcinoma tumor; (b) extracting total RNA of said first and second samples; (c) determining a miRNA expression profile of said first and second samples; and (d) comparing the miRNA expression profiles of said first and second samples, wherein a plurality of statistically-significant differences identified between the miRNA expression profiles of the first and second miRNA expression profiles identifies a miRNA signature that distinguishes between the first and second papillary serous carcinoma tumors. Optionally, the method further includes amplifying at least one miRNA from said first and second samples following the extracting step. The determining step further includes normalizing at least one miRNA expression level of at least one miRNA from the first or second tumor sample to a control RNA. In one aspect, the plurality comprises between 2-30 statistically significant differences. The term, “statistically significantly different” is meant to describe a statistical difference having a p-value of less than 0.1, and preferably less than 0.05. Most preferably, the statistical difference has a p-value of less than 0.01.

In certain embodiments of this method, the papillary serous carcinoma tumor resides in the uterus, ovary, fallopian tube, omentum, or peritoneum. In other aspects, the first or second papillary serous carcinoma tumor is a uterine papillary serous carcinoma tumor. Moreover, the first or second papillary serous carcinoma tumor is an ovarian papillary serous carcinoma tumor.

Exemplary control RNAs of this method include, but are not limited to, non-coding RNA selected from the group consisting of transfer RNA (tRNA), small nuclear RNA (snRNA) and small nucleolar RNA (snoRNA). In certain embodiments, the control RNA is a non-coding RNA of between 45 and 200 nucleotides. Alternatively, or in addition, the control RNA is highly- and invariably-expressed between the first and second papillary serous tumor.

The invention further provides a method of determining the origin of a papillary serous carcinoma tumor, including the steps of: (a) obtaining a sample of a papillary serous carcinoma tumor; (b) extracting total RNA of the sample; (c) determining an miRNA expression profile of the sample; and (d) comparing the miRNA expression profile of the tumor sample to a papillary serous miRNA signature described herein, wherein replication of the miRNA signature within the miRNA expression profile of the tumor sample indicates that the cells of the tumor sample are uterine cells. Optionally, the method further includes amplifying at least one miRNA from the sample. The determining step further includes normalizing at least one miRNA expression level of at least one miRNA from the tumor sample to a control RNA. Exemplary control RNAs include, but are not limited to, RNU44 (SEQ ID NO: 12) and RNU48 (SEQ ID NO: 13), or any other control RNA.

According to this method, the papillary serous carcinoma tumor resides in, for example, the uterus, ovary, fallopian tube, or peritoneum.

The invention also provides a method of determining the stage of concurrent uterine and ovarian papillary serous carcinoma tumors from a patient, including the steps of: (a) obtaining a sample of a uterine tumor and an ovarian tumor; (b) extracting total RNA of said uterine sample and said ovarian sample; (c) determining a miRNA expression profile of the uterine sample and the ovarian sample; and (d) comparing the miRNA expression profiles of the uterine sample and the ovarian sample to a papillary serous miRNA signature described herein, wherein replication of the papillary serous miRNA signature within the miRNA expression profile of the uterine sample, but not the ovarian sample, indicates that the uterine and the ovarian tumors are synchronous primary tumors, thereby determining that the tumors are stage I or lower. Optionally, the method further includes amplifying at least one miRNA from the uterine sample and the ovarian sample. Alternatively, this method determines that the patient has a lower stage of cancer than if the tumors had spread from one organ to the other.

Moreover, the invention provides a method of determining the stage of concurrent uterine and ovarian papillary serous carcinoma tumors from a patient, including the steps of (a) obtaining a sample of a uterine tumor and an ovarian tumor; (b) extracting total RNA of the uterine sample and the ovarian sample; (c) determining a miRNA expression profile of the uterine sample and the ovarian sample; and (d) comparing the miRNA expression profiles of the uterine sample and the ovarian sample to a papillary serous miRNA signature described herein, wherein replication of the papillary serous miRNA signature within the miRNA expression profile of both the uterine and ovarian samples indicates that the uterine tumor is a primary tumor and the ovarian tumor is a metastasis from the uterus, thereby determining that the tumors are stage III or higher. Optionally, the method further includes amplifying at least one miRNA from the uterine sample and the ovarian sample. Alternatively, this method determines that the patient has a higher stage cancer, stage III or higher.

Furthermore, the invention provides a method of determining the stage of concurrent uterine and ovarian papillary serous carcinoma tumors from a patient, including the steps of: (a) obtaining a sample of a uterine tumor and an ovarian tumor; (b) extracting total RNA of the uterine sample and the ovarian sample; (c) determining a miRNA expression profile of the uterine sample and the ovarian sample; and (d) comparing the miRNA expression profiles of the uterine sample and the ovarian sample to a papillary serous miRNA signature described herein, wherein absence of the papillary serous miRNA signature within the miRNA expression profile of either the uterine and ovarian samples indicates that the ovarian tumor is a primary tumor and the uterine tumor is a metastasis from the ovary, thereby determining that the tumors are stage II or higher. Optionally, the method further includes amplifying at least one miRNA from the uterine sample and the ovarian sample. Alternatively, this method determines that the patient has metastatic disease and cancer of a higher stage, i.e., stage II or higher.

In certain embodiments of the methods described herein, cancer stage is determined according to the TNM system. Alternatively, cancer stage is determined according to the FIGO system.

In certain aspects of the methods described herein, the UPSC miRNA is the microRNA signature includes hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), hsa-miR-223 (SEQ ID NO: 11), wherein the increased expression of these miRNAs in a cancer cell indicates that the cancer cell is a uterine cell. Optionally, this signature is an amplified microRNA signature.

Alternatively, the UPSC miRNA is the microRNA signature includes one or more of the miRNAs selected from the group consisting of hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, and hsa-miR-202, wherein a statistically significant change in the expression of any one of these miRNAs in a uterine versus ovarian cancer cell indicates that the cancer cell is a uterine cell. Optionally, this amplified microRNA signature further comprises one or more of the miRNAs selected from the group consisting of hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, and hsa-miR-484. Alternatively, this amplified microRNA signature also comprises one or more of the miRNAs selected from the group consisting of hsa-miR-629, hsa-miR-193b, hsa-miR-885-5p, hsa-miR-155, hsa-miR-200b, hsa-miR-493, hsa-miR-148a, and hsa-miR-101. In certain embodiments, this amplified microRNA signature also comprises one or more of the miRNAs selected from the group consisting of hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, and hsa-miR-362-5p. Optionally, this signature is an amplified microRNA signature.

### BRIEF DESCRIPTION OF THE DRAWINGS

FIG. 1 is a schematic representation of the biogenesis of miRNAs.

FIG. 2 is a graphical representation of a miRNA expression signature that discriminates between papillary serous cancers of uterine and ovarian origin. A shading key and histogram is provided in the upper left corner. The upper side of the figure displays a tree relating patients by expression patterns.

FIG. 3 is a graphical representation of a miRNA expression signature that discriminates between papillary serous cancers of uterine and ovarian origin. MiRNA expression was analyzed by miRNA array from samples taken from a patient having tumors concurrently present in both the uterus and ovary (two samples with asterisks). These samples clustered within the uterine miRNA signatures (wherein expression of each miRNA generally increases upon moving from left to right on the diagram), indicating that both tumors had a uterine origin (see Example 3).

### DETAILED DESCRIPTION

Synchronous endometrial and ovarian malignancies occur in 5% of women presenting with endometrial cancer and 10% of the patients presenting with ovarian cancer. When the histology of both sites is papillary serous, correct diagnosis is exceedingly challenging for the clinicians and pathologists. This pathologic differentiation is critical as it influences cancer staging, adjuvant therapy, and prognosis. Previous studies found that the prognosis of synchronous primary cancers of the endometrium and ovary, in low grade and stage, is favorable, and differs greatly from much higher stage of metastatic disease of a single organ.

MicroRNAs (miRNAs) are a recently-discovered class of 22-nucleotide noncoding RNAs, which globally regulate gene expression by selectively inhibiting gene expression of targeted mRNA transcripts at the post-transcriptional level. MiRNAs are universally misexpressed in virtually all human cancer types. Thus, miRNAs may function as a novel class of oncogene or tumor suppressor gene.

Furthermore, miRNAs have been shown to be able to differentiate adenocarcinomas of unknown origin with identical histology. For this cancer subtype, microRNA signatures are unique for each tissue type. As such, adenocarcinomas of unknown origin can be classified by their starting tissue type by microRNA signature in 16/17 cases, where gene expression profiling (which has been the only thing possible previously) can only correctly classify cancers 2/17 times.

A superior property of the instant invention is the ability to differentiate carcinomas of ovarian or uterine origin that, like the adenocarcinomas discussed above, appear otherwise identical by pathological analyses (including molecular and histological studies) but also are near to each other spatially and frequently spread to each other. Specifically, the invention provides a method for differentiating cancers of the same subtype, papillary serous carcinoma, but different origin, uterine versus ovarian, based upon expression levels of a defined group of miRNAs, the papillary serous miRNA signature. Core biopsies were obtained of cases that were confined only in the ovary or only in the uterus. Analyses of the differential miRNA expression in these samples produced a statistically significant microRNA signature that clearly separates papillary serous cancer of the ovary from papillary serous cancer of the uterus. This microRNA signature determines the origins of concurrent tumors in patients presenting with papillary serous cancer in the ovary and the uterus and significantly impacts treatment recommendations, as well as prognosis prediction for these patients.

Previously it has been impossible to differentiate papillary serous cancers of the ovary and uterus, which was a significant diagnostic dilemma in cases where they were found in both organs. MicroRNAs are only recently discovered, and this is the first evidence that they can be used to identify papillary serous cancer in such similar organs. The papillary serous microRNA signature can be applied to immediately guide treatment decisions.

### Cancer

Cancer is a group of many related diseases. All cancers begin in cells that make up the organs of the body. Normally, cells division is a regulated process throughout development and adulthood. Cells are instructed to grow and divide to form new cells only as the body needs them. For instance, when existing cells die, new cells are generated to replace them.

When cell division or cell proliferation becomes unregulated or misregulated, new cells form even when the body does not need them. Alternatively, or in addition, the lives of existing cells are prolonged because they do not engage in programmed cell death at the expected times. Tumors result from the resultant accumulation of cells that forms when cell proliferation and/or death becomes misregulated.

The term “tumor” is meant to describe an abnormal growth of body tissue resulting from a cell proliferative disorder, which is benign (non-cancerous), pre-malignant (pre-cancerous) or malignant (cancerous). Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell,” is defined as any cell that divides at a rate that exceeds, or is greater than, what is expected or observed among neighboring or juxtaposed cells within the same tissue.

Cancer cells can invade and damage nearby tissues and organs when they detach from the primary malignant tumor, enter the bloodstream or lymphatic system, and form new tumors in other organs. The spread of cancer is called metastasis. In the case of uterine, ovarian, fallopian tube and primary peritoneal cancers, these frequently spread form one organ to the next, and indicate a higher stage, while not necessarily a stage IV cancer. Regardless, the spread from one organ to the next indicates a higher stage and a worse prognosis compared to synchronous small primary tumors arising independently in each organ. Cancers that are distinguished using the miRNA signatures and methods of the invention include, but are not limited to, papillary serous carcinomas of the uterus, ovary, fallopian tubes, and peritoneum.

A subject of the invention is preferably a mammal. The mammal can be a human, non-human primate, mouse, rat, dog, cat, horse, or cow, but are not limited to these examples. Mammals other than humans can be advantageously used as subjects that represent animal models of a particular disease. A subject can be male or female. A subject can be one who has been previously diagnosed or identified as having a disease and optionally has already undergone, or is undergoing, a therapeutic intervention for the disease. Alternatively, a subject can also be one who has not been previously diagnosed as having the disease. For example, a subject can be one who exhibits one or more risk factors for a disease. A subject is also a patient.

The biological or tumor sample can be any tissue or fluid that contains a nucleic acid. Various embodiments include paraffin imbedded tissue, frozen tissue, surgical fine needle aspirations, cells of the uterus, ovary, skin, muscle, lung, head and neck, esophagus, kidney, pancreas, mouth, throat, pharynx, larynx, esophagus, facia, brain, prostate, breast, endometrium, small intestine, blood cells, liver, testes, ovaries, uterus, cervix, colon, stomach, spleen, lymph node, or bone marrow. Other embodiments include fluid samples such as bronchial brushes, bronchial washes, bronchial ravages, peripheral blood lymphocytes, lymph fluid, ascites, serous fluid, pleural effusion, sputum, cerebrospinal fluid, lacrimal fluid, esophageal washes, and stool or urinary specimens such as bladder washing and urine.

In certain embodiments, the papillary serous miRNA signature and methods of the invention determines the true stage of one or more concurrent papillary serous carcinomas. The true stage is the most critical factor for providing an accurate diagnosis, and therefore, providing an accurate prognosis. The true stage of a cancer determines the course of treatment prescribed to a subject or patient. For instance, in situ and primary tumors are staged 0 and 1-3, respectively, whereas, metastasized cancer is stage IV, as described below. Thus, the papillary serous miRNA signature and methods of the invention further determine the severity of cancer, because higher stage cancer is more severe than lower stage cancers.

The term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes).

The cancer stage which is present at diagnosis is the single-most important indicator of patient prognosis and survival. As such, patient treatment regimens are typically designed in response to the determination of cancer stage made at the time of diagnosis. Cancer staging is generally performed according to the Tumor, Node, Metastasis (TNM) System, which is the universally-accepted system of the Union Internationale Centre le Cancer (UICC) and the American Joint Committee on Cancer (AJCC). FIGO (Fédération Internationale de Gynécologie et Obstétrique, International Federation of Gynecology and Obstetrics) is an international organization that defines staging systems in gynecological malignancy.

The TNM categories correspond with the FIGO staging system. The TNM system further denotes the stage of the cancer as either “clinical stage,” or “pathological stage.” The clinical stage, denoted by a “c” preceding the grade, is based upon all of the information obtainable prior to surgery including physical examination of the patient, radiologic examination, and endoscopy. Moreover, the pathological stage, denoted by a lower case “p” preceding the grade, is based upon all of the information gathered prior to surgery as well as additional information gained by pathological microscopic examination of the tumor. Although biopsy is used to remove tissue and perform clinical and pathological studies, surgical removal of the tumor is preferred. Biopsy can be performed according to a variety of methods, including, but not limited to, fine needle aspiration, core biopsy, and excision biopsy. Furthermore, this system includes a C-factor, or certainty factor, that reflects the validity of classification with respect to the diagnostic methods employed.

Overall Stage Grouping is also referred to as Roman Numeral Staging. This system uses numerals I, II, III, and IV (plus the 0) to describe the progression of cancer. Stage 0 is in situ carcinoma, a pre-invasive malignancy that does not invade the basement membrane and by definition does not metastasize. Stages I-III indicate increasingly severe conditions with increasing poor prognoses. Higher numbers indicate more extensive disease: greater tumor size, and/or spread of the cancer to nearby lymph nodes, and/or organs adjacent to the primary tumor. Typically, stage IV is metastatic cancer indicating that the cancer has spread to another distant organ. However, spread of a papillary serious cancer to the uterus from the ovary or the ovary from the uterus is stage II or III, respectively.

Within the TNM system, a cancer may also be designated as recurrent, meaning that it has appeared again after being in remission or after all visible tumor has been eliminated. Recurrence can either be local, meaning that it appears in the same location as the original, or distant, meaning that it appears in a different part of the body.

The TNM system has more specific grades including the following primary tumor (T) grades: TX=Primary tumor cannot be evaluated, T0=No evidence of primary tumor, Tis=In situ carcinoma in situ, and T1-T4=increasing size and/or extent of the primary tumor. The TNM system further includes the following specific regional lymph node grades: NX=Regional lymph nodes (N) cannot be evaluated, N0=No regional lymph node involvement (no cancer found in the lymph nodes), and N1-N3=Increasing involvement of regional lymph nodes (number and/or extent of spread). Furthermore, the TNM system includes the following distant metastasis (M) grades: MX=Distant metastasis cannot be evaluated, M0=No distant metastasis (cancer has not spread to other parts of the body), and M1=Distant metastasis (cancer has spread to distant parts of the body).

As described herein, the FIGO system of grading gynecological tumors corresponds to the TNM system. The main goal of staging cancer is to determine the extent of the disease. Similar to the TNM system, factors used to stage cancer in the FIGO system include the depth of the tumor, whether the tumor has spread to the cervix and other nearby organs, the cytology of the cancer (cellular make-up and activity), whether it has metastasized to the lymph nodes, and the extent to which it has spread to other parts of the body. The FIGO system is summarized below in Table 1A. Endometrial cancer in patients who are unable to undergo surgical evaluation is staged using an older, clinical staging system provided in Table 1B.

TABLE 1A
FIGO Surgical Stages For Endometrial Cancer

| | |
|---|---|
| Stage I | The tumor is confined to the uterine fundus. |
| Stage IA | The tumor is limited to the endometrium. |
| Stage IB | The tumor invades less than one-half of the |
| | myometrium. |
| Stage IC | The tumor invades more than one-half of the |
| | myometrium. |
| Stage II | The tumor extends to the cervix. |
| Stage IIA | Cervical extension is limited to the |
| | endocervical glands. |
| Stage IIB | Tumor invades the cervical stroma. |
| Stage III | Regional tumor spread. |
| Stage IIIA | The tumor invades the uterine serosa, or |
| | adnexa (ovary), or cells in the peritoneum |
| | show signs of cancer. |
| Stage IIIB | Vaginal metastases are present. |
| Stage IIIC | Tumor spread to lymph nodes near the uterus. |
| Stage IV | Bulky pelvic disease or distant spread. |
| Stage IVA | Tumor spread to the bladder or rectum. |
| Stage IVB | Distant metastases are present. |

TABLE 1B
FIGO Clinical Staging System for Uterine Cancer

| | |
|---|---|
| Stage 1 | Tumor limited to the uterine body. |
| Stage 1A | Uterine cavity measures 8 cm or less. |
| Stage 1B | Uterine cavity measures greater than 8 cm. |
| Stage 2 | Tumor extends to the uterine cervix. |
| Stage 3 | Tumor spread to the adjacent pelvic structures. |
| Stage 4 | Bulky pelvic disease or distant spread. |
| Stage 4A | The tumor invades the mucosa of the bladder |
| | or rectum. |
| Stage 4B | Distant metastasis is present. |

TABLE 1C
FIGO Stages For Ovarian Cancer

| | |
|---|---|
| Stage I | Cancer is limited to the ovaries. |
| Stage IA | Limited to one ovary and the outer ovarian capsule |
| | is not ruptured. There is no tumor on the external |
| | surface. No ascites fluid and washings are negative |
| | for malignant cells |
| Stage IB | Cancer is present in both ovaries. The outer |
| | capsule is intact and there is no tumor on the |
| | external surface. No ascites fluid and washings are |
| | negative for malignant cells. |
| Stage IC | Cancer is either at Stage IA or IB and the capsule |
| | is ruptured or there is a tumor on the ovarian |
| | surface or malignant cells are present in ascites or |
| | washings. |
| Stage II | Cancer involves at least one ovary with spread to |
| | other pelvic organs or surfaces. |
| Stage IIA | Cancer has extended, implanted, or spread cells |
| | onto the uterus and/or fallopian tube. There is no |
| | ascites fluid and the washings are negative for |
| | malignant cells. |
| Stage IIB | Cancer has extended, implanted, or spread cells |
| | onto other pelvic tissues. There is no ascites fluid |
| | and the washings are negative for malignant cells. |
| Stage III | Cancer has spread outside of the pelvis to the |
| | abdominal area, including metastases to the liver |
| | surface. |
| Stage IIIA | Tumor is grossly confined to the pelvis but with |
| | microscopic peritoneal metastases beyond the |
| | pelvis to abdominal peritoneal surfaces or the |
| | omentum. |
| Stage IIIB | Tumor is grossly confined to the pelvis but with |
| | microscopic peritoneal metastases beyond the |
| | pelvis to abdominal peritoneal surfaces or the |
| | omentum. Microscopic metastases are less than 2 |
| | cm in size. |
| Stage IIIC | Tumor is grossly confined to the pelvis but with |
| | microscopic peritoneal metastases beyond the |
| | pelvis to abdominal peritoneal surfaces or the |
| | omentum. Microscopic metastases are greater than |
| | 2 cm in size or there are lymph node metastases to |
| | inguinal, pelvic, or paraaoric areas. |
| Stage IV | Metastases are spread to the liver or outside the |
| | peritoneal cavity to areas such as the chest or brain. |

Tumors are also graded according to histopathology and provided a histopathologic grade. Accordingly, the histopathologic grade is a qualitative assessment of the differentiation of the tumor expressed as the extent to which a tumor resembles normal tissue present at the site. Grade is expressed numerically from most differentiated (Grade 1) to least differentiated (Grade 4). Exemplary histopathologic grades include, but are not limited to, GX=histopathological grade cannot be determined, G1=well-differentiated, G2=moderately differentiated, G3=poorly differentiated, and G4=undifferentiated.

Histopathologic type is a qualitative pathologic assessment wherein the tumor is characterized or typed according to the normal tissue type of cell type it most closely resembles. In general, the World Health Organization International Histologic Classification of Tumors is for histopathologic typing (WHO International Classification of Diseases for Oncology ICD-O (3rd edition), World Health Organization, Geneva, 2000).

Alternatively, or in addition, severity is meant to describe the tumor grade by art-recognized methods (see, National Cancer Institute, www.cancer.gov). Tumor grade is a system used to classify cancer cells in terms of how abnormal the cells look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized.

### Endometrial/Uterine Cancer

Most endometrial cancers are adenocarcinomas, so named because these cancers originate from the single layer of epithelial cells that line the endometrium and form the endometrial glands. There are multiple subtypes of endometrial carcinoma, including, but not limited to the common endometrioid type, and the more aggressive papillary serous carcinoma and clear cell endometrial carcinomas.

Subtypes are optionally categorized as Type I or Type II endometrial carcinomas based on low- or high-grade status. Type I endometrial carcinomas are often minimally invasive into the underlying uterine wall, include the low-grade endometrioid type, and typically provide a good prognosis. In sharp contrast, Type II endometrial carcinomas provide a poorer prognosis. Exemplary Type II cancers include, but are not limited to, high-grade endometrioid cancer, uterine papillary serous carcinoma, and uterine clear cell carcinoma.

Importantly, these subtypes are readily distinguishable by simple microscopic evaluation. For instance, low-grade endometrioid carcinoma cells resemble cells of the normal endometrium. High-grade endometrioid carcinoma cells are poorly differentiated compared to low grade endometrioid carcinoma cells. In contrast, uterine papillary serous carcinoma tumors are characterized by nipple-shaped structures (papillae) with fibrovascular cores, marked nuclear atypia (irregularities in the nuclear membrane, enlarged nuclear size), psammoma bodies, and cilia. Moreover, uterine clear cell carcinoma is characterized as having large clear cells with enlarged, angulated nuclei and tumors with distinct margins, papillary, glandular, or sheet-like architectural formations.

Endometrial stromal sarcomas are uncommon subtype of endometrial cancers that originate in the non-glandular connective tissue of the endometrium. Uterine carcinosarcoma is a rare uterine cancer containing cancerous cells of both glandular and sarcomatous appearance.

Cancer of the uterine corpus is the most common gynecologic malignancy, and eighth leading cause of female death. 94% of uterine cancers are carcinomas and uterine papillary serous carcinomas (UPSCs) account for 10% of those cases. In contrast to their endometrioid counterparts, these tumors occur in older (median age 65-70), non-obese and parous patients. UPSCs are highly aggressive, commonly present at an advanced stage and have a 5-year overall survival of 42%.

Uterine papillary serous tumors have complex papillary architecture, which resembles papillary serous carcinoma of the ovary; psammoma bodies are present in 60 percent of cases. Several biologic markers correlate with biology and prognosis of UPSCs. Mutation and consequent overexpression of p53, overexpression of MIB-1/Ki-67, abnormal DNA ploidy, and increased S-phase fraction, DNA methylation, or expression of p21 are unfavorable markers. Estrogen and progesterone receptor positivity is a good prognostic marker.

### Ovarian Cancer

Ovarian cancer is the second most common gynecologic malignancy and the leading cause of mortality from gynecologic cancer. Approximately 22,000 women in the United States are diagnosed with ovarian cancer annually, and an estimated 15,000 women die of their disease. Overall survival, the need for adjuvant therapy and the risk of recurrence in epithelial ovarian carcinomas (EOC) is greatly dependent on the stage of disease at presentation (see, Table 1C). Because EOC presents vague initial symptoms and often precludes early detection, metastatic disease is most frequently present at diagnosis. When ovarian carcinoma is believed to be a metastatic tumor, the uterus is a common site for such metastatic disease.

EOCs arise from neoplastic transformation of coelomic epithelium and adjacent ovarian stroma. Papillary serous histology account for 75% of ovarian cancers. Gene expression profiling of ovarian carcinoma has been extensively explored. Multiple potential diagnostic markers have been identified including osteopontin, YKL-40, CA 15-3, and composite markers (Kim, J H, et al. JAMA 2002; 287:1671; Dupont, J, Tanwar, M K, Thaler, H T, et al. J Clin Oncol 2004; 22:3330; and McIntosh, M W et al. Gynecol Oncol 2004; 95:9.)

### Concurrent Endometrial and Ovarian Cancers

Risk factors for synchronous endometrial and ovarian cancers include younger age, obesity, premenopausal status, and nulliparity, which suggest a hormonal field effect. If the histology of both sites is dissimilar, the diagnosis of simultaneous malignancies is uncomplicated. However, when the histology of both sites is papillary serous, correct diagnosis is exceedingly challenging for the clinicians and pathologists. Such tumors could present one of the three conditions: (a) primary endometrial cancer with ovarian metastasis, (b) primary ovarian cancer with endometrial metastasis, or (c) true synchronous primary endometrial and ovarian cancers. This pathologic differentiation is critical because it influences cancer staging, adjuvant therapy, and information about prognosis. Previous studies pointed out that the prognosis of synchronous primary cancers of the endometrium and ovary, in low grade and stage, is favorable, and differs greatly from much higher stage of metastatic disease of a single organ.

Multiple pathologic criteria, including molecular analysis developments, have been proposed to distinguish synchronous primary cancers from metastatic lesions. Ulbright et al. proposed pathologic criteria for differentiation in 1985, including either a multinodular ovarian pattern (major criterion) or two or more of the following minor criteria: small (less than 5 cm) ovary(ies), bilateral ovarian involvement, deep myometrial invasion, vascular invasion, and tubal lumen involvement (Ulbright T. M and Roth L. M. Hum Pathol 1985; 16: 28-34). Scully et al. further developed the pathologic criteria (Scully, R. et al. Atlas of Tumor Pathology 1998; 23: Table 5-1 to 5-3). Several methods of molecular analysis had been developed to aid in differentiating synchronous primary tumors from metastatic disease, such as DNA flow cytometry, loss of heterozygosity on chromosome, X-chromosome inactivation, PTEN/MMAC1, beta-catenin, and microsatellite instability (Soliman, P. T. et al. Gynecol Oncol 2004; 94:456-62; Lu, J. et al. Nature 2005; 435: 834-8). Currently, there is no consensus about the most appropriate discriminating method and diagnosis depends mainly on morphologic pathologic criteria.

Previous studies from our group found that miRNA signatures of endometrial cancers can differentiate subtypes of endometrial cancer, including UPSC. The miRNA signature of EOCs has been reported as well. Importantly, in these previous studies, these subtypes of endometrial cancer were distinguishable by histological means, as well as miRNA signatures. In other words, cellular analyses of biopsy samples obtained from patients could classify which of these subtypes were present in the patient because the different subtypes have different cellular morphology. Furthermore, in these previous studies, one would have expected that cancer cells having different cellular morphologies would have different gene expression patterns, and consequently, distinct, miRNA signatures that could validate that histological determination of cancer subtype.

In stark contrast to this previous work, the present invention provides a method of identifying tumors of the same subtype but from different origins (ovarian and uterine). Using histological analyses of tumor subtype, uterine and ovarian serous papillary tumors otherwise appear identical.

Moreover, miRNA expression patterns can identify the tissue of origin of metastatic cancers. MiRNAs that are differentially expressed in each primary cancer tissue retain their miRNA “signatures” even after that primary tumor tissue has metastasized to another location in the body.

The invention provides a papillary serous miRNA signatures and a superior method of differentiating seemingly identical tumors by applying the miRNA signature and/or expression levels to these tumors. Moreover the ability of the claimed miRNA signature to differentiate morphologically- and histologically-identical tumors is unexpected. Cell morphology and protein expression are determined by gene expression. Thus, if the cells look identical, and express the similar genes, one would expect the cells to regulate gene expression in a similar way. However, miRNA expression levels are statistically significantly different for the miRNAs that comprise the papillary serous miRNA signature described in Example 2 and Table 4. Furthermore, the determination of tumor origin using this miRNA signature is “binary.” For instance, an unknown tumor either displays an increase in expression of 10-11 miRNAs of the papillary serous miRNA signature, indicating a uterine origin, or the unknown tumor displays an absence of increased expression in 10-11 miRNAs of the miRNA signature, indicating an ovarian origin. The unknown tumor does not display an “ambiguous” result. For instance, the unknown tumor will display a statistically significantly changed expression, e.g. significantly increased or decreased expression, of 5 or 6 miRNAs of the papillary serous miRNA signature.

The binary quality of the papillary serous miRNA signature described in Example 2 and Table 4 is the result of two steps, one normalization and one threshold step, in the analysis of miRNA expression in uterine versus ovarian papillary serous tumor samples. The first decision is which RNA control to use in the miRNA microarray analysis, to which the expression levels of a miRNA of interest are normalized prior to comparing expression levels of identified miRNAs across tissue types. Optimal normalization control RNAs are highly and invariably expressed in most tissue types (and particularly among tissue types of interest), belong to the group of non-coding RNAs ranging in size from between 20 and 500 nucleotides, but preferably between 45 and 200 nucleotides, and comprise at least one of the following forms, including, but not limited to, transfer RNA (tRNA), small nuclear RNA (snRNA) and small nucleolar RNA (snoRNA).The second decision is the threshold level of statistical significance that is required to separate those miRNAs that predictably identify tumor samples with minimal chance of error from uninformative miRNAs. Based upon these decisions, a miRNA signature is determined that provides a binary choice between two cancer origins, e.g. uterine and ovarian tissue origins.

The papillary serous miRNA signature described in Example 4 and Table 5 also provides a superior method of differentiating seemingly identical tumors by applying the miRNA signature to these tumors. MiRNA expression levels are statistically significantly different between uterine and ovarian cells for the miRNAs that comprise this papillary serous miRNA signature. The miRNAs of this signature were identified following an optimization of the normalization step which allows for validation of a greater number of miRNAs by eliminating the step of normalizing the expression levels within each of 8 pools of miRNA reactions (containing 30-40 miRNAs each) prior to comparing the values between pools. The preferred method of data normalization is a single reaction that contains every miRNA being evaluated, and therefore, contains only a single normalization step. The singular reaction decreases variability between reaction pools and the single normalization preserves the “signal to noise” ratio of the data, allowing statistically significant differences to emerge above the background. Moreover, the second papillary serous miRNA signature was determined using new microarray plates (Applied Biosystems 7900 Low Density Array Panel plates), which contain the most current primers drawn to the most updated miRNA sequences available in the miRBase Database (publicly available at www.mirbase.org).

It is expected that, by varying the first and second thresholds above, or by applying the methods herein, one or more miRNA signatures are developed that further differentiate papillary serous tumors arising from tissue of the fallopian tubes or the peritoneum from tumors arising in the uterus and ovary. The fallopian tubes and peritoneum are two additional tissues from which malignant tumor cells metastasize to the uterus and ovary. As such, when concurrent cancers occur in the uterus, ovary, fallopian tube, and/or peritoneum, at least one miRNA signature is applied to tumors from each of the above tissues to distinguish uterine and ovarian origins, uterine and fallopian tube origins, uterine and peritoneum origins, ovary and fallopian tube origins, and fallopian tube and peritoneum origins. Thus, miRNA signatures are applied to tumors within the fallopian tubes and peritoneum, to determine the tissue origin, presence of synchronous primary, or metastatic disease, as described herein for uterine and ovarian papillary serous carcinoma.

### MicroRNA Signatures

MiRNAs are a broad class of small non-protein-coding RNA molecules of approximately 22 nucleotides in length that function in posttranscriptional gene regulation by pairing to the mRNA of protein-coding genes. Recently, it has been shown that miRNAs play roles at human cancer loci with evidence that they regulate proteins known to be critical in survival pathways (Esquela-Kerscher, A. & Slack, and F. J. Oncomirs—microRNAs with a role in cancer. Nat Rev Cancer 2006. 6, 259-69; Ambros, V. Cell 2001. 107, 823-6; Slack, F. J. and Weidhaas, J. B. Future Oncol 2006. 2, 73-82). Because miRNAs control many downstream targets, it is possible for them to act as novel targets for the treatment in cancer.

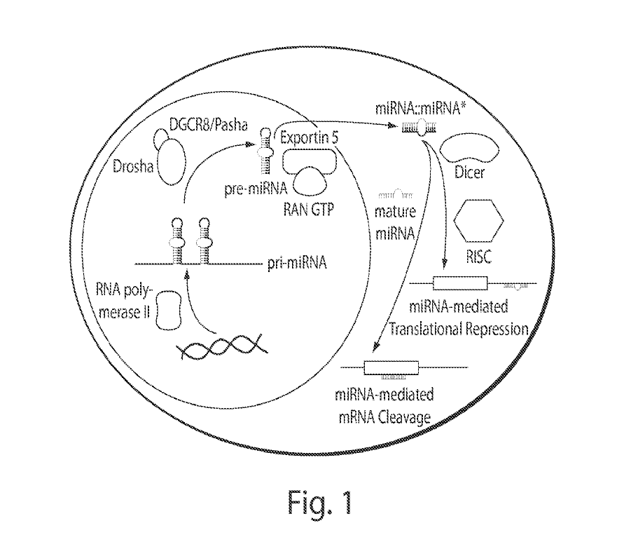

The basic synthesis and maturation of miRNAs can be visualized in FIG. 1 (Esquela-Kerscher, A. and Slack, F. J. Nat Rev Cancer 2006. 6, 259-69). In brief, miRNAs are transcribed from miRNA genes by RNA Polymerase II in the nucleus to form long primary RNAs (pri-miRNA) transcripts, which are capped and polyadenylated (Esquela-Kerscher, A. and Slack, F. J. Nat Rev Cancer 2006. 6, 259-69; Lee, Y. et al. Embo J 2002. 21, 4663-70). These pri-miRNAs can be several kilobases long, and are processed in the nucleus by the RNAaseIII enzyme Drosha and its cofactor, Pasha, to release the approximately 70-nucleotide stem-loop structured miRNA precursor (pre-miRNA). Pre-miRNAs are exported from the nucleus to the cytoplasm by exportin 5 in a Ran-guanosine triphosphate (GTP)-dependent manner, where they are then processed by Dicer, an RNase III enzyme. This causes the release of an approximately 22-base nucleotide, double-stranded, miRNA:miRNA duplex that is incorporated into a RNA-induced silencing complex (miRISC). At this point the complex is now capable of regulating its target genes.

FIG. 1 depicts how gene expression regulation can occur in one of two ways that depends on the degree of complimentarity between the miRNA and its target. MiRNAs that bind to mRNA targets with imperfect complimentarity block target gene expression at the level of protein translation. Complimentary sites for miRNAs using this mechanism are generally found in the 3′ UTR of the target mRNA genes. MiRNAs that bind to their mRNA targets with perfect complimentarity induce target-mRNA cleavage. MiRNAs using this mechanism bind to miRNA complimentary sites that are generally found in the coding sequence or open reading frame (ORF) of the mRNA target.

In mammals, miRNAs are gene regulators that are found at abnormal levels in virtually all cancer subtypes studied. Proper miRNA binding to their target genes is critical for regulating the mRNA level and protein expression.

The invention provides method of assessing the expression levels of, for instance, the miRNAs provided in Table 2. The human miRNAs on this list are nonlimiting examples of miRNAs expressed in cancerous cells (miRNAs beginning with the letters “hsa”), as well as RNAs, which are useful as controls for real-time polymerase chain reaction (RT-PCR) (miRNAs not beginning with the letters “hsa”), as described above. The miRNAs provided in Table 2 are not meant to be an exhaustive list of all known human miRNAs or all possible miRNAs that may be included in the signatures or methods described herein. Rather, the miRNAs provided in Table 2 are illustrative of human miRNAs that can be considered for use in a signature or method of the invention.

According to the methods described in Example 1, the human miRNA sequences below may be isolated, cloned, sorted, amplified, detected or otherwise manipulated as either RNA (shown in Table 2), DNA, complementary DNA (cDNA), synthetic RNA or DNA, or synthetic oligonucleotides. DNA, complementary DNA (cDNA), synthetic RNA or DNA, or synthetic oligonucleotide sequences corresponding to the miRNA sequences provided in Table 2 may be identical to the sequences provided in Table 2, or may contain substitutions of the specified uracil (U) nucleobase for a thymine (T) nucleobase. Synthetic RNA, DNA, and oligonucleotides are generated in vitro, by methods known in the art, including, but not limited to, solid phase synthesis in silica and commercial grade synthesizers such as, Applied Biosystems 394 or 3900 Synthesizers that use beta-cyanoethyl chemistry.

To generate a miRNA signature to distinguish between one or more cancer subtypes, the relative expression levels of all miRNAs present in the cancer cells of each subtype are determined with respect to a control RNA of known abundance. Alternatively, or in addition, the absolute expression levels of each miRNA are determined through a calculation that compares the relative levels to the known control level. Moreover, relative expression levels of all miRNAs present in the cancer cells of each subtype are normalized to a highly- and invariably-expressed control RNA. The term “invariably-expressed RNA” is meant to describe an RNA, of which the expression level and pattern is similar in each of the tissues from which the compared cancer subtypes arise. Expression patterns are both spatial and temporal. The normalized miRNA expression levels are further compared between one or more cancer subtypes. MiRNAs that are expressed in one or more of the cancer subtypes are included in a cancer subtype-specific miRNA signature, exclusive expression in one subtype over another is not required. However, when a miRNA of a miRNA signature is expressed in more than one cancer subtype, the expression level of that miRNA must be statistically significantly different, as determined by a p-value of 0.1 or less. Preferably, a p-value is 0.05 or less, or even more preferred are p-values of 0.01 or less.

TABLE 2
Experimental and Control Human miRNAs

| SEQ ID NO: | Mature Sequence | miRBase ™ ID |
|---|---|---|
| 14 | UGAGGUAGUAGGUUGUAUAGUU | hsa-let-7a |
| 15 | UGAGGUAGUAGGUUGUGUGGUU | hsa-let-7b |
| 16 | UGAGGUAGUAGGUUGUAUAGUU | hsa-let-7c |
| 17 | AGAGGUAGUAGGUUGCAUAGU | hsa-let-7d |
| 18 | UGAGGUAGGAGGUUGUAUAGU | hsa-let-7e |
| 19 | UGAGGUAGUAGAUUGUAUAGUU | hsa-let-7f |
| 20 | UGAGGUAGUAGUUUGUACAGU | hsa-let-7g |
| 21 | UGAGGUAGUAGUUUGUGCUGU | hsa-let-7i |
| 22 | UGGAAUGUAAAGAAGUAUGUA | hsa-miR-1 |
| 23 | UGGAAGACUAGUGAUUUUGUUG | hsa-miR-7 |
| 24 | UACCCUGUAGAUCCGAAUUUGUG | hsa-miR-10a |
| 25 | UACCCUGUAGAACCGAAUUUGU | hsa-miR-10b |
| 26 | UAGCAGCACAUAAUGGUUUGUG | hsa-miR-15a |
| 27 | UAGCAGCACAUCAUGGUUUACA | hsa-miR-15b |
| 28 | UAGCAGCACGUAAAUAUUGGCG | hsa-miR-16 |
| 29 | ACUGCAGUGAAGGCACUUGU | hsa-miR-17-3p |
| 30 | CAAAGUGCUUACAGUGCAGGUAGU | hsa-miR-17-5p |
| 31 | UAAGGUGCAUCUAGUGCAGAUA | hsa-miR-18a |
| 3 | UGUGCAAAUCUAUGCAAAACUGA | hsa-miR-19a |
| 8 | UGUGCAAAUCCAUGCAAAACUGA | hsa-miR-19b |
| 32 | UAGCUUAUCAGACUGAUGUUGA | hsa-miR-21 |
| 33 | AAGCUGCCAGUUGAAGAACUGU | hsa-miR-22 |
| 34 | AUCACAUUGCCAGGGAUUUCC | hsa-miR-23a |
| 35 | AUCACAUUGCCAGGGAUUACC | hsa-miR-23b |
| 6 | UGGCUCAGUUCAGCAGGAACAG | hsa-miR-24 |
| 36 | CAUUGCACUUGUCUCGGUCUGA | hsa-miR-25 |
| 37 | UUCAAGUAAUCCAGGAUAGGC | hsa-miR-26a |
| 38 | UUCAAGUAAUCCAGGAUAGGCU | hsa-miR-26a |
| 39 | UUCAAGUAAUUCAGGAUAGGUU | hsa-miR-26b |
| 40 | UUCAAGUAAULTCAGGAUAGGU | hsa-miR-26b |
| 41 | UUCACAGUGGCUAAGUUCCGC | hsa-miR-27a |
| 42 | UUCACAGUGGCUAAGLTOCUGC | hsa-miR-27b |
| 43 | AAGGAGCUCACAGUCUAUUGAG | hsa-miR-28 |
| 44 | UAGCACCAUCUGAAAUCGGUU | hsa-miR-29a |
| 45 | UAGCACCAUUUGAAAUCAGUGUU | hsa-miR-29b |
| 46 | UAGCACCAUUUGAAAUCGGU | hsa-miR-29c |
| 47 | CUUUCAGUCGGAUGUUUGCAGC | hsa-miR-30a-3p |
| 48 | UGUAAACAUCCUCGACUGGAAC | hsa-miR-30a-5p |
| 49 | UGUAAACAUCCUACACUCUCAGC | hsa-miR-30c |
| 50 | UGUAAACAUCCCCGACUGGAAG | hsa-miR-30d |
| 51 | UGUAAACAUCCUUGACUGGA | hsa-miR-30e-5p |
| 52 | CUUUCAGUCGGAUGUUUACAGC | hsa-miR-30e-3p |
| 53 | UAUUGCACAUUACUAAGUUGC | hsa-miR-32 |
| 54 | GUGCAUUGUAGUUGCAUUG | hsa-miR-33 |
| 55 | UGGCAGUGUCUUAGCUGGUUGUU | hsa-miR-34a |
| 56 | UGGCAGUGUCUUAGCUGGUUGU | hsa-miR-34a |
| 57 | UAGGCAGUGUCAUUAGCUGAUUG | hsa-miR-34b |
| 58 | AGGCAGUGUAGUUAGCUGAUUGC | hsa-miR-34c |
| 59 | UAUUGCACLTUGUCCCGGCCUG | hsa-miR-92 |
| 60 | UAUUGCACUUGUCCCGOCCUGU | hsa-miR-92a |
| 61 | AAAGUGCUGUUCGUGCAGGUAG | hsa-miR-93 |
| 62 | UUCAACGGGUAUUUAUUGAGCA | hsa-miR-95 |
| 63 | UUUGGCACUAGCACAUUULTUGC | hsa-miR-96 |
| 64 | AACCCGUAGAUCCGAUCUUGUG | hsa-miR-99a |
| 65 | CACCCGUAGAACCGACCUUGCG | hsa-miR-99b |
| 66 | AACCCGUAGAUCCGAACUUGUG | hsa-miR-100 |
| 67 | UACAGUACUGUGAUAACUGAAG | hsa-miR-101 |
| 68 | AGCAGCAUUGUACAGGGCUAUGA | hsa-miR-103 |
| 69 | UCAAAUGCUCAGACUCCUGU | hsa-miR-105 |
| 70 | UAAAGUGCUGACAGUGCAGAU | hsa-miR-106b |
| 71 | AGCAGCAUUGUACAGGGCUAUCA | hsa-miR-107 |
| 72 | UGGAGUGUGACAAUGGUGUUUGU | hsa-miR-122a |
| 73 | UUAAGGCACGCGGUGAAUGCCA | hsa-miR-124a |
| 74 | UCCCUGAGACCCUUUAACCUGUG | hsa-miR-125a |
| 75 | UCCCUGAGACCCUAACUUGUGA | hsa-miR-125b |
| 76 | UCGUACCGUGAGUAAUAAUGC | hsa-miR-126 |
| 77 | CAUUAUUACUUUUGGUACGCG | hsa-miR-126* |
| 78 | UCGGAUCCGUCUGAGCUUGGCU | hsa-miR-127 |
| 79 | UCACAGUGAACCGGUCUCUUUU | hsa-miR-128a |
| 80 | CAGUGCAAUGUUAAAAGGGCAU | hsa-miR-130a |
| 81 | CAGUGCAAUGAUGAAAGGGCAU | hsa-miR-130b |
| 82 | UAACAGUCUACAGCCAUGGUCG | hsa-miR-132 |
| 83 | UUGGUCCCCUUCAACCAGCUGU | hsa-miR-133a |
| 84 | UGUGACUGGUUGACCAGAGGG | hsa-miR-134 |
| 85 | UAUGGCUUUUUAUUCCUAUGUGA | hsa-miR-135a |
| 86 | UAUGGCUUUUCAUUCCUAUGUG | hsa-miR-135b |
| 87 | AGUGGUUTJUACCCUAUGGUAG | hsa-miR-140 |
| 1 | UAACACUGUCUGGUAAAGAUGG | hsa-miR-141 |
| 5 | UGUAGUGUUUCCUACUUUAUGGA | hsa-miR-142-3p |
| 644 | CAUAAAGUAGAAAGCACUAC | hsa-miR-142-5p |
| 88 | UGAGAUGAAGCACUGUAGCUCA | hsa-miR-143 |
| 89 | GUCCAGUUUUCCCAGGAAUCCCUU | hsa-miR-145 |
| 90 | UGAGAACUGAAUUCCAUGGGUU | hsa-miR-146a |
| 91 | GUGUGUGGAAAUGCUUCUGC | hsa-miR-147 |
| 92 | UCAGUGCACUACAGAACUUUGU | hsa-miR-148a |
| 93 | UCAGUGCAUCACAGAACUUUGU | hsa-miR-148b |
| 94 | UCUGGCUCCGUGUCUUCACUCC | hsa-miR-149 |
| 95 | UCUCCCAACCCUUGUACCAGUG | hsa-miR-150 |
| 96 | UCAGUGCAUGACAGAACUUGGG | hsa-miR-152 |
| 97 | UCAGUGCAUGACAGAACUUGG | hsa-miR-152 |
| 98 | UUGCAUAGUCACAAAAGUGA | hsa-miR-153 |
| 100 | UAGGUUAUCCGUGUUGCCUUCG | hsa-miR-154 |
| 101 | AAUCAUACACGGUUGACCUAUU | hsa-miR-154* |
| 831 | UUAAUGCUAAUCGUGAUAGGGG | hsa-miR-155 |
| 103 | AACAUUCAACGCUGUCGGUGAGU | hsa-miR-181a |
| 104 | AACAUUCAACCUGUCGGUGAGU | hsa-miR-181c |
| 105 | UGGUUCUAGACUUGCCAACUA | hsa-miR-182* |
| 106 | UAUGGCACUGGUAGAAUUCACUG | hsa-miR-183 |
| 107 | UGGACGGAGAACUGAUAAGGGU | hsa-miR-184 |
| 108 | CAAAGAAUUCUCCUUUUGGGCUU | hsa-miR-186 |
| 109 | UCGUGUCUUGUGUUGCAGCCG | hsa-miR-187 |
| 110 | GUGCCUACUGAGCUGAUAUCAGU | hsa-miR-189 |
| 111 | UGAUAUGUUUGAUAUAUUAGGU | hsa-miR-190 |
| 112 | CAACGGAAUCCCAAAAGCAGCU | hsa-miR-191 |
| 113 | CUGACCUAUGAAUUGACAGCC | hsa-miR-192 |
| 114 | AACUGGCCUACAAAGUCCCAG | hsa-miR-193a |
| 115 | UGUAACAGCAACUCCAUGUGGA | hsa-miR-194 |
| 116 | UAGCAGCACAGAAAUAUUGGC | hsa-miR-195 |
| 117 | UAGGUAGUUUCAUGUUGUUGG | hsa-miR-196a |
| 118 | UAGGUAGUUUCCUGUUGUUGG | hsa-miR-196b |
| 119 | UUCACCACCUUCUCCACCCAGC | hsa-miR-197 |
| 120 | CCCAGUGUUCAGACUACCUGUUC | hsa-miR-199a |
| 121 | UACAGUAGUCUGCACAUUGGUU | hsa-miR-199a* |
| 122 | CCCAGUGUUUAGACUAUCUGUUC | hsa-miR-199b |
| 123 | UAACACUGUCUGGUAACGAUGU | hsa-miR-200a |
| 124 | UAAUACUGCCGGGUAAUGAUGG | hsa-miR-200c |
| 125 | GUGAAAUGUUUAGGACCACUAG | hsa-miR-203 |
| 126 | UUCCCUUUGUCAUCCUAUGCCU | hsa-miR-204 |
| 127 | UCCUUCAUTJCCACCGGAGUCUG | hsa-miR-205 |
| 128 | UGGAAUGUAAGGAAGUGUGUGG | hsa-miR-206 |
| 129 | AUAAGACGAGCAAAAAGCUUGU | hsa-miR-208 |
| 130 | CUGUGCGUGUGACAGCGGCUGA | hsa-miR-210 |
| 131 | UUCCCUUUGUCAUCCUUCGCCU | hsa-miR-211 |
| 132 | UAACAGUCUCCAGUCACGGCC | hsa-miR-212 |
| 133 | ACCAUCGACCGUUGAUUGUACC | hsa-miR-213 |
| 134 | ACAGCAGGCACAGACAGGCAG | hsa-miR-214 |
| 135 | AUGACCUAUGAAUUGACAGAC | hsa-miR-215 |
| 136 | UAAUCUCAGCUGGCAACUGUG | hsa-miR-216 |
| 137 | UACUGCAUCAGGAACUGAUUGGAU | hsa-miR-217 |
| 138 | UUGUGCUUGAUCUAACCAUGU | hsa-miR-218 |
| 139 | UGAUUGUCCAAACGCAAUUCU | hsa-miR-219 |
| 140 | CCACACCGUAUCUGACACUUU | hsa-miR-220 |
| 141 | AGCUACAUUGUCUGCUGGGUUUC | hsa-miR-221 |
| 142 | AGCUACAUCUGGCUACUGGGUCUC | hsa-miR-222 |
| 688 | UGUCAGUUUGUCAAAUACCCC | hsa-miR-223 |
| 143 | AGGGCCCCCCCUCAAUCCUGU | hsa-miR-296 |
| 144 | CAGUGCAAUAGUA UUGUCAAAGC | hsa-miR-301 |
| 145 | UAAGUGCUUCCAUGUUUUGGUGA | hsa-miR-302a |
| 146 | UAAACGUGGAUGUACUUGCUUU | hsa-miR-302a* |
| 147 | UAAGUGCUUCCAUGUUUUAGUAG | hsa-miR-302b |
| 148 | ACUUUAACAUGGAAGUGCUUUCU | hsa-miR-302b |
| 149 | UAAGUGCUUCCAUGUUUCAGUGG | hsa-miR-302c |
| 150 | UUTJAACAUGGGGGUACCUGCUG | hsa-miR-302c* |
| 151 | UAAGUGCUUCCAUGUUUGAGUGU | hsa-miR-302d |
| 152 | AAAAGCUGGGUUGAGAGGGCGAA | hsa-miR-320 |
| 153 | GCACAUUACACGGUCGACCUCU | hsa-miR-323 |
| 154 | CGCAUCCCCUAGGGCAUUGGUGU | hsa-miR-324-5p |
| 155 | CCUAGUAGGUGUCCAGUAAGUGU | hsa-miR-325 |
| 156 | CCUCUGGGCCCUUCCUCCAG | hsa-miR-326 |
| 157 | CUGGCCCUCUCUGCCCUUCCGU | hsa-miR-328 |
| 158 | GCAAAGCACACGGCCUGCAGAGA | hsa-miR-330 |
| 159 | GCCCCUGGGCCUAUCCUAGAA | hsa-miR-331 |
| 160 | UCAAGAGCAAUAACGAAAAAUGU | hsa-miR-335 |
| 161 | UCCAGCUCCUAUAUGAUGCCUUU | hsa-miR-337 |
| 162 | UCCAGCAUCAGUGAUUUUGUUGA | hsa-miR-338 |
| 163 | UCCCUGUCCUCCAGGAGCUCA | hsa-miR-339 |
| 164 | UCCGUCUCAGUUACUUUAUAGCC | hsa-miR-340 |
| 165 | UCUCACACAGAAAUCGCACCCGUC | hsa-miR-342 |
| 166 | UGCUGACUCCUAGUCCAGGGC | hsa-miR-345 |
| 167 | UGUCUGCCCGCAUGCCUGCCUCU | hsa-miR-346 |
| 168 | UUAUCAGAAUCUCCAGGGGUAC | hsa-miR-361 |
| 169 | AAUUGCACUUUAGCAAUGGUGA | hsa-miR-367 |
| 170 | ACAUAGAGGAAAUUCCACGUUU | hsa-miR-368 |
| 171 | AAUAAUACAUGGUUGAUCUUU | hsa-miR-369-3p |
| 172 | GCCUGCUGGGGUGGAACCUGG | hsa-miR-370 |
| 173 | GUGCCGCCAUCUUUUGAGUGU | hsa-miR-371 |
| 174 | AAAGUGCUGCGACAUUUGAGCGU | hsa-miR-372 |
| 175 | GAAGUGCUUCGAUUUUGGGGUGU | hsa-miR-373 |
| 176 | ACUCAAAAUGGGGGCGCUUUCC | hsa-miR-373* |
| 177 | UUAUAAUACAACCUGAUAAGUG | hsa-miR-374 |
| 178 | UUUGUUCGUUCGGCUCGCGUGA | hsa-miR-375 |
| 179 | AUCAUAGAGGAAAAUCCACGU | hsa-miR-376a |
| 180 | AUCACACAAAGGCAACUUUUGU | hsa-miR-377 |
| 181 | CUCCUGACUCCAGGUCCUGUGU | hsa-miR-378 |
| 182 | UGGUAGACUAUGGAACGUA | hsa-miR-379 |
| 183 | UAUGUAAUAUGGUCCACAUCUU | hsa-miR-380-3p |
| 184 | UGGUUGACCAUAGAACAUGCGC | hsa-miR-380-5p |
| 185 | UAUACAAGGGCAAGCUCUCUGU | hsa-miR-381 |
| 186 | GAAGUUGUUCGUGGUGGAUUCG | hsa-miR-382 |
| 187 | AGAUCAGAAGGUGAUUGUGGCU | hsa-miR-383 |
| 188 | AUUCCUAGAAAUUGUUCAUA | hsa-miR-384 |
| 189 | CUGGACUUGGAGUCAGAAGGCC | hsa-miR-422b |
| 190 | AGCUCGGUCUGAGGCCCCUCAG | hsa-miR-423 |
| 191 | UGAGGUAGUAAGUUGUAUUGUU | hsa-miR-98 |
| 192 | AAAAGUGCUUACAGUGCAGGUAGC | hsa-miR-106a |
| 193 | CCACUGCCCCAGGUGCUGCUGG | hsa-miR-324-3p |
| 194 | UAAAGUGCUUAUAGUGCAGGUAG | hsa-miR-20a |
| 195 | GGUCCAGAGGGGAGAUAGG | hsa-miR-198 |
| 196 | UCUUUGGUUAUCUAGCUGUAUGA | hsa-miR-9 |
| 197 | UAAAGCUAGAUAACCGAAAGU | hsa-miR-9* |
| 198 | UAGCACCAUUUGAAAUCGGUUA | hsa-miR-29e |
| 199 | UCACAGUGAACCGGUCUCUUUC | hsa-miR-128b |
| 200 | CUUUUUGCGGUCUGGGCUUGC | hsa-miR-129 |
| 201 | UUGGUCCCCUUCAACCAGCUA | hsa-miR-133b |
| 202 | ACUCCAUUUGUUUUGALTGAUGGA | hsa-miR-136 |
| 203 | UAUUGCUUAAGAAUACGCGUAG | hsa-miR-137 |
| 204 | AGCUGGUGUUGUGAAUC | hsa-miR-138 |
| 205 | ACUAGACUGAAGCUCCUUGAGG | hsa-miR-151 |
| 206 | UUUGGCAALIGGUAGAACUCACA | hsa-miR-182 |
| 207 | UGGAGAGAAAGGCAGUUC | hsa-miR-185 |
| 208 | CAAGUCACUAGUGGUUCCGUUUA | hsa-miR-224 |
| 209 | UGGUUUACCGUCCCACAUACAU | hsa-miR-299-5p |
| 210 | UGUAAACAUCCUACACUCAGCU | hsa-miR-30b |
| 211 | CUGGACUUAGGGUCAGAAGGCC | hsa-miR-422a |
| 212 | CAGCAGCAAUUCAUGUUUUGAA | hsa-miR-424 |
| 213 | AUUUGCUAUCUGAGAGAUGGUGAUGACAUUUUAAACC | RNU24 |
| | ACCAAGAUCGCUGAUGCA | |
| 214 | GUAACUGUGGUGAUGGAAAUGUGUUAGCCUCAGACAC | RNU66 |
| | UACUGAGGUGGUUCUUUCUAUCCUAGUACAGUC | |
| 215 | UUGCACCUCUGAGAGUGGAAUGACUCCUGUGGAGUUG | RNU19 |
| | AUCCUAGUCUGGGUGCAAACAAUU | |
| 216 | CCAGUUCUGCUACUGACAGUAAGUGAAGAUAAAGUGU | RNU38B |
| | GUCUGAGGAGA | |
| 217 | CACUAAUAGGAAGUGCCGUCAGAAGCGAUAACUGACG | RNU49 |
| | AAGACUACUCCUGUCUGAUU | |
| 13 | GAUGACCCCAGGUAACUCUGAGUGUGUCGCUGAUGCC | RNU48 |
| | AUCACCGCAGCGCUCUGACC | |
| 218 | CAUCCCUUGCAUGGUGGAGGGU | hsa-miR-188 |
| 219 | UAAGGUGCAUCUAGUGCAGUUA | hsa-miR-18b |
| 220 | AACUGGCCCUCAAAGUCCCGCUUU | hsa-miR-193b |
| 221 | CAUCUUACCGGACAGUGCUGGA | hsa-miR-200a* |
| 222 | AGAGGUAUAGGGCAUGGGAAAA | hsa-miR-202 |
| 223 | UUUCCUAUGCAUAUACUUCUUU | hsa-miR-202* |
| 224 | CAAAGUGCUCAUAGUGCAGGUAG | hsa-miR-20b |
| 225 | UAUGUGGGAUGGUAAACCGCUU | hsa-miR-299-3p |
| 226 | UAAUGCCCCUAAAAAUCCUUAU | hsa-miR-365 |
| 227 | AGAUCGACCGUGUUAUAUUCGC | hsa-miR-369-5p |
| 228 | AGGUUACCCGAGCAACUUUGCA | hsa-miR-409-5p |
| 229 | ACUUCACCUGGUCCACUAGCCGU | hsa-miR-412 |
| 230 | UAAUACUGUCUGGUAAAACCGU | hsa-miR-429 |
| 231 | UCUUGGAGUAGGUCAUUGGGUGG | hsa-miR-432 |
| 232 | CUGGAUGGCUCCUCCAUGUCU | hsa-miR-432* |
| 233 | AUCAUGAUGGGCUCCUCGGUGU | hsa-miR-433 |
| 234 | UUGCAUAUGUAGGAUGUCCCAU | hsa-miR-448 |
| 235 | UGGCAGUGUAUUGUUAGCUGGU | hsa-miR-449 |
| 236 | UUUUUGCGAUGUGUUCCUAAUA | hsa-miR-450 |
| 237 | UGUUUGCAGAGGAAACUGAGAC | hsa-miR-452 |
| 238 | UCAGUCUCAUCUGCAAAGAAG | hsa-miR-452* |
| 239 | GAGGUUGUCCGUGGUGAGUUCG | hsa-miR-453 |
| 240 | AGAGGCUGGCCGUGAUGAAUUC | hsa-miR-485-5p |
| 241 | CAACCUGGAGGACUCCAUGCUG | hsa-miR-490 |
| 242 | AGUGGGGAACCCUUCCAUGAGGA | hsa-miR-491 |
| 245 | AGGACCUGCGGGACAAGAUUCUU | hsa-miR-492 |
| 246 | UUGUACAUGGUAGGCUUUCAUU | hsa-miR-493 |
| 247 | UGAAACAUACACGGGAAACCUCUU | hsa-miR-494 |
| 248 | AUUACAUGGCCAAUCUC | hsa-miR-496 |
| 249 | CAGCAGCACACUGUGGUUUGU | hsa-miR-497 |
| 250 | UUUCAAGCCAGGGGGCGUUUUUC | hsa-miR-498 |
| 251 | UUAAGACUUGCAGUGAUGUUUAA | hsa-miR-499 |
| 252 | AUGCACCUGGGCAAGGAUUCUG | hsa-miR-500 |
| 253 | AAUCCUUUGUCCCUGGGUGAGA | hsa-miR-501 |
| 254 | UAGCAGCGGGAACAGUUCUGCAG | hsa-miR-503 |
| 255 | GUCAACACUUGCUGGUUUCCUC | hsa-miR-505 |
| 256 | UAAGGCACCCUUCUGAGUAGA | hsa-miR-506 |
| 257 | UUUUGCACCUUUUGGAGUGAA | hsa-miR-507 |
| 258 | UGAUUGUAGCCLTUUUGGAGUAGA | hsa-miR-508 |
| 259 | UGAUUGGUACGUCUGUGGGUAGA | hsa-miR-509 |
| 260 | UGGUAUUGCCAUUGCUUCACUGUUGGCUUUGACCAGG | Z30 |
| | GUAUGAUCUCUUAAUCUUCUCUCUGAGCUG | |
| 261 | CGCAAGGAUGACACGCAAAUUCGUGAAGCGUUCCAUA | RNU6B |
| | UUUUU | |
| 12 | CCUGGAUGAUGAUAGCAAAUGCUGACUGAACAUGAAG | RNU44 |
| | GUCUUAAUUAGCUCUAACUGACU | |
| 263 | GAACUUAUUGACGGGCGGACAGAAACUGUGUGCUGAU | RNU43 |
| | UGUCACGUUCUGAUU | |
| 264 | UCUACAGUGCACGUCUCU | hsa-miR-139 |
| 2 | UGAGAACUGAAUUCCAUAGGCU | hsa-miR-146b-5p |
| 265 | AACAUUCAUUGCUGUCGGUGGG | hsa-miR-181b |
| 266 | AACAUUUCAUUGUUGUCGGUGGGUU | hsa-miR-181d |
| 267 | GGCAAGAUGCUGGCAUAGCUG | hsa-miR-31 |
| 268 | AACACACCUGGUUAACCUCUUU | hsa-miR-329 |
| 269 | AUCAUAGAGGAAAAUCCAUGUU | hsa-miR-376b |
| 270 | AUCGGGAAUGUCGUGUCCGCC | hsa-miR-425 |
| 271 | AAACCGUUACCAUUACUGAGUUU | hsa-miR-451 |
| 272 | CCCAGAUAAUGGCACUCUCAA | hsa-miR-488 |
| 273 | AGUGACAUCACAUAUACGGCAGC | hsa-miR-489 |
| 274 | AAACAAACAUGGUGCACUUCUUU | hsa-miR-495 |
| 275 | AUCCUUGCUAUCUGGGUGCUA | hsa-miR-502 |
| 276 | AGACCCUGGUCUGCACUCUAU | hsa-miR-504 |
| 277 | GUGUCUUUUGCUCUGCAGUCA | hsa-miR-511 |
| 278 | UUCUCCAAAAGAAAGCACUUUCUG | hsa-miR-515-5p |
| 279 | CCUCUAGAUGGAAGCACUGUCU | hsa-miR-517* |
| 280 | AAAGUGCAUCCUUUUAGAGGUUU | bsa-miR-519b |
| 281 | AAAGUGCUUCCUUUUAGAGGG | hsa-miR-520b |
| 282 | AAAGUGCUUCCIJUUUAGAGGGUU | hsa-miR-520c |
| 283 | AAAGUGCUUCUCULTUGGUGGGUU | hsa-miR-520d |
| 284 | AAAGUGCUUCCUULTUUGAGGG | hsa-miR-520e |
| 285 | AAGUGCUUCCUUUUAGAGGGUU | hsa-miR-520f |
| 286 | ACAAAGUGCUUCCCUUUAGAGUGU | hsa-miR-520g |
| 287 | AACGCACUUCCCUUUAGAGUGU | hsa-miR-521 |
| 288 | GAAGGCGCUUCCCUUUAGAGC | hsa-miR-525* |
| 289 | CUCUAGAGGGAAGCACUUUCU | hsa-miR-526a |
| 290 | AAAGUGCUUCCUUUUAGAGGC | hsa-miR-526b* |
| 291 | UACUCAGGAGAGUGGCAAUCACA | hsa-miR-510 |
| 292 | CACUCAGCCUUGAGGGCACUUUC | hsa-miR-512-5p |
| 293 | UUCACAGGGAGGUGUCAUUUAU | hsa-miR-513 |
| 294 | AUUGACACUUCUGUGAGUAG | hsa-miR-514 |
| 295 | GAGUGCCUUCUUUUGGAGCGU | hsa-miR-515-3p |
| 296 | UGCUUCCUUUCAGAGGGU | hsa-miR-516-3p |
| 297 | AUCUGGAGGUAAGAAGCACUUU | hsa-miR-516b |
| 298 | AUCGUGCAUCCCUUUAGAGUGUU | hsa-miR-517 a |
| 299 | UCGUGCAUCCCUUUAGAGUGUU | hsa-miR-517b |
| 300 | AUCGUGCAUCCUUUUAGAGUGU | hsa-miR-517c |
| 301 | AAAGCGCUUCCCUUUGCUGGA | hsa-miR-518a |
| 302 | CAAAGCGCUCCCCUUUAGAGGU | hsa-miR-518b |
| 303 | CAAAGCGCUUCUCUUUAGAGUG | hsa-miR-518e |
| 304 | UCUCUGGAGGGAAGCACUUUCUG | hsa-miR-518c* |
| 305 | CAAAGCGCUUCCCUUUGGAGC | hsa-miR-518d |
| 306 | AAAGCGCUUCCCUUCAGAGUGU | hsa-miR-518e |
| 307 | AAAGCGCUUCUCUUUAGAGGA | hsa-miR-5181 |
| 308 | AAAGUGCAUCCUUUUAGAGUGUUAC | hsa-miR-519a |
| 309 | AAAGUGCAUCUUUUUAGAGGAU | hsa-miR-519c |
| 310 | CAAAGUGCCUCCCUUUAGAGUGU | hsa-miR-519d |
| 311 | AAAGUGCCUCCUUUUAGAGUGU | hsa-miR-519e |
| 312 | UUCUCCAAAAGGGAGCACUUUC | hsa-miR-519e* |
| 313 | AAAGUGCUUCCCUUUGGACUGU | hsa-miR-520a |
| 314 | CUCCAGAGGGAAGUACUUUCU | hsa-miR-520a* |
| 315 | UCUACAAAGGGAAGCCCUUUCUG | hsa-miR-520d* |
| 316 | ACAAAGUGCUUCCCUUUAGAGU | hsa-miR-520h |
| 317 | AAAAUGGUUCCCUUUAGAGUGUU | hsa-miR-522 |
| 318 | AACGCGCUUCCCUAUAGAGGG | hsa-miR-523 |
| 319 | GAAGGCGCUUCCCUUUGGAGU | hsa-miR-524 |
| 320 | CUCCAGAGGGAUGCACUUUCU | hsa-miR-525 |
| 321 | CUCUUGAGGGAAGCACTJUUCUGUU | hsa-miR-526b |
| 322 | CUCUAGAGGGAAGCGCUUUCUGUU | hsa-miR-526c |
| 323 | CUGCAAAGGGAAGCCCUUUCU | hsa-miR-527 |
| 324 | CAGUAGUGAUGAAAUUCCACUUCAUUGGUCCGUGUUU | U18 |
| | CUGAACCACAUGAUUUUCUCGGAUGUUCUGAUG | |
| 325 | CUGCGAUGAUGGCAUUUCUUAGGACACCUUUGGAUUA | RNU58B |
| | AUAAUGAAAACAACUACUCUCUGAGCAGC | |
| 326 | CUGCAGUGAUGACUUUCUUGGGACACCUUUGGAUUUA | RNU58A |
| | CCGUGAAAAUUAAUAAAUUCUGAGCAGC | |
| 327 | CUUAAUGAUGACUGUUUUUUUUGAUUGCUUGAAGCAA | RPL21 |
| | UGUGAAAAACACAUUUCACCGGCUCUGAAAGCU | |
| 328 | UGGCGAUGAGGAGGUACCUAUUGUGUUGAGUAACGGU | U54 |
| | GAUAAUUUUAUACGCUAUUCUGAGCC | |
| 329 | CCAGUCACAGAUUUCUUUGUUCCUUCUCCACUCCCAC | HY3 |
| | UGCAUCACUUAACUAGCCUU | |
| 330 | AGCCUGUGAUGCUUUAAGAGUAGUGGACAGAAGGGAU | U75 |
| | UUCUGAAAUUCUAUUCUGAGGCU | |
| 331 | UAAUGAUUCUGCCAAAUGAAAUAUAAUGAUAUCACUG | U47 |
| | UAAAACCGUUCCAUUUUGAUUCUGAGGU | |
| 332 | AAUUGCACGGUAUCCAUCUGUA | hsa-miR-363 |
| 333 | ACUGCCCUAAGUGCUCCUUCU | hsa-miR-18a* |
| 334 | AAUCCUUGGAACCUAGGUGUGAGU | hsa-miR-362 |
| 335 | AAUAUAACACAGAUGGCCUGU | hsa-miR-410 |
| 336 | UCACUCCUCUCCUCCCGUCUUCU | hsa-miR-483 |
| 337 | GUCAUACACGGCUCUCCUCUCU | hsa-miR-485-3p |
| 338 | UCCUGUACUGAGCUGCCCCGAG | hsa-miR-486 |
| 339 | AAUCAUACAGGGACAUCCAGUU | hsa-miR-487a |
| 340 | UAUGUGCCUUUGGACUACAUCG | hsa-miR-455 |
| 341 | CAUCUGGAGGUAAGAAGCACUUU | hsa-miR-516-5p |
| 342 | UGAAGGUCUACUGUGUGCCAG | hsa-miR-493-3p |
| 343 | CGGGUGGAUCACGAUGCAAUUU | hsa-miR-363* |
| 344 | UGUGACAGAUUGAUAACUGAAA | hsa-miR-542-3p |
| 345 | AAUCGUACAGGGUCAUCCACUU | hsa-miR-487b |
| 346 | GGAGAAAUUAUCCUUGGUGUGU | hsa-miR-539 |
| 347 | GGUAGAUUCUCCUUCUAUGAG | hsa-miR-376a* |
| 348 | UCGGGGAUCAUCAUGUCACGAG | hsa-miR-542-5p |
| 349 | AUCAGCAAACAUUUAUUGUGUG | hsa-miR-545 |
| 350 | AUUCUGCAUUUUUAGCAAGU | hsa-miR-544 |
| 351 | AAUAUUAUACAGUCAACCUCU | hsa-miR-656 |
| 352 | UGACAACUAUGGAUGAGCUCU | hsa-miR-549 |
| 353 | GGCAGGUUCUCACCCUCUCUAGG | hsa-miR-657 |
| 354 | GGCGGAGGGAAGUAGGUCCGUUGGU | hsa-miR-658 |
| 355 | CUUGGUUCAGGGAGGGUCCCCA | hsa-miR-659 |
| 356 | UACCCAUUGCAUAUCGGAGUUG | hsa-miR-660 |
| 357 | AAUGACACGAUCACUCCCGUUGA | hsa-miR-425-5p |
| 358 | AAUGGCGCCACUAGGGUUGUGCA | hsa-miR-652 |
| 359 | CAUGCCUUGAGUGUAGGACCGU | hsa-miR-532 |
| 360 | GCGACCCACUCUUGGUUUCCA | hsa-miR-55 1 a |
| 361 | AACAGGUGACUGGUUAGACAA | hsa-miR-552 |
| 362 | AAAACGGUGAGAUUUUGUUUU | hsa-miR-553 |
| 363 | GCUAGUCCUGACUCAGCCAGU | hsa-miR-554 |
| 364 | AGGGUAAGCUGAACCUCUGAU | hsa-miR-555 |
| 365 | GAUGAGCUCAUUGUAAUAUG | hsa-miR-556 |
| 366 | GUUUGCACGGGUGGGCCUUGUCU | hsa-miR-557 |
| 367 | UGAGCUGCUGUACCAAAAU | hsa-miR-558 |
| 368 | UAAAGUAAAUAUGCACCAAAA | hsa-miR-559 |
| 369 | CAAAGUUUAAGAUCCUUGAAGU | hsa-miR-561 |
| 370 | AAAGUAGCUGUACCAUUUGC | hsa-miR-562 |
| 371 | AGGUUGACAUACGUUUCCC | hsa-miR-563 |
| 372 | AGGCACGGUGUCAGCAGGC | hsa-miR-564 |
| 373 | GGCUGGCUCGCGAUGUCUGUUU | hsa-miR-565 |
| 374 | GGGCGCCUGUGAUCCCAAC | hsa-miR-566 |
| 375 | AGUAUGUUCUUCCAGGACAGAAC | hsa-miR-567 |
| 376 | GCGACCCAUACUUGGUUUCAG | hsa-miR-551b |
| 377 | AGUUAAUGAAUCCUGGAAAGU | hsa-miR-569 |
| 378 | GAAAACAGCAAUUACCUUUGCA | hsa-miR-570 |
| 379 | CAAAACUGGCAAUUACUUUUGC | hsa-miR-548a |
| 380 | UAUGCAUUGUAUUUUUAGGUCC | hsa-miR-586 |
| 381 | UUUCCAUAGGUGAUGAGUCAC | hsa-miR-587 |
| 382 | CAAGAACCUCAGUUGCUUUUGU | hsa-miR-548b |
| 383 | UUGGCCACAAUGGGUUAGAAC | hsa-miR-588 |
| 384 | UCAGAACAAAUGCCGGUUCCCAGA | hsa-miR-589 |
| 385 | UGUCUUACUCCCUCAGGCACAU | hsa-miR-550 |
| 386 | AGACCAUGGGUUCUCAUUGU | hsa-miR-591 |
| 387 | UUGUGUCAAUAUGCGAUGAUGU | hsa-miR-592 |
| 388 | AGGCACCAGCCAGGCAUUGCUCAGC | hsa-miR-593 |
| 389 | CCCAUCUGGGGUGGCCUGUGACUUU | hsa-miR-594 |
| 390 | AAGCCUGCCCGGCUCCUCGGG | hsa-miR-596 |
| 391 | UGUGUCACUCGAUGACCACUGU | hsa-miR-597 |
| 392 | ACAGUCUGCUGAGGUUGGAGC | hsa-miR-622 |
| 393 | GUUGUGUCAGUUUAUCAAAC | hsa-miR-599 |
| 394 | AUCCCUUGCAGGGGCUGUUGGGU | hsa-miR-623 |
| 395 | ACUUACAGACAAGAGCCUUGCUC | hsa-miR-600 |
| 396 | UAGUACCAGUACCUUGUGUUCA | hsa-miR-624 |
| 397 | UGGUCUAGGAUUGUUGGAGGAG | hsa-miR-60 1 |
| 398 | AGCUGUCUGAAAAUGUCUU | hsa-miR-626 |
| 399 | GUGAGUCUCUAAGAAAAGAGGA | hsa-miR-627 |
| 400 | UCUAGUAAGAGUGGCAGUCG | hsa-rniR-628 |
| 401 | GUUCUCCCAACGUAAGCCCAGC | hsa-miR-629 |
| 402 | AGUAUUCUGUACCAGGGAAGGU | hsa-miR-630 |
| 403 | AGACCUGGCCCAGACCUCAGC | hsa-miR-631 |
| 404 | GUGCALTUGCUGUUGCAUUGCA | hsa-miR-33b |
| 405 | CACACACUGCAAUUACUUUUGC | hsa-miR-603 |
| 406 | AGGCUGCGGAAUUCAGGAC | hsa-miR-604 |
| 407 | UAAAUCCCAUGGUGCCUUCUCCU | hsa-miR-605 |
| 408 | AAACUACUGAAAAUCAAAGAU | hsa-miR-606 |
| 409 | GUUCAAAUCCAGAUCUAUAAC | hsa-miR-607 |
| 410 | AGGGGUGGUGUUGGGACAGCUCCGU | hsa-miR-608 |
| 411 | GUGUCUGCUUCCUGUGGGA | hsa- miR-632 |
| 412 | AGGGUGUUUCUCUCAUCUCU | hsa-miR-609 |
| 413 | CUAAUAGUAUCUACCACAAUAAA | hsa-miR-633 |
| 414 | UGAGCUAAAUGUGUGCUGGGA | hsa-miR-610 |
| 415 | AACCAGCACCCCAACUUUGGAC | hsa-miR-634 |
| 416 | ACLTUGGGCACUGAAACAAUGUCC | hsa-miR-635 |
| 417 | GCUGGGCAGGGCUUCUGAGCUCCUU | hsa-miR-612 |
| 418 | UGUGCUUGCUCGUCCCGCCCGCAG | hsa-miR-636 |
| 419 | ACUGGGGGCUUUCGGGCUCUGCGU | hsa-miR-637 |
| 420 | AGGGAUCGCGGGCGGGUGGCGGCCU | hsa-miR-638 |
| 421 | AUCGCUGCGGUUGCGAGCGCUGU | hsa-miR-639 |
| 422 | AUGAUCCAGGAACCUGCCUCU | hsa-miR-640 |
| 423 | AAAGACAUAGGAUAGAGUCACCUC | hsa-miR-641 |
| 424 | AGGAAUGUUCCUUCUUUGCC | hsa-miR-613 |
| 425 | GAACGCCUGUUCUUGCCAGGUGG | hsa-miR-614 |
| 426 | UCCGAGCCUGGGUCUCCCUCU | hsa-miR-615 |
| 427 | ACUCAAAACCCUUCAGUGACUU | hsa-miR-616 |
| 428 | CAAAAAUCUCAAUUACUUUUGC | hsa-miR-548c |
| 429 | AGACUUCCCAUUUGAAGGUGGC | hsa-miR-617 |
| 430 | GUCCCUCUCCAAAUGUGUCUUG | hsa-miR-642 |
| 431 | AAACUCUACUUGUCCUUCUGAGU | hsa-miR-618 |
| 432 | ACUUGUAUGCUAGCUCAGGUAG | hsa-miR-643 |
| 433 | GACCUGGACAUGUUUGUGCCCAGU | hsa-miR-619 |
| 434 | AGUGUGGCUUUCUUAGAGC | hsa-miR-644 |
| 435 | UCUAGGCUGGUACUGCUGA | hsa-miR-645 |
| 436 | GGCUAGCAACAGCGCUUACCU | hsa-miR-621 |
| 437 | AAGCAGCUGCCUCUGAGGC | hsa-miR-646 |
| 438 | GUGGCUGCACUCACUUCCUUC | hsa-miR-647 |
| 439 | AAGUGUGCAGGGCACUGGU | hsa-miR-648 |
| 440 | AAACCUGUGUUGUUCAAGAGUC | hsa-miR-649 |
| 441 | AGGAGGCAGCGCUCUCAGGAC | hsa-miR-650 |
| 442 | UUUAGGAUAAGCUUGACUUUUG | hsa-miR-651 |
| 443 | CAAAAACCACAGUUUCUUUUGC | hsa-miR-548d |
| 444 | UGCCUGGGUCUCUGGCCUGCGCGU | hsa-miR-661 |
| 445 | UCCCACGUUGUGGCCCAGCAG | hsa-miR-662 |
| 446 | AGGCAGUGUAUUGUUAGCUGGC | hsa-miR-449b |
| 447 | UUGAAACAAUCUCUACUGAAC | hsa-miR-653 |
| 448 | UAGUAGACCGUAUAGCGUAC G | hsa-miR-411 |
| 449 | UGGUGGGCCGCAGAACAUGUGC | hsa-miR-654 |
| 450 | AUAAUACAUGGUUAACCUCUUU | hsa-miR-655 |
| 451 | UGAGUUGGCCAUCUGAGUGAG | hsa-miR-571 |
| 452 | GUCCGCUCGGCGGUGGCCCA | hsa-miR-572 |
| 453 | CUGAAGUGAUGUGUAACUGAUCAG | hsa-miR-573 |
| 454 | GAGCCAGUUGGACAGGAGC | hsa-miR-575 |
| 455 | AUUCUAAUUUCUCCACGUCUUUG | hsa-miR-576 |
| 456 | CUUCUUGUGCUCUAGGAUUGU | hsa-miR-578 |
| 457 | AUUCAUUUGGUAUAAACCGCGAU | hsa-miR-579 |
| 458 | UUGAGAAUGAUGAAUCAUUAGG | hsa-miR-580 |
| 459 | UCUUGUGUUCUCUAGAUCAGU | hsa-miR-581 |
| 460 | CAAAGAGGAAGGUCCCAUUAC | hsa-miR-583 |
| 461 | UUAUGGUUUGCCUGGGACUGAG | hsa-miR-584 |
| 462 | UGGGCGUAUCUGUAUGCUA | hsa-miR-585 |
| 463 | UGGCAGUGAUGAUCACAAAUCCGUGUUUCUGACAAGC | U18 |
| | GAUUGACGAUAGAAAACCGGCUGAGCCA | |
| 464 | UAAUACUGCCUGGUAAUGAUGAC | hsa-miR-200b |
| 465 | UCAGGCUCAGUCCCCUCCCGAU | hsa-miR-484 |
| 466 | AAGUGCUGUCAUAGCUGAGGUC | hsa-miR-512-3p |
| 467 | UGUCUUGCAGGCCGUCAUGCA | hsa-miR-431 |
| 468 | CUACAAAGGGAAGCACUUUCUC | hsa-miR-524-5p |
| 469 | UUACAGUUGUUCAACCAGUUACU | hsa-miR-582-5p |
| 470 | GAGCUUAUUCAUAAAAGUGCAG | hsa-miR-590-5p |
| 471 | ACUCCAGCCCCACAGCCUCAGC | hsa-miR-766 |
| 472 | GAAGUGUGCCGUGGUGUGUCU | hsa-miR-595 |
| 473 | UACGUCAUCGUUGUCAUCGUCA | hsa-miR-598 |
| 474 | UUUGUGACCUGGUCCACUAACC | hsa-miR-758 |
| 475 | UGUCACUCGGCUCGGCCCACUAC | hsa-miR-668 |
| 476 | UGCACCAUGGUUGUCUGAGCAUG | hsa-miR-767-Sp |
| 477 | GAUUGCUCUGCGUGCGGAAUCGAC | hsa-miR-801 |
| 478 | UCUGCUCAUACCCCAUGGUUUCU | hsa-miR-767-3p |
| 479 | ACCCUAUCAAUAUUGUCUCUGC | hsa-miR-454* |
| 480 | UGAGACCUCUGGGUUCUGAGCU | hsa-miR-769-5p |
| 481 | GUUGGAGGAUGAAAGUACGGAGUGAU | hsa-miR-768-5p |
| 482 | UCACAAUGCUGACACUCAAACUGCUGAC | hsa-miR-768-3p |
| 483 | UCCAGUACCACGUGUCAGGGCCA | hsa-miR-770-5p |
| 484 | CUGGGAUCUCCGGGGUCUUGGUU | hsa-miR-769-3p |
| 485 | CAGUAACAAAGAUUCAUCCUUGU | hsa-miR-802 |
| 486 | UGGUGCGGAGAGGGCCCACAGUG | hsa-miR-675 |
| 487 | GCACUGAGAUGGGAGUGGUGUA | hsa-miR-674 |
| 488 | AAUGCACCUGGGCAAGGAUUCA | hsa-miR-502-3p |
| 489 | AGACCCUGGUCUGCACUCUAUC | hsa-miR-504 |
| 490 | GUGCAUUGCUGUUGCAUUGC | hsa-miR-33b |
| 491 | GGGAGCCAGGAAGUAUUGAUGU | hsa-miR-505* |
| 492 | UGUGCUUGCUCGUCCCGCCCGCA | hsa-miR-636 |
| 493 | CGUCAACACUUGCUGGUUUCCU | hsa-miR-505 |
| 494 | UUCACAGGGAGGUGUCAU | hsa-miR-513-5p |
| 495 | UAAAUUUCACCUUUCUGAGAAGG | hsa-miR-513-3p |
| 496 | UACUCCAGAGGGCGUCACUCAUG | hsa-miR-508-5p |
| 497 | CGGGGCAGCUCAGUACAGGAU | hsa-miR-486-3p |
| 498 | AUGGUUCCGUCAAGCACCAUGG | hsa-miR-218-1* |
| 499 | AGAGUUGAGUCUGGACGUCCCG | bsa-miR-219-1-3p |
| 500 | ACCUGGCAUACAAUGUAGAUUU | hsa-miR-221 |
| 501 | CUCAGUAGCCAGUGUAGAUCCU | hsa-miR-222* |
| 502 | CGUGUAUUUGACAAGCUGAGUU | hsa-miR-223* |
| 503 | CAAGUCACUAGUGGUUCCGUU | hsa-miR-224 |
| 504 | CAUCAUCGUCUCAAAUGAGUCU | hsa-miR-136* |
| 505 | GAGGGUUGGGUGGAGGCUCUCC | hsa-miR-296-3p |
| 506 | CAAUCACUAACUCCACUGCCAU | hsa-miR-34b |
| 507 | AGGGGCUGGCUUUCCUCUGGUC | hsa-miR-185* |
| 508 | GCCCAAAGGUGAAUUUUUUGGG | hsa-miR-186* |
| 509 | CUCCCACAUGCAGGGUUUGCA | hsa-miR-188-3p |
| 510 | CCAAUAUUGGCUGUGCUGCUCC | hsa-miR-195* |
| 511 | CUGGGAGAGGGUUGUUUACUCC | hsa-miR-30c-1* |
| 512 | UAUUGCACAUUACUAAGUUGCA | hsa-miR-32 |
| 513 | CUGGGAGAAGGCUGUUUACUCU | hsa-miR-30c-2* |
| 514 | CAAUUUAGUGUGUGUGAUAUUU | hsa-miR-32* |
| 515 | UAGCACCAUCUGAAAUCGGUUA | hsa-miR-29a |
| 516 | UGCUAUGCCAACAUAUUGCCAU | hsa-miR-31* |
| 517 | ACUCUUUCCCUGUUGCACUAC | hsa-miR-130b* |
| 518 | CCUAUUCUUGAUUACUUGUUUC | hsa-miR-26a-2* |
| 519 | UCCCCCAGGUGUGAUUCUGAUUU | hsa-miR-361-3p |
| 520 | AACACACCUAUUCAAGGAUUCA | hsa-miR-362-3p |
| 521 | CUGUACAGGCCACUGCCUUGC | hsa-let-7g* |
| 522 | ACUUUAACAUGGAAGUGCUUUC | hsa-miR-302b* |
| 523 | ACUUUAACAUGGAGGCACUUGC | hsa-miR-302d* |
| 524 | ACUGUUGCUAAUAUGCAACUCU | hsa-miR-367* |
| 525 | AACAUAGAGGAAAUUCCACGU | hsa-miR-376c |
| 526 | AAGUGCCGCCAUCUUUUGAGUGU | hsa-miR-371-3p |
| 527 | CUUAUCAGAUUGUAUUGUAAUU | hsa-miR-374a* |
| 528 | UGGGUUCCUGGCAUGCUGAUUU | hsa-miR-23b* |
| 529 | GUAGAUUCUCCUUCUAUGAGUA | hsa-miR-376a* |
| 530 | AGAGGUUGCCCUUGGUGAAUUC | hsa-miR-377* |
| 531 | CUGGGAGGUGGAUGUUUACUUC | hsa-miR-30b* |
| 532 | AACGCCAUUAUCACACUAAAUA | hsa-miR-122* |
| 533 | UUCACAUUGUGCUACUGUCUGC | hsa-miR-130a* |
| 534 | ACCGUGGCUUUCGAUUGUUACU | hsa-miR-132* |
| 535 | UAUGUAACAUGGUCCACUAACU | hsa-miR-379* |
| 536 | AAAGUUCUGAGACACUCCGACU | hsa-miR-148a* |
| 537 | GUGCAUUGUAGUUGCAUUGCA | hsa-miR-33a |
| 538 | CAAUGUUUCCACAGUGCAUCAC | hsa-miR-33a* |
| 539 | AGGUUGGGAUCGGUUGCAAUGCU | hsa-miR-92a-1* |
| 540 | GGGUGGGGAUUUGUUGCAUUAC | hsa-miR-92a-2* |
| 541 | ACUGCUGAGCUAGCACUUCCCG | hsa-miR-93* |
| 542 | AAUCAUGUGCAGUGCCAAUAUG | hsa-miR-96* |
| 543 | CAAGCUCGCUUCUAUGGGUCUG | hsa-miR-99a* |
| 544 | CAAGCUUGUAUCUAUAGGUAUG | hsa-miR-100* |
| 545 | CAGUUAUCACAGUGCUGAUGCU | hsa-miR-101* |
| 546 | GCUAUUUCACGACACCAGGGUU | hsa-miR-138-2* |
| 547 | CAUCUUCCAGUACAGUGUUGGA | hsa-miR-141* |
| 548 | GGUGCAGUGCUGCAUCUCUGGU | hsa-miR-143 * |
| 549 | AGGGGUGCUAUCUGUGAUUGA | hsa-miR-342-5p |
| 550 | GGAUAUCAUCAUAUACUGUAAG | hsa-miR-144* |
| 551 | GGAUUCCUGGAAAUACUGUUCU | hsa-miR-145* |
| 552 | GGGGAGCUGUGGAAGCAGUA | hsa-miR-920 |
| 553 | CUAGUGAGGGACAGAACCAGGAUUC | hsa-miR-921 |
| 554 | GCAGCAGAGAAUAGGACUACGUC | hsa-miR-922 |
| 555 | GUCAGCGGAGGAAAAGAAACU | hsa-miR-923 |
| 556 | AGAGUCUUGUGAUGUCUUGC | hsa-miR-924 |
| 557 | UACUGCAGACGUGGCAAUCAUG | hsa-miR-509-3-5p |
| 558 | GAACGGCUUCAUACAGGAGUU | hsa-miR-337-5p |
| 559 | CUCCUAUAUGAUGCCUUUCUUC | hsa-miR-337-3p |
| 560 | UCACAAGUCAGGCUCUUGGGAC | hsa-miR-125b-2* |
| 561 | AUGUAGGGCUAAAAGCCAUGGG | hsa-rniR-135b* |
| 562 | AAGUUCUGUUAUACACUCAGGC | hsa-miR-148b* |
| 563 | ACUGCCCCAGGUGCUGCUGG | hsa-miR-324-3p |
| 564 | GCUACUUCACAACACCAGGGCC | hsa-miR-138-1* |
| 565 | CCUCUGAAAUUCAGUUCUUCAG | hsa-miR-1460 |
| 566 | AGGGAGGGACGGGGGCUGUGC | hsa-miR-149* |
| 567 | GCUGGUUUCAUAUGGUGGUUUAGA | hsa-miR-29b-1* |
| 568 | CUGGUUUCACAUGGUGGCUUAG | hsa-miR-29b-2* |
| 569 | UCAAAUGCUCAGACUCCUGUGGU | hsa-miR-105 |
| 570 | ACGGAUGUUUGAGCAUGUGCUA | hsa-miR-105* |
| 571 | AAAAGUGCUUACAGUGCAGGUAG | hsa-miR-106a |
| 572 | CUGCAAUGUAAGCACUUCUUAC | hsa-miR- 106a* |
| 573 | CCAAUAUUACUGUGCUGCUUUA | hsa-miR-16-2* |
| 574 | CUGCGCAAGCUACUGCCUUGCU | hsa-let-70 |
| 575 | CGAAUCAUUAUUUGCUGCUCUA | hsa-miR-15b* |
| 576 | AGAGCUUAGCUGAUUGGUGAAC | hsa-miR-27b* |
| 577 | UGUGCGCAGGGAGACCUCUCCC | hsa-miR-933 |
| 578 | UGUCUACUACUGGAGACACUGG | hsa-miR-934 |
| 579 | CCAGUUACCGCUUCCGCUACCGC | hsa-miR-935 |
| 580 | ACAGUAGAGGGAGGAAUCGCAG | hsa-miR-936 |
| 581 | AUCCGCGCUCUGACUCUCUGCC | hsa-miR-937 |
| 582 | UGCCCUUAAAGGUGAACCCAGU | hsa-m111-938 |
| 583 | UGGGGAGCUGAGGCUCUGGGGGUG | hsa-miR-939 |
| 584 | CACCCGGCUGUGUGCACAUGUGC | hsa-miR-941 |
| 585 | UGAGCGCCUCGACGACAGAGCCG | hsa-miR-339-3p |
| 586 | UULTUUCAUUAUUGCUCCUGACC | hsa-miR-335* |
| 587 | GCUGACUCCUAGUCCAGGGCUC | hsa-miR-345 |
| 588 | UCUUCUCUGUUUUGGCCAUGUG | hsa-miR-942 |
| 589 | CUGACUGUUGCCGUCCUCCAG | hsa-miR-943 |
| 590 | AAAUUAUUGUACAUCGGAUGAG | hsa-miR-944 |
| 591 | AGCAGAAGCAGGGAGGUUCUCCCA | hsa-miR-298 |
| 592 | UGCAACGAACCUGAGCCACUGA | hsa-rniR-891a |
| 593 | CGGGUCGGAGLTUAGCUCAAGCGG | hsa-miR-886-5p |
| 594 | CGCGGGUGCUUACUGACCCUU | hsa-miR-886-3p |
| 595 | CACUGUGUCCUUUCUGCGUAG | hsa-miR-892a |
| 596 | CAAGCUCGUGUCUGUGGGUCCG | hsa-miR-99b* |
| 597 | CGUGUUCACAGCGGACCUUGAU | hsa-miR-124* |
| 598 | UCCCUGAGACCCUUUAACCUGUGA | hsa-miR-125a-5p |
| 599 | ACAGGUGAGGUUCUUGGGAGCC | hsa-miR-125a-3p |
| 600 | AAAGGAUUCUGCUGUCGGUCCCACU | hsa-miR-541* |
| 601 | UGGUGGGCACAGAAUCUGGACU | hsa-miR-541 |
| 602 | UUAAUAUCGGACAACCAUUGU | hsa-miR-889 |
| 603 | UAUACCUCAGUUUUAUCAGGUG | hsa-miR-875-5p |
| 604 | CCUGGAAACACUGAGGUUGUG | hsa-miR-875-3p |
| 605 | UGGAUUUCUUUGUGAAUCACCA | hsa-miR-876-5p |
| 606 | CCACCACCGUGUCUGACACUU | hsa-miR-220b |
| 607 | UUUUGCAAUAUGUUCCUGAAUA | hsa-miR-450b-5p |
| 608 | UUGGGAUCAUUUUGCAUCCAUA | hsa-miR-450b-3p |
| 609 | UACUUGGAAAGGCAUCAGUUG | hsa-miR-890 |
| 610 | UGCAACUUACCUGAGUCAUUGA | hsa-miR-891b |
| 611 | ACACAGGGCUGUUGUGAAGACU | hsa-miR-220c |
| 612 | UACUCAAAAAGCUGUCAGUCA | hsa-miR-888 |
| 613 | GACUGACACCUCUUUGGGUGAA | hsa-miR-888* |
| 614 | CACUGGCUCCUUUCUGGGUAGA | hsa-miR-892b |
| 615 | UAGGUAGUUUCCUGUUGUUGGG | hsa-miR-196b |
| 616 | UCACAGUGAACCGGUCUCUUU | hsa-miR-128a |
| 617 | UAAGGUGCAUCUAGUGCAGUUAG | hsa-miR-18b |
| 618 | UACCCUGUAGAACCGAAUUUGUG | hsa-miR-10b |
| 619 | UAAUCUCAGCUGGCAACUGUGA | hsa-miR-216a |
| 620 | UGAGGUAGUAGUUUGUGCUGUU | hsa-let-7i |
| 621 | UGGAAUGUAAAGAAGUAUGUAU | hsa-miR-1 |
| 622 | UGUAAACAUCCUUGACUGGAAG | hsa-miR-30e |
| 623 | UGGUGGUUUACAAAGUAAUUCA | hsa-miR-876-3p |
| 624 | CACAUUACACGGUCGACCUCU | hsa-miR-323-3p |
| 625 | UCGUACCGUGAGUAAUAAUGCG | hsa-miR-126 |
| 626 | CUGAAGCUCAGAGGGCUCUGAU | hsa-miR-127-5p |
| 627 | UCUCUGGGCCUGUGUCUUAGGC | hsa-miR-330-5p |
| 628 | AUAAAGCUAGAUAACCGAAAGU | hsa-miR-9* |
| 629 | UAUAGGGAUUGGAGCCGUGGCG | hsa-miR-135a* |
| 630 | CUAGGUAUGGUCCCAGGGAUCC | hsa-miR-331-5p |
| 631 | UACCACAGGGUAGAACCACGG | hsa-miR-140-3p |
| 632 | UACUGCAGACAGUGGCAAUCA | hsa-miR-509-5p |
| 633 | UGAUUGGUACGUCUGUGGGUAG | hsa-miR-509-3p |
| 634 | AAAAGUAAUUGUGGUUUUUGCC | hsa-miR-548d-5p |
| 635 | UAUGUAACACGGUCCACUAACC | hsa-miR-411* |
| 636 | UAUGUCUGCUGACCAUCACCUU | hsa-miR-654-3p |
| 637 | UCGGGGAUCAUCAUGUCACGAGA | hsa-miR-542-5p |
| 638 | UACUCAGGAGAGUGGCAAUCAC | hsa-miR-510 |
| 639 | ACUGGACUUGGAGUCAGAAGG | hsa-miR-378 |
| 640 | GCAGUCCAUGGGCAUAUACAC | hsa-miR-455-3p |
| 641 | UGGAGUGUGACAAUGGUGUUUG | hsa-miR-122 |
| 642 | UUUGGUCCCCUUCAACCAGCUG | hsa-miR-133a |
| 643 | UUUGGUCCCCUUCAACCAGCUA | hsa-miR-133h |
| 7 | CAUAAAGUAGAAAGCACUACU | hsa-miR-142-5p |
| 645 | UGAGAUGAAGCACUGUAGCUC | hsa-miR-143 |
| 646 | AACUGGCCUACAAAGUCCCAGU | hsa-miR-193a-3p |
| 647 | UAAUACUGCCUGGUAAUGAUGA | hsa-miR-200b |
| 648 | UCCAGCAUCAGUGAUUUUGUUG | hsa-miR-338-3p |
| 649 | UACAGUACUGUGAUAACUGAA | hsa-miR-101 |
| 650 | CUAGACUGAAGCUCCUUGAGG | hsa-miR-151-3p |
| 651 | UCUGGCUCCGUGUCUUCACUCCC | hsa-miR-149 |
| 652 | UCCCUGUCCUCCAGGAGCUCACG | hsa-miR-339-5p |
| 653 | UUAUAAAGCAAUGAGACUGAUU | hsa-miR-340 |
| 654 | UCCGUCUCAGUUACUUUAUAGC | hsa-miR-340* |
| 655 | UCUCACACAGAAAUCGCACCCGU | hsa-miR-342-3p |
| 656 | UAUGGCUUUUCAUUCCUAUGUGA | hsa-miR-135b |
| 657 | GUGUGCGGAAAUGCUUCUGCUA | hsa-miR-147b |
| 658 | UGAUAUGUUUGAUAUUGGGUU | hsa-miR-190b |
| 659 | AAGGUUACUUGUUAGUUCAGG | hsa-miR-872 |
| 660 | AUUCUGCAUUUUUAGCAAGUUC | hsa-miR-544 |
| 661 | UCAGUAAAUGUUUAUUAGAUGA | hsa-miR-545* |
| 662 | UCAGCAAACAUUUAUUGUGUGC | hsa-miR-545 |
| 663 | CUGCCCUGGCCCGAGGGACCGA | hsa-miR-874 |
| 664 | UAUGGCACUGGUAGAAUUCACU | hsa-miR-183 |
| 665 | GUGAAUUACCGAAGGGCCAUAA | hsa-miR-183* |
| 666 | UGGAGAGAAAGGCAGUUCCUGA | hsa-miR-185 |
| 667 | CUGCCAAUUCCAUAGGUCACAG | hsa-miR-192* |
| 668 | GGUCCAGAGGGGAGAUAGGUUC | hsa-miR-198 |
| 669 | CAUCUUACUGGGCAGCAUUGGA | hsa-miR-200b* |
| 670 | GCCUGCUGGGGUGGAACCUGGU | hsa-miR-370 |
| 671 | AGCUACAUCUGGCUACUGGGU | hsa-miR-222 |
| 672 | AAAAGCUGGGUUGAGAGGGCGA | hsa-miR-320 |
| 673 | GUCCAGUUUUCCCAGGAAUCCCU | hsa-miR-145 |
| 674 | AGGCAAGAUGCUGGCAUAGCU | hsa-miR-31 |
| 675 | UGGGUCUUUGCGGGCGAGAUGA | hsa-miR-193a-5p |
| 676 | UGAGGUAGUAGUUUGUACAGUU | hsa-let-7g |
| 677 | AGAGGUAGUAGGUUGCAUAGUU | hsa-let-7d |
| 678 | AGCUGGUGUUGUGAAUCAGGCCG | hsa-miR-138 |
| 679 | CAAAGAAUUCUCCUUUUGGGCU | hsa-rniR-186 |
| 680 | CGUCUUACCCAGCAGUGUUUGG | hsa-miR-200c* |
| 681 | CUCCUACAUAUUAGCAUUAACA | hsa-miR-155* |
| 682 | CAAAUUCGUAUCUAGGGGAAUA | hsa-miR-10a* |
| 683 | UCUACAGUGCACGUGUCUCCAG | hsa-miR-139-5p |
| 684 | AUAAGACGAACAAAAGGUUUGU | hsa-miR-208b |
| 685 | GUGUUGAAACAAUCUCUACUG | hsa-miR-653 |
| 686 | UGCCUGUCUACACUUGCUGUGC | hsa-miR-214* |
| 687 | CAUGGUUCUGUCAAGCACCGCG | hsa-miR-218-2* |
| 11 | UGUCAGUUUGUCAAAUACCCCA | hsa-m IR-223 |
| 689 | UCCAUUACACUACCCUGCCUCU | hsa-miR-885-5p |
| 690 | ACUGGACUUAGGGUCAGAAGGC | hsa-miR-422a |
| 691 | AAGCCCUUACCCCAAAAAGUAU | hsa-miR- 129* |
| 692 | CAACGGAAUCCCAAAAGCAGCUG | hsa-miR-191 |
| 693 | UAAUACUGCCGGGUAAUGAUGGA | hsa-miR-200c |
| 694 | AGUUCUUCAGUGGCAAGCUUUA | hsa-miR-22* |
| 695 | AUCGGGAAUGUCGUGUCCGCCC | hsa-miR-425 * |
| 696 | UUUUGCGAUGUGUUCCUAAUAU | hsa-miR-450a |
| 697 | ACAGUAGUCUGCACAUUGGUUA | hsa-miR-199a-3p |
| 698 | CUUUCAGUCAGAUGUUUGCUGC | hsa-miR-30d* |
| 699 | ACAGCAGGCACAGACAGGCAGU | hsa-miR-214 |
| 700 | CUAUACAAUCUACUGUCUUUC | hsa-let-7a* |
| 10 | CAAAGUGCUUACAGUGCAGGUAG | hsa-miR-17 |
| 701 | CAAAACGUGAGGCGCUGCUAU | hsa-miR-424* |
| 702 | UGCCCUAAAUGCCCCUUCUGGC | hsa-miR-18b* |
| 703 | ACUGUAGUAUGGGCACUUCCAG | hsa-miR-20b* |
| 704 | CAGGUCGUCUUGCAGGGCUUCU | hsa-miR-431* |
| 705 | GGAGACGCGGCCCUGUUGGAGU | hsa-miR-139-3p |
| 706 | CAACAAAUCCCAGUCUACCUAA | hsa-miR-7-2* |
| 707 | ACAGAUUCGAUUCUAGGGGAAU | hsa-miR-10b* |
| 708 | CAAUCAGCAAGUAUACUGCCCU | hsa-miR-34a* |
| 709 | ACCACUGACCGUUGACUGUACC | hsa-miR-181a-2* |
| 710 | AGGUUGUCCGUGGUGAGUUCGCA | hsa-miR-453 |
| 711 | CAUCCCUUGCAUGGUGGAGGG | hsa-miR-188-5p |
| 712 | UCCGGUUCUCAGGGCUCCACC | hsa-rniR-671-3p |
| 713 | UAGUGCAAUAUUGCUUAUAGGGU | hsa-miR-454 |
| 714 | UGCGGGGCUAGGGCUAACAGCA | hsa-miR-744 |
| 715 | CUGUUGCCACUAACCUCAACCU | hsa-miR-744* |
| 716 | AAAUCUCUGCAGGCAAAUGUGA | hsa-miR-216b |
| 717 | UGAGGUUGGUGUACUGUGUGUGA | hsa-miR-672 |
| 718 | CGGCUCUGGGUCUGUCGGGA | hsa-miR-760 |
| 719 | AACUGUUUGCAGAGGAAACUGA | hsa-miR-452 |
| 720 | CUCAUCUGCAAAGAAGUAAGUG | hsa-miR-452* |
| 721 | AGGUUACCCGAGCAACUUUGCAU | hsa-miR-409-5p |
| 722 | GAAUGUUGCUCGGUGAACCCCU | hsa-miR-409-3p |
| 723 | AACCAUCGACCGUUGAGUGGAC | hsa-miR-1810* |
| 724 | UUUGGCAAUGGUAGAACUCACACU | hsa-miR-182 |
| 725 | CGGCAACAAGAAACUGCCUGAG | hsa-miR-196a* |
| 726 | UACUGCAUCAGGAACUGAUUGGA | hsa-miR-217 |
| 727 | AAGACGGGAGGAAAGAAGGGAG | hsa-miR-483-5p |
| 728 | UCACUCCUCUCCUCCCGUCUU | hsa-miR-483-3p |
| 729 | UGAGGGGCAGAGAGCGAGACUUU | hsa-miR-423-5p |
| 730 | AAGGAGCUUACAAUCUAGCUGGG | hsa-miR-708 |
| 731 | CAACUAGACUGUGAGCUUCUAG | hsa-miR-708* |
| 732 | AGGGACGGGACGCGGUGCAGUG | hsa-miR-92b* |
| 733 | GAUGAGCUCAUUGUAAUAUGAG | hsa-miR-556-5p |
| 734 | AUAUUACCAUUAGCUCAUCUUU | hsa-m1R-556-3p |
| 735 | GAAAUCAAGCGUGGGUGAGACC | hsa-miR-551b* |
| 736 | CGAAAACAGCAAUUACCUUUGC | hsa-miR-570 |
| 737 | CACGCUCAUGCACACACCCACA | hsa-miR-574-3p |
| 738 | AUUCUAAUUUCUCCACGUCUUU | hsa-miR-576-5p |
| 739 | AAGAUGUGGAAAAAUUGGAAUC | hsa-miR-576-3p |
| 740 | AAUGGCGCCACUAGGGUUGUG | hsa-miR-652 |
| 741 | GGGGGUCCCCGGUGCUCGGAUC | hsa-miR-615-5p |
| 742 | UAUUCAGAUUAGUGCCAGUCAUG | hsa-miR-871 |
| 743 | CCUCCCACACCCAAGGCUUGCA | hsa-miR-532-3p |
| 744 | GCAGGAACUUGUGAGUCUCCU | hsa-miR-873 |
| 745 | UUGAAAGGCUAUUUCUUGGUC | hsa-miR-488 |
| 746 | GUGACAUCACAUAUACGGCAGC | hsa-miR-489 |
| 747 | CUUAUGCAAGAUUCCCUUCUAC | hsa-miR-491-3p |
| 748 | UGCCCUGUGGACUCAGUUCUGG | hsa-miR-146b-3p |
| 749 | UUCCUAUGCAUAUACUUCUUUG | hsa-miR-202* |
| 750 | AGAGGUAUAGGGCAUGGGAA | hsa-miR-202 |
| 751 | UGAAGGUCUACUGUGUGCCAGG | hsa-miR-493 |
| 752 | UGAAACAUACACGGGAAACCUC | hsa-mR-494 |
| 753 | CGGGGUUUUGAGGGCGAGAUGA | hsa-miR-193b* |
| 754 | AACUGGCCCUCAAAGUCCCGCU | hsa-miR-193b |
| 755 | CAAACCACACUGUGGUGUUAGA | hsa-miR-497* |
| 756 | GAGUGCCUUCUUUUGGAGCGUU | hsa-miR-515-3p |
| 757 | AAGUGCCUCCUUUUAGAGUGUU | hsa-miR-519e |
| 758 | CUCUAGAGGGAAGCGCUUUCUG | hsa-miR-518e* |
| 759 | AGGCAGCGGGGUGUAGUGGAUA | hsa-miR-885-3p |
| 760 | GUGAACGGGCGCCAUCCCGAGG | hsa-miR-887 |
| 761 | AAACAUUCGCGGUGCACUUCUU | hsa-miR-543 |
| 762 | ACGGGUUAGGCUCUUGGGAGCU | hsa-miR-125b-1* |
| 763 | CCAGUGGGGCUGCUGUUAUCUG | hsa-miR-194* |
| 764 | CCGCACUGUGGGUACUUGCUGC | hsa-miR-106b* |
| 765 | ACUUAAACGUGGAUGUACUUGCU | hsa-miR-302a* |
| 766 | CUCUUGAGGGAAGCACUUUCUGU | hsa-miR-526b |
| 767 | GAAAGUGCUUCCUUUUAGAGGC | hsa-miR-526b* |
| 768 | AAAGUGCAUCCUUUUAGAGGUU | hsa-miR-519b-3p |
| 769 | GAAGGCGCUUCCCUUUAGAGCG | hsa-miR-525-3p |
| 770 | GAACGCGCUUCCCUAUAGAGGGU | hsa-miR-523 |
| 771 | CUCUAGAGGGAAGCACUUUCUC | hsa-miR-518f* |
| 772 | GAAAGCGCUUCUCUUUAGAGG | hsa-miR-518f |
| 773 | CUCUAGAGGGAAGCACUUUCUG | hsa-miR-518d-5p |
| 774 | AGAAUUGUGGCUGGACAUCUGU | hsa-miR-219-2-3p |
| 775 | CUUAGCAGGUUGUAUUAUCAUU | hsa-miR-374b* |
| 776 | CAGUGCAAUGAUAUUGUCAAAGC | hsa-miR-30 lb |
| 777 | CUACAAAGGGAAGCCCUUUC | hsa-miR-520d-5p |
| 778 | AAAGCGCUUCCCUUCAGAGUG | hsa-miR-518e |
| 779 | CUGCAAAGGGAAGCCCUUUC | hsa-miR-518a-5p |
| 780 | GAAAGCGCUUCCCUUUGCUGGA | hsa-miR-518a-3p |
| 781 | UUCAUUUGGUAUAAACCGCGAUU | hsa-miR-579 |
| 782 | UAACUGGUUGAACAACUGAACC | hsa-miR-582-3p |
| 783 | AAAGUGCUUCCUUUUAGAGGGU | hsa-miR-520c-3p |
| 784 | CAAAGCGCUUCUCUUUAGAGUGU | hsa-miR-518c |
| 785 | AUCGUGCAUCCCUUUAGAGUGU | hsa-miR-517a |
| 786 | CAAAGUGCCUCCCUUUAGAGUG | hsa-miR-519d |
| 787 | CUAUACAACCUACUGCCUUCCC | hsa-let-7b* |
| 788 | UAGAGUUACACCCUGGGAGUUA | hsa-let-7c* |
| 789 | UGAGGUAGGAGGUUGUAUAGUU | hsa-let-7e |
| 790 | CUAUACGGCCUCCUAGCUUUCC | hsa-let-7e* |
| 791 | AAAAGUAAUUGUGGUUUUGGCC | hsa-miR-548b-5p |
| 792 | UGAGAACCACGUCUGCUCUGAG | hsa-miR-589 |
| 793 | AGUGCCUGAGGGAGUAAGAGCCC | hsa-miR-550 |
| 794 | UGUCUCUGCUGGGGUUUCU | hsa-miR-593 |
| 795 | AAAAGUAAUUGCGAGUUUUACC | hsa-miR-548a-5p |
| 796 | AAAAUGGUUCCCUUUAGAGUGU | hsa-miR-522 |
| 797 | AGUCAUUGGAGGGUUUGAGCAG | hsa-miR-616 |
| 798 | AAAGUGCAUCCUUUUAGAGUGU | hsa-miR-519a |
| 799 | UUCUCGAGGAAAGAAGCACUUUC | hsa-miR-516a-5p |
| 800 | CUAUACAAUCUAUUGCCUUCCC | hsa-let-71-1* |
| 801 | CUAUACAGUCUACUGUCUUUCC | hsa-let-7f-2* |
| 802 | CAGGCCAUAUUGUGCUGCCUCA | hsa-miR-15a* |
| 803 | CCAGUAUUAACUGUGCUGCUGA | hsa-miR-16-1* |
| 804 | ACUGCAGUGAAGGCACUUGUAG | hsa-miR-17* |
| 9 | UAAGGUGCAUCUAGUGCAGAUAG | hsa-miR-18a |
| 805 | ACUGCCCUAAGUGCUCCUUCUGG | hsa-miR-18a* |
| 806 | AGUUUUGCAUAGUUGCACUACA | hsa-miR-19a* |
| 807 | AGUUUUGCAGGUUUGCAUCCAGC | hsa-miR-19b-1* |
| 808 | AGUUUUGCAGGUUUGCAUUUCA | hsa-miR-19b-2* |
| 809 | AACAUCACAGCAAGUCUGUGCU | hsa-miR-499-3p |
| 810 | UAAUCCUUGCUACCUGGGUGAGA | hsa-miR-500 |
| 811 | AAAAGUAAUUGCGGUUUUUGCC | hsa-miR-548c-5p |
| 812 | CACAAGGUAUUGGUAUUACCU | hsa-miR-624 |
| 813 | AGGGGGAAAGUUCUAUAGUCC | hsa-miR-625 |
| 814 | GACUAUAGAACUUUCCCCCUCA | hsa-miR-625* |
| 815 | AUGCUGACAUAUUUACUAGAGG | hsa-miR-628-5p |
| 816 | UCUAGUAAGAGUGGCAGUCGA | hsa-miR-628-3p |
| 817 | AAUGCACCCGGGCAAGGAUUCU | hsa-miR-501-3p |
| 818 | UGGGUUUACGUUGGGAGAACU | hsa-miR-629 |
| 819 | ACUGCAUUAUGAGCACUUAAAG | hsa-miR-20a* |
| 820 | CAACACCAGUCGAUGGGCUGU | hsa-miR-21* |
| 821 | GGGGUUCCUGGGGAUGGGAUUU | hsa-miR-23a* |
| 822 | UGCCUACUGAGCUGAUAUCAGU | hsa-miR-24-1* |
| 823 | UGCCUACUGAGCUGAAACACAG | hsa-miR-24-2* |
| 824 | AGGCGGAGACUUGGGCAAUUG | hsa-miR-25* |
| 825 | CCUAUUCUUGGUUACUUGCACG | hsa-miR-26a-1* |
| 826 | CCUGUUCUCCAUUACUUGGCUC | hsa-miR-26b* |
| 827 | AGGGCUUAGCUGCUUGUGAGCA | hsa-miR-27a* |
| 828 | CACUAGAUUGUGAGCUCCUGGA | hsa-miR-28-3p |
| 829 | ACUGAUUUCUUUUGGUGUUCAG | hsa-miR-29a* |
| 4 | UUAAUGCUAAUCGUGAUAGGGGU | hsa-miR-155 |
| 832 | AGCUCGGUCUGAGGCCCCUCAGU | hsa-miR-423-3p |
| 833 | CUGGUACAGGCCUGGGGGACAG | hsa-miR-150* |
| 834 | UCGAGGAGCUCACAGUCUAGU | hsa-miR-151-5p |
| 835 | UGGAGGAGAAGGAAGGUGAUG | hsa-miR-765 |
| 836 | AACAAUAUCCUGGUGCUGAGUG | hsa-miR-338-5p |
| 837 | AUGGAGAUAGAUAUAGAAAU | hsa-miR-620 |
| 838 | UAGAUAAAAUAUUGGUACCUG | hsa-miR-577 |
| 839 | UACAGUAUAGAUGAUGUACU | hsa-miR-144 |
| 840 | UAAUUUUAUGUAUAAGCUAGU | hsa-miR-590-3p |
| 841 | GCUGCGCUUGGAUUUCGUCCCC | hsa-miR-191* |
| 842 | ACCAGGAGGCUGAGGCCCCU | hsa-miR-665 |
| 843 | AGGUGGUCCGUGGCGCGUUCGC | hsa-miR-323-5p |
| 844 | GGCUACAACACAGGACCCGGGC | hsa-miR-187* |
| 845 | AAAGUGCUUCUCUUUGGUGGGU | hsa-miR-520D-3p |
| 846 | CCCCACCUCCUCUCUCCUCAG | hsa-miR-1224-3p |
| 847 | UCCUCUUCUCCCUCCUCCCAG | hsa-miR-877* |
| 848 | UUCUCAAGGAGGUGUCGUUUAU | hsa-miR-513c |
| 849 | UUCACAAGGAGGUGUCAUUUAU | hsa-miR-513b |
| 850 | GUGAGGGCAUGCAGGCCUGGAUGGGG | hsa-miR-1226* |
| 851 | CCUCUUCCCCUUGUCUCUCCAG | hsa-miR-1236 |
| 852 | GUGUCUGGGCGGACAGCUGC | hsa-miR-1231 |
| 853 | GUGGGCGGGGGCAGGUGUGUG | hsa-miR-1228* |
| 854 | GUGGGUACGGCCCAGUGGGGGG | hsa-miR-1225-5p |
| 855 | UCCUUCUGCUCCGUCCCCCAG | hsa-miR-1237 |
| 856 | UGAGCCCCUGUGCCGCCCCCAG | hsa-miR-1225-3p |
| 857 | UGAGCCCUGUCCUCCCGCAG | hsa-miR-1233 |
| 858 | CGUGCCACCCUUUUCCCCAG | hsa-miR-1227 |
| 859 | UGCAGGACCAAGAUGAGCCCU | hsa-miR-1286 |
| 860 | CAAAGGUAUUUGUGGUUUUUG | hsa-naiR-548m |
| 861 | AAGCAUUCUUUCAUUGGUUGG | hsa-miR-1179 |
| 862 | UUGCUCACUGUUCUUCCCUAG | hsa-miR-1178 |
| 863 | UCUGCAGGGUUUGCUUUGAG | hsa-miR-1205 |
| 864 | CUUGGCACCUAGCAAGCACUCA | hsa-miR-1271 |
| 865 | AGCCUGAUUAAACACAUGCUCUGA | hsa-miR-1201 |
| 866 | GGGCGACAAAGCAAGACUCUULICUU | hsa-miR-1273 |
| 867 | AAAAGUAAUUGCGGUCUUUGGU | hsa-miR-548j |
| 868 | AUGGUACCCUGGCAUACUGAGU | hsa-miR-1263 |
| 869 | UGUGAGGUUGGCAUUGUUGUCU | hsa-miR-1294 |
| 870 | UCAAAACUGAGGGGCAUUUUCU | hsa-miR-1323 |
| 871 | GAUGAUGCUGCUGAUGCUG | hsa-miR-1322 |
| 872 | CUGGACUGAGCCGUGCUACUGG | hsa-miR-1269 |
| 873 | CAGGAUGUGGUCAAGUGUUGUU | hsa-miR-1265 |
| 874 | AAGUAGUUGGUUUGUAUGAGAUGGUU | hsa-miR-1244 |
| 875 | UUUAGAGACGGGGUCUUGCUCU | hsa-miR- 1303 |
| 876 | AUAUAUGAUGACUUAGCUUUU | hsa-miR-1259 |
| 877 | UAAUUGCUUCCAUGUUU | hsa-miR-302f |
| 878 | UAGCAAAAACUGCAGUUACUUU | hsa-miR-548p |
| 879 | CAAGUCUUAUUUGAGCACCUGUU | hsa-miR-1264 |
| 880 | AGAGGAUACCCUUUGUAUGUU | hsa-miR-1185 |
| 881 | CGGAUGAGCAAAGAAAGUGGUU | hsa-miR-1255b |
| 882 | UAAGUGCUUCCAUGCUU | hsa-miR-302e |
| 883 | UCGUUUGCCUUUUUCUGCUU | hsa-miR-1282 |
| 884 | AGGAUGAGCAAAGAAAGUAGAUU | hsa-miR-1255a |
| 885 | CUGGAGAUAUGGAAGAGCUGUGU | hsa-miR-1270 |
| 886 | UAGGACACAUGGUCUACUUCU | hsa-miR-1197 |
| 887 | CAGGGAGGUGAAUGUGAU | hsa-miR-1321 |
| 888 | UGAGGCAGUAGAUUGAAU | hsa-miR-1827 |
| 889 | CCAGACAGAAUUCUAUGCACUUUC | hsa-miR-1324 |
| 890 | AAAAGUAAUCGCGGUUUUUGUC | hsa-miR-548h |
| 891 | AGCCUGGAAGCUGGAGCCUGCAGU | hsa-miR-1254 |
| 892 | AAAAGUACUUGCGGAUUUUGCU | hsa-miR-548k |
| 893 | ACUCUAGCUGCCAAAGGCGCU | hsa-miR-1251 |
| 894 | UCUGGGCAACAAAGUGAGACCU | hsa-miR-1285 |
| 895 | AAGUGAUCUAAAGGCCUACAU | hsa-miR-1245 |
| 896 | UGGGAACGGGUUCCGGCAGACGCUG | hsa-miR-1292 |
| 897 | UCAGCUGGCCCUCAUUUC | hsa-miR-1207-3p |
| 898 | UUGCAGCUGCCUGGGAGUGACUUC | hsa-miR-1301 |
| 899 | UGCUGGAUCAGUGGUUCGAGUC | hsa-miR-1287 |
| 900 | CUCCUGAGCCAUUCUGAGCCUC | bsa-miR-1200 |
| 901 | GAGGGUCUUGGGAGGGAUGUGAC | hsa-miR-1182 |
| 902 | UGGACUGCCCUGAUCUGGAGA | hsa-miR-1288 |
| 903 | UCCCACCGCUGCCACCC | hsa-miR-1280 |
| 904 | UGGCCCUGACUGAAGACCAGCAGU | hsa-miR-1291 |
| 905 | GUGGGGGAGAGGCUGUC | hsa-miR-1275 |
| 906 | CACUGUAGGUGAUGGUGAGAGUGGGCA | hsa-miR-1183 |
| 907 | CCUGCAGCGACUUGAUGGCUUCC | hsa-miR-1184 |
| 908 | UAAAGAGCCCUGUGGAGACA | hsa-miR-1276 |
| 909 | AAAAGCUGGGUUGAGAGGGCAA | hsa-miR-320b |
| 910 | GAUGAUGAUGGCAGCAAAUUCUGAAA | hsa-miR-1272 |
| 911 | UUUCCGGCUCGCGUGGGUGUGU | hsa-miR-1180 |
| 912 | AGGCAUUGACUUCUCACUAGCU | hsa-miR-1256 |
| 913 | UAGUACUGUGCAUAUCAUCUAU | hsa-miR-1278 |
| 914 | AUGGGUGAAUUUGUAGAAGGAU | hsa-miR-1262 |
| 915 | AACUGGAUCAAUUAUAGGAGUG | hsa-miR-1243 |
| 916 | GGUGGCCCGGCCGUGCCUGAGG | hsa-miR-663b |
| 917 | GUGCCAGCUGCAGUGGGGGAG | hsa-miR-1202 |
| 918 | AGAAGGAAAUUGAAUUCAUUUA | hsa-miR-1252 |
| 919 | UUCAUUCGGCUGUCCAGAUGUA | hsa-miR-1298 |
| 920 | UUAGGCCGCAGAUCUGGGUGA | hsa-miR-1295 |
| 921 | UGGAUUUUUGGAUCAGGGA | hsa-miR-1290 |
| 922 | UUUUCAACUCUAAUGGGAGAGA | hsa-miR-1305 |
| 923 | ACGCCCUUCCCCCCCUUCUUCA | hsa-miR-1249 |
| 924 | ACCUUCUUGUAUAAGCACUGUGCUAAA | hsa-miR-1248 |
| 925 | UGGAGUCCAGGAAUCUGCAUUUU | hsa-miR-1289 |
| 926 | UCGUGGCCUGGUCUCCAUUAU | hsa-miR-1204 |
| 927 | AUUGAUCAUCGACACUUCGAACGCAAU | hsa-miR-1826 |
| 928 | UUUGAGGCUACAGUGAGAUGUG | hsa-miR-1304 |
| 929 | GCAUGGGUGGUUCAGUGG | hsa-miR-1308 |
| 930 | CCCGGAGCCAGGAUGCAGCUC | hsa-miR-1203 |
| 931 | UGUUCAUGUAGAUGUUUAAGC | hsa-miR-1206 |
| 932 | AAAACUGUAAUUACUUUUGUAC | hsa-miR-548g |
| 933 | UCACUGUUCAGACAGGCGGA | hsa-miR-1208 |
| 934 | AAAAACUGAGACUACUUUUGCA | hsa-miR-548e |
| 935 | GUCCCUGUUCAGGCGCCA | hsa-miR-1274a |
| 936 | UCCCUGUUCGGGCGCCA | hsa-miR-1274b |
| 937 | CCUGUUGAAGUGUAAUCCCCA | hsa-miR-1267 |
| 938 | ACGGUGCUGGAUGUGGCCUUU | hsa-miR-1250 |
| 939 | CAAAAGUAAUUGUGGAUUUUGU | hsa-miR-548n |
| 940 | UCUACAAAGGAAAGCGCUUUCU | hsa-miR-1283 |
| 941 | ACCCGUCCCGUUCGUCCCCGGA | hsa-miR-1247 |
| 942 | AGAGAAGAAGAUCAGCCUGCA | hsa-miR-1253 |
| 943 | UCUCGCUGGGGCCUCCA | hsa-miR-720 |
| 944 | AUCCCACCUCUGCCACCA | hsa-miR-1260 |
| 945 | UAUUCAUUUAUCCCCAGCCUACA | hsa-miR-664 |
| 946 | UUGGGACAUACUUAUGCUAAA | hsa-miR-1302 |
| 947 | UUGAGAAGGAGGCUGCUG | hsa-miR-1300 |
| 948 | UCUAUACAGACCCUGGCUUUUC | hsa-miR-1284 |
| 949 | AAAAGUAUUUGCGGGUUUUGUC | hsa-miR-5481 |
| 950 | UGGGUGGUCUGGAGAUUUGUGC | hsa-miR-1293 |
| 951 | UCCAGUGCCCUCCUCUCC | hsa-miR-1825 |
| 952 | UUAGGGCCCUGGCUCCAUCUCC | hsa-miR-1296 |
| 953 | AAAAGUAAUUGCGGAUUUUGCC | hsa-miR-548i |
| 954 | AGUGAAUGAUGGGUUCUGACC | hsa-miR-1257 |
| 830 | UCACACCUGCCUCGCCCCCC | hsa-miR-1228 |
| 262 | GACACGGGCGACAGCUGCGGCCC | hsa-miR-602 |
| 102 | CUUCCUCGUCUGUCUGCCCC | hsa-miR-1238 |
| 99 | UAAGGCACGCGGUGAAUGCC | hsa-miR-124-1 |
| 99 | UAAGGCACGCGGUGAAUGCC | hsa-miR-124-2 |
| 99 | UAAGGCACGCGGUGAAUGCC | hsa-miR-124-3 |
| 99 | UAAGGCACGCGGUGAAUGCC | hsa-miR-124b |
| 243 | GCCCCUGGGCCUAUCCUAGAA | hsa-miR-331-3p |
| 244 | AGGGCCCCCCCUCAAUCCUGU | hsa-miR-296-5p |
| 644 | AAUCCUUUGUCCCUGGGUGAGA | hsa-miR-501-5p |
| 955 | UCACAGUGAACCGGUCUCUUU | hsa-miR-128-1 |
| 956 | UCACAGUGAACCGGUCUCUUU | hsa-miR-128-2 |
| 957 | AAGGAGCUCACAGUCUAUUGAG | hsa-miR-28-5p |
| 958 | UGAUUGUAGCCUUUUGGAGUAGA | hsa-miR-508-3p |
| 959 | AGUGGGGAACCCUUCCAUGAGG | hsa-miR-491-5p |
| 960 | AAUCCUUGGAACCUAGGUGUGAGU | hsa-miR-362-5p |
| 961 | UUAUAAUACAACCUGAUAAGUG | hsa-miR-374a |
| 962 | AUAUAAUACAACCUGCUAAGUG | hsa-miR-374b |
| 963 | AUAAUACAACCUGCUAAGUGCU | hsa-miR-374c |
| 964 | CCCAGUGUUCAGACUACCUGUUC | hsa-m1R-199a-1-5p |
| 965 | ACAGUAGUCUGCACAUUGGUUA | hsa-miR-199a-1-3p |
| 966 | CCCAGUGUUCAGACUACCUGUUC | hsa-miR-199a-2-5p |
| 967 | ACAGUAGUCUGCACAUUGGUUA | hsa-miR-199a-2-3p |
| 968 | AUCCUUGCUAUCUGGGUGCUA | hsa-miR-502-5p |
| 969 | AAUGCACCUGGGCAAGGAUUCA | hsa-miR-502-3p |
| 970 | UGGCAGUGUAUUGUUAGCUGGU | hsa-miR-449a |
| 971 | AGGCAGUGUAUUGUUAGCUGGC | hsa-miR-449b |
| 972 | CAGCCACAACUACCCUGCCACU | hsa-miR-449b* |
| 973 | UAGGCAGUGUAUUGCUAGCGGCUGU | hsa-miR-449c |
| 974 | UUGCUAGUUGCACUCCUCUCUGU | hsa-miR-449c* |
| 975 | CUCUAGAGGGAAGCACUUUCUG | hsa-miR-518d-5p |
| 976 | CAAAGCGCUUCCCUUUGGAGC | hsa-miR-518d-3p |
| 977 | UAUGUGCCUUUGGACUACAUCG | hsa-miR-455-5p |
| 978 | GCAGUCCAUGGGCAUAUACAC | hsa-miR-455-3p |
| 979 | UCUCUGGGCCUGUGUCUUAGGC | hsa-miR-330-5p |
| 980 | GCAAAGCACACGGCCUGCAGAGA | hsa-miR-330-3p |
| 981 | CUGAAGCUCAGAGGGCUCUGAU | hsa-miR-127-5p |
| 982 | UCGGAUCCGUCUGAGCUUGGCU | hsa-miR-127-3p |
| 983 | UUAUAAUACAACCUGAUAAGUG | hsa-miR-374a |
| 984 | CUUAUCAGAUUGUAUUGUAAUU | hsa-miR-374a* |
| 985 | AUAUAAUACAACCUGCUAAGUG | hsa-miR-374b |
| 986 | CUUAGCAGGUUGUAUUAUCAUU | hsa-miR-374b* |
| 987 | AUAAUACAACCUGCUAAGUGCU | hsa-miR-374c |
| 988 | CAGUGCAAUAGUAUUGUCAAAGC | hsa-miR-301a |
| 989 | CAGUGCAAUGAUAUUGUCAAAGC | hsa-miR-301b |
| 990 | CAUGCCUUGAGUGUAGGACCGU | hsa-miR-532-5p |
| 991 | CCUCCCACACCCAAGGCUUGCA | hsa-miR-532-3p |
| 992 | UCCUGUACUGAGCUGCCCCGAG | hsa-miR-486-5p |
| 993 | CGGGGCAGCUCAGUACAGGAU | hsa-miR-486-3p |
| 994 | CAGUGGUUUUACCCUAUGGUAG | hsa-miR-140-5p |
| 995 | UACCACAGGGUAGAACCACGG | hsa-miR-140-3p |
| 996 | UGGCAGUGUCUUAGCUGGUUGU | hsa-miR-34a |
| 997 | CAAUCAGCAAGUAUACUGCCCU | hsa-miR-34a* |
| 998 | CAAUCACUAACUCCACUGCCAU | hsa-miR-34b |
| 999 | UAGGCAGUGUCAUUAGCUGAUUG | hsa-miR-34b* |
| 1000 | AGGCAGUGUAGUUAGCUGAUUGC | hsa-miR-34c-5p |
| 688 | AAUCACUAACCACACGGCCAGG | hsa-miR-34c-3p |

*denotes minor sequence as provided by the miRBase database, publicly available at (www.mirbase.org). MiRNAs included in the UPSC miRNA signature are bolded.

The invention provides a microRNA signature comprising hsa-miR-141, hsa-miR-146b-5p, hsa-miR-19a, hsa-miR-155, hsa-miR-142-3p, hsa-miR-24, hsa-miR-142-5p, hsa-miR-19b, hsa-miR-18a, hsa-miR-17-5p, hsa-miR-223, wherein the increased expression of these miRNAs in a cancer cell indicates that the cancer cell originated from a uterine tissue. Alternatively, the microRNA signature consists of hsa-miR-141, hsa-miR-146b-5p, hsa-miR-19a, hsa-miR-155, hsa-miR-142-3p, hsa-miR-24, hsa-miR-142-5p, hsa-miR-19b, hsa-miR-18a, hsa-miR-17-5p, hsa-miR-223, wherein the increased expression of these miRNAs in a cancer cell indicates that the cancer cell originated from a uterine tissue. As such, the miRNA signature is also known as the papillary serous miRNA signature.

More specifically, miR-141 expression is significantly down-regulated in ovarian serous cancer compared to UPSC. Microarray and statistical analyses showed that miR-141 was significantly down-regulated in serous ovarian cancer compared to UPSC. Down-regulation of mir-141, as part of miR-200 family has been described in the epithelial to mesenchymal transition (EMT), essential to cancer progression. Over-expression of miR-141 inhibits EMT and enhances E-cadherin expression, the loss of which is considered as a hallmark of EMT. The difference in miR-141 levels between ovarian and uterine serous cancer and the decrease in the expression levels in ovarian serous carcinoma compared to uterine may be explained by tumor histology. Du et al has recently shown that miR-141 was down-regulated in poorly differentiated or undifferentiated gastric carcinomas cell lines and was up-regulated in well-differentiated gastric tumors (J Gastroenterol. 2009; 44(6):556-61. Epub 2009 Apr. 11).

MiR-146b expression is down-regulated in ovarian serous cancer compared to UPSC. Microarray and statistical analyses showed that miR-146b was also down-regulated in ovarian serous carcinomas compared to uterine tumors. MiR-146a and miR-146b have been shown to inhibit cancer migration and invasion (Bhaumik, D. et al. Oncogene 2008; 42:5643-7). Decreased expression levels of MiR-146a and miR-146b are also consistent with high propensity of ovarian carcinoma to metastasize (Bhaumik, D. et al. Oncogene 2008; 42:5643-7). Herst et al. demonstrated that transduction of miR-146a or miR-146b into the breast cancer cell line, MDA-MB-231, resulted in suppression of metastasis in these cells by 69% to 84% (Hurst, D. R. et al. Cancer Res. 2009 Feb. 15; 69(4):1279-83). MiR-146a and miR-146b gene expression also regulates the body's innate immune response to a variety of microbial components and proinflammatory cytokines (Taganov, K. D. et al. Proc Natl Acad Sci USA 2006 Aug. 15; 103(33): 12481-12486).

MiR-142-3p expression is down-regulated in ovarian serous cancer compared to UPSC. Interestingly, previous studies from our group demonstrated that expression of miR-142-3p is decreased in UPSC compared to better differentiated endometrial tumor subtypes. This miRNA has been found to be associated with bronchoalveolar stem cells. Because UPSC is a more primitive cell type, it may have a larger stem cell component with a unique miRNA signature. The primitive nature of UPSC could explain why miR-142-3p is also low in this tumor. The microarray and statistical analyses of the invention reveal that ovarian serous carcinomas have an even lower expression level of miR-142-3p.

MiR-19a expression is up-regulated in UPSC. Moreover, this miRNA has been identified as a PTEN-targeting miRNA (Pezzolesi, M. G. et al. Am J Hum Genet. 2008 May; 82(5):1141-9). PTEN acts as a tumor suppressor gene through the action of its phosphatase protein product. The PTEN phosphatase is involved in the regulation of the cell cycle, during which it prevents cells from growing and dividing too rapidly. MiR-19a targets PTEN, thereby deregulating the cell cycle.

MiR-155 expression distinguishes uterine from ovarian serous carcinoma. Croce et al has demonstrated miR-155 to play a crucial role in carcinomatogenesis in some types of leukemia and lymphoma (Proc Natl Acad Sci U S A. 2006 Feb. 14; 103(7):2257-61). This group further illustrated that its presence indicated a poorer prognosis in patients with breast and lung cancers. Furthermore, up-regulation of miR-155 has been identified in early pancreatic neoplasia (Habbe, N. et al. Cancer biology & therapy 8(4):340-6, 2009).

MiR-18a expression distinguishes uterine from ovarian serous carcinoma. MiR-18a, included in the signature profile of UPSC-distinguishing miRNAs has been shown to suppress proto-oncogene K-Ras, and, thus, serve as a tumor suppressor (Tsang et al. Carcinogenesis 2009: bgp094v1-bgp094). Tsang et al have demonstrated that miR-18a* repression increased cell proliferation and promoted anchorage-independent growth in human squamous carcinoma A431 cells, colon adenocarcinoma HT-29 cells and fetal hepatic WRL-68 cells. Interestingly, Liu et al. showed that miR-18a was elevated in female patients with hepatocellular carcinoma compared to males (female/male ratio, 4.58; P=0.0023). The gene ESR1 encodes the estrogen receptor-α (ERα), which was identified as a target of miR-18a. Thus, MiR-18a represses ERα translation by binding to its mRNA at the 3′ untranslated region. Furthermore, Liu et al. showed that overexpression of miR-18a decreased ERα levels, thereby stimulating the proliferation of hepatoma cells, which accounted for higher incidences of hepatocellular carcinoma in males than females (Liu et al. Gastroenterology February 2009, Vol. 136, Issue 2, Pages 683-693). High expression of miR-18a has also been correlated with poor prognosis in ovarian cancer (Nam, E. J., Clin Cancer Res 2008 14: 2690-269).

MiR-17, also known as MiR-17-5p, expression is down-regulated in ovarian serous Carcinomas. Mir-17, which was down-regulated in ovarian serous carcinoma compared to UPSC, has been described as a tumor suppressor in breast cancer cells (Hossain, A. et al. Mol Cell Biol. 2006 November; 26(21): 8191-8201). Consequently, expression of miR-17 is low in breast cancer cell lines. Mir-17 downregulates AIB1 resulting in decreased estrogen receptor-mediated, as well as estrogen receptor-independent, gene expression and decreased proliferation of breast cancer cells. AIB1 is a member of the SRC-1 family of non-receptor tyrosine kinases and a steroid receptor coactivator.

MiR-223 distinguishes uterine from ovarian serous carcinomas. Mir-223 has been described as a biomarker of recurrent ovarian cancer (Laios, A. et al. Molecular Cancer 2008, 7:35). MiR-223 was also the most upregulated miRNA in recurrent cancers when compared to primary tumors. Furthermore, miR-223 is highly expressed in cell lines of myeloid origin, suggesting important regulatory roles in human hematopoiesis and oncogenesis. More recently, miR-223 was shown to be a key member of a regulatory circuit that controls granulocytic differentiation and the clinical response of acute promyelocytic leukemia (APL) blasts to all-trans retinoic acid (ATRA). ATRAs appear to be new promising drugs as they have been shown to arrest growth of ovarian carcinoma cells.

### EXAMPLES

### Example 1

### Materials and Methods

### Tissue Collection:

After approval from the Human investigation committee at Yale, uterine and ovarian samples from untreated patients undergoing surgery at Yale New Haven Hospital (New Haven, Conn.) were collected from formalin-fixed paraffin-embedded (FFPE) tissue. All patients underwent staging surgery as initial treatment. No patients receiving neoadjuvant chemotherapy prior to surgery were included. Patient data was collected including age, race, parity and risk factors. All tumors were from primary sites. Preferred primary sites included the uterus or ovary. The carcinoma samples were histologically examined for the presence of tumor. Each sample corresponds to a single patient. A total of 22 UPSC samples and 23 EOC samples were used for analysis.

Fresh/Frozen Preparation: Specimens were immediately snap-frozen and stored at −80° C. All were examined microscopically and microdissected to ensure greater than the preferred 75% tumor cellularity. Specimens may have greater than 50%, 55%, 60%, 65%, 70%, 75%, 80%, 85%, 90%, 95%, 99%, or any percentage point in between of tumor cellularlity.

Paraffin-embedded preparation: Formalin-fixed paraffin-embedded tumors (FFPE) were microdissected and used for microarray analysis. Preferably, sections of tumor have greater than 75% tumor cellularity, however, sections may have greater than 50%, 55%, 60%, 65%, 70%, 75%, 80%, 85%, 90%, 95%, 99%, or any percentage point in between of tumor cellularlity. Twenty-one papillary serous tumors from Yale were identified, microdissected, analyzed by microarray and included in the analysis.

### RNA Extraction

From fresh-frozen tissue: Total RNA isolation, including small RNAs, was performed with the mirVana RNA isolation kit (Ambion, Austin, Tex.) according to the manufacturer's instructions for all fresh frozen tissue. Each sample was derived from a single specimen. Integrity of the RNA was assessed using Nanodrop ND-1000 spectrophotometer (Nanodrop Technologies).

From paraffin-embedded tissue: RNA was extracted from paraffin-embedded slides using Trizol, per protocol. Each sample was derived from a single specimen. Integrity of the RNA was assessed using Nanodrop ND-1000 spectrophotometer (Nanodrop Technologies).

### MiRNA Profiling

cDNA was synthesized from between 160 nanograms (ng) to 800 ng of total RNA using TaqMan MiRNA primers and the TaqMan MiRNA Reverse Transcription Kit (Applied Biosystems). Expression of 384 mature miRNAs was then analyzed with the Asuragen TLDA assay and the Applied Biosystems 7900 Taqman Real-Time PCR machine in accordance with manufacturer's instructions. Fold changes in miRNA expression in different cancer subtypes were determined by delta-delta cycle threshold (CT) values. The cycle threshold value is the number of cycles required for the fluorescent signal to cross the minimal detection threshold (i.e. the signal exceeds background). Normalization was done to two internal small RNA controls RNU44 (encoded by the following nucleic acid sequence: CCUGGAUGAUGAUAGCAAAUGCUGACUGAACAUGAAGGUCUUAAUUAGCUCU AACUGACU, SEQ ID NO: 12) and RNU48 encoded by the following nucleic acid sequence: GAUGACCCCAGGUAACUCUGAGUGUGUCGCUGAUGCCAUCACCGCAGCGCUCU GACC, SEQ ID NO: 13). In the majority of samples, 102 miRNAs were detected from the 384 measured. A CT cutoff of 34 was used in all of the samples. As a confirmation of the data, the first 12 samples were run in duplicate, and the results for each sample when compared between runs were statistically similar.

### Statistical Analysis

Data Normalization: To identify miRNAs whose expression was different between UPSC and EOC, ANOVA analysis was used on normalized data. Samples were normalized to RNU48. Logs of the normalized values were reanalyzed to confirm the findings. P-values were corrected to control for Type I error rates. The intensities were scaled to have similar distributions across the entire series of samples to have the same median absolute deviation across samples. The linear models allowed for general changes in gene expression between different conditions and across different biological replicates. Assessment of differential expression was assessed using a moderated t-statistic. Hierarchical clustering was performed with Pearson correlation and average linkage, based on miRNAs selected for differential expression.

All normalization and data analyses were performed in the statistical programming environment R (www.r-project.org. R Development Core Team: A Language and Environment for Statistical Computing. 2003) and functions available from Bioconductor (Gentleman, R. et al. Genome Biol 2004; 5:R80) and the limma software package.

The sample input CT values were for each miRNA were normalized by quantitating small nuclear RNAs using TaqMan MiRNA Assay Controls (Applied Biosystems). Each of the 8 miRNA reaction pools were normalized separately by the associated small nuclear RNAs. The expression levels of miRNAs within each pool were normalized to a control RNA prior to comparison of the normalized expression levels between pools, which involved a second normalization step. The intensities are scaled to have similar distributions across the entire series of samples to have the same median absolute deviation across samples. The miRNA expression data for different tumor types was analyzed together by using linear modeling methods (Smyth G K. Stat Appl Genet Mol Biol 2004; 3: Article 3.). The linear models allowed for elucidation of general changes in gene expression between different conditions and across different biological replicates. Differential expression was assessed using a moderated t-statistic. P values were adjusted for multiple testing based on all the miRNAs which were expressed in samples (excluding control and unexpressed miRNAs) according to the method of Benjamini and Hochberg (Benjamini Ya Y H. J R Stat Soc B Methodol 1995; 57: 289-300) to control the false discovery rate. Hierarchical clustering was performed with Pearson correlation and average linkage, based on miRNAs selected for differential expression between any of the groups of interest.

Preferred Data Normalization: The sample input CT values were for each miRNA were normalized by quantitating small nuclear RNAs using TaqMan MiRNA Assay Controls (Applied Biosystems). All experimental and control miRNAs were analyzed in a single reaction. The expression levels of experimental miRNAs were normalized to the controls run in the same reaction in a single procedure. This singular normalization preserved differences in expression levels between miRNAs that might have otherwise been minimized by the regular data normalization method. Otherwise, the preferred normalization method is identical to the data normalization method described herein.

### Patient Characteristics

Table 3 describes the clinicopathologic parameters of the study population. Pathologic examination identified primary site of serous tumor as ovary in 23 patients and uterine in 21 patients. The patients' median age was 59 years (range: 43-90) for ovarian carcinoma group and 67 (range: 55-89) for patients with uterine papillary serous carcinoma. In the ovarian cancer group, 21 patients were Caucasian while remaining two were African American and Hispanic. In the UPSC group, 14 patients were Caucasian, 5 African American. Race of the remaining 2 UPSC patients is unknown. Surgical FIGO stage of ovarian cancers was III and IV in 96% of patients. One patient has stage I disease. In the UPSC group, stage III and IV disease accounted for 52% of patients. Remaining patients were diagnosed with stage I and II disease.

TABLE 3
Patient Characteristics

| Clinicopathologic Parameters (n = 44) | |
|---|---|
| Pathology: | |
| Uterine Papillary Serous Carcinoma | 21 |
| Ovarian Serous Carcinoma | 23 |
| Age: | |
| Uterine Papillary Serous Carcinoma | 67 (55-89) |
| Ovarian Serous Carcinoma | 59 (43-90) |
| Race: | |
| Uterine Papillary Serous Carcinoma | |
| Caucasian | 14 |
| African American | 5 |
| Unknown | 2 |
| Ovarian Serous Carcinoma | |
| Caucasian | 21 |
| African American | 1 |
| Hispanic | 1 |
| FIGO Stage | |
| Uterine Papillary Serous Carcinoma | |
| Stage I | 6 |
| Stage II | 4 |
| Stage III | 8 |
| Stage IV | 3 |
| Ovarian Serous Carcinoma | |
| Stage I | 1 |
| Stage II | 0 |
| Stage III | 14 |

### Example 2

### MiRNA Expression Differentiates Uterine from Ovarian Papillary Serous Cancers

Forty-five paraffin-embedded microdissected samples of uterine papillary serous carcinomas and ovarian serous carcinomas were collected from Yale University. MiRNA expression profiles were determined by miRNA profiling analysis followed by statistical analysis.

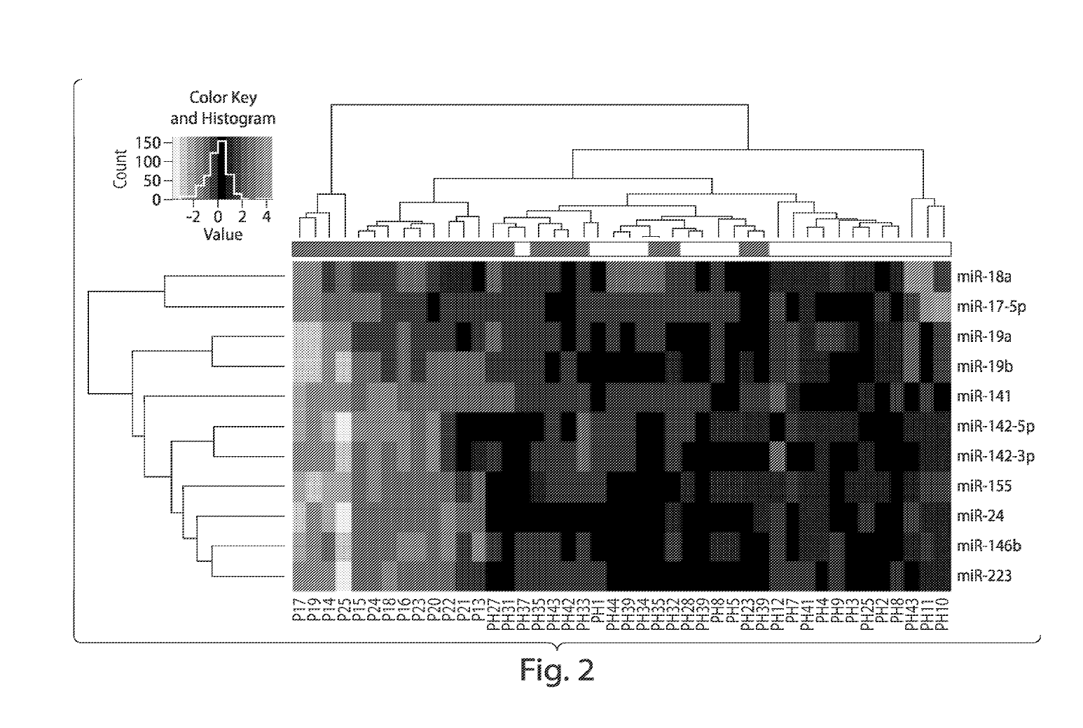

Using the data normalization methods of Example 1, a miRNA expression signature was determined. This signature comprises at least 11 miRNAs that differentiate between uterine and ovarian papillary serous carcinomas (Table 4). When miRNA expression was compared between ovarian serous cancer and uterine papillary serous tumor samples, 8 of the 384 miRNAs showed differential expression with P-values less than 0.05. Another three miRNAs showed differential expression with P-values less than 0.1. Overall, the expression levels of the uterine serous carcinomas are higher than those of ovarian serous tumors. These results are shown graphically in FIG. 2.

TABLE 4
A papillary serous MiRNA Signature

| mlRNA | Sequence * | SEQ ID NO: | Change | P-value |
|---|---|---|---|---|
| hsa-miR-141 | uaacacugucugguaaagaugg | 1 | Higher in UPSC | 0.05 |
| hsa-miR-146b-5p | ugagaacugaauuccauaggcu | 2 | Higher in UPSC | 0.05 |
| hsa-miR-19a | ugugcaaaucuaugcaaaacuga | 3 | Higher in UPSC | 0.05 |
| hsa-miR-155 | uuaaugcuaaucgugauaggggu | 4 | Higher in UPSC | 0.05 |
| hsa-miR-142-3p | uguaguguuuccuacuuuaugga | 5 | Higher in UPSC | 0.05 |
| hsa-miR-24 | uggcucaguucagcaggaacag | 6 | Higher in UPSC | 0.05 |
| hsa-miR-142-5p | cauaaaguagaaagcacuacu | 7 | Higher in UPSC | 0.05 |
| hsa-miR-19b | ugugcaaauccaugcaaaacuga | 8 | Higher in UPSC | 0.05 |
| hsa-miR-18a | uaaggugcaucuagugcagauag | 9 | Higher in UPSC | 0.1 |
| hsa-miR-17 | caaagugcuuacagugcagguag | 10 | Higher in UPSC | 0.1 |
| hsa-miR-223 | ugucaguuugucaaauacccca | 11 | Higher in UPSC | 0.1 |

* Sequences retrieved from the miRBase database, which is publicly available at http://www.mirbase.org/.

### Example 3

### MiRNA Expression Differentiates Synchronous Uterine and Ovarian Papillary Serous Cancers

Fresh and/or frozen, as well as paraffin-embedded, samples of concurrent uterine papillary serous carcinomas and ovarian serous carcinomas were obtained following surgical resection of the tumors of a patient. Importantly, the tumors appeared in both the uterus and the ovary. Moreover, a pathologist could not determine the origin of the tumors using known methods.

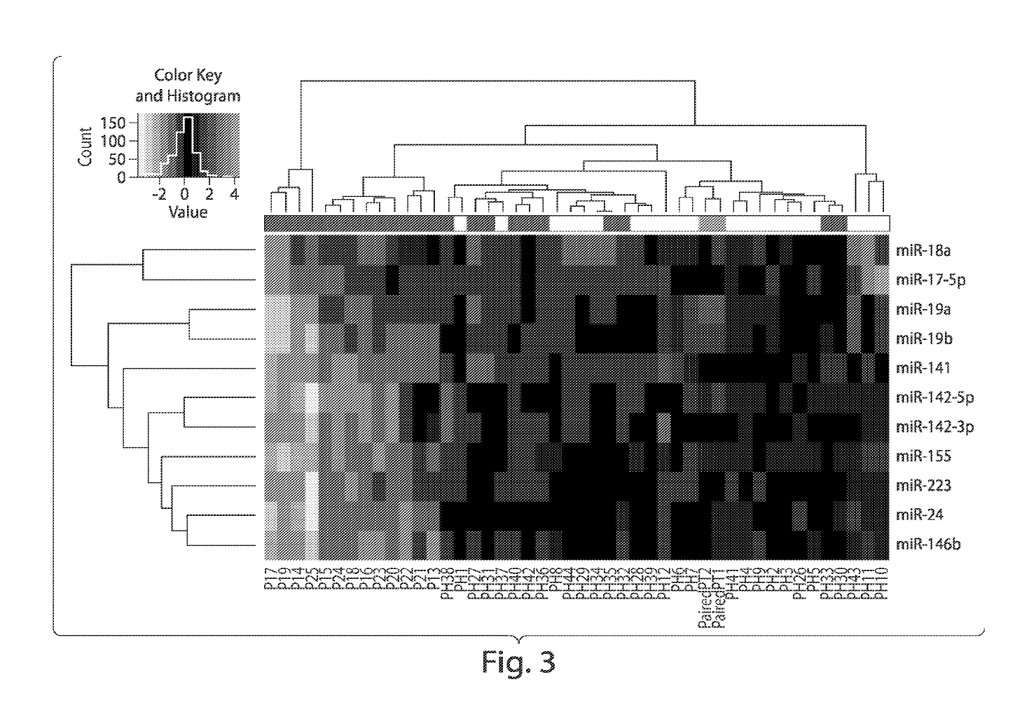

Using the papillary serous miRNA signature of Example 2, the origins of these concurrent uterine papillary serous tumors and ovarian serous tumors were determined. Specifically, the miRNA expression profile of the “unknown” tumors residing in the uterus and ovary, respectively, were determined using the miRNA data and data normalization methods described in Example I. The expression levels of the miRNAs included in the papillary serous miRNA signature of Table 4 were then compared between the “unknown” tumors residing in the uterus and ovary, respectively. The miRNA signatures of the tumors taken from the uterus and the ovary, respectively, were virtually identical. Moreover, the profile was clearly a uterine miRNA profile, as determined by the papillary serous miRNA signature (FIG. 3). Thus, a determination as made that the primary tumor was the uterine tumor, and furthermore, that the uterine tumor had spread into the ovary. The patient was diagnosed with stage III uterine cancer, as opposed to stage I (if the tumors had been synchronous uterine and ovarian cancers) or stage II ovarian cancer. This diagnosis results in a substantially different treatment regime.

This result provides a significant benefit to both a doctor who desires to correctly stage a tumor sample, and to the patient, whose survival and prognosis depends on a correct initial evaluation of the tumor(s).

Synchronous primary cancer is less severe than spread disease. In this example, synchronous primary cancer would have been diagnosed had the tumor obtained from the uterus had a uterine signature and the tumor obtained from the ovary had an ovarian signature. This result would mean that two primary cancers had developed at the same time, or synchronously. However, the discovery that the tumors had the same signature necessarily means that the cancer began in one organ and spread to the other. Because the tumors in this case had a uterine signature, the cancer must have formed in the uterus and spread to the ovary.

The papillary serous miRNA signature described herein is the only method to accurately differentiate between these conditions. This distinction has a profound effect on the diagnosis, prognosis, and treatment of the patient.

### Example 4

### MiRNA Expression Differentiates Spread of Uterine and Ovarian Papillary Serous Cancers

Fresh and/or frozen, as well as paraffin-embedded, samples of uterine papillary serous carcinomas and ovarian serous carcinomas were obtained following surgical resection of tumors from 19 patients. The origins of these tumors were known, however, the miRNA profiles were determined to validate the predictive power of this papillary serous miRNA signature.

Using the miRNAs provided within Table 5, the origins of these uterine papillary serous tumors and ovarian serous tumors were determined. Specifically, the miRNA expression profile of the “blinded” tumors residing in the uterus or ovary, respectively, were determined using the preferred data normalization methods described in Example 1. The expression levels of the miRNAs included in the papillary serous miRNA signature of Table 5 were then compared between the “unknown” tumors residing in the uterus or ovary, respectively.

The preferred data normalization method provides for the validation of a greater number of miRNAs than the standard data normalization method used to generate the first papillary serous signature. Importantly, both signatures differentiate uterine papillary serous carcinomas or ovarian serous carcinomas. As such, both signatures provide clinically relevant and superior information regarding tumor stage and patient diagnosis.

Specifically, Table 5 shows the statistical significance of the change, either by increased or decreased expression, of each miRNA tested between samples of uterine papillary serous carcinomas and ovarian serous carcinomas using the preferred normalization method. Thus, a second papillary serous miRNA signature emerged. Those miRNAs that demonstrate a statistically significant change in expression level between uterine papillary serous carcinomas and ovarian serous carcinomas comprise this papillary serous miRNA signature. A statistically significant change is defined as providing a p-value of less than 0.1, and preferably less than 0.05, and most preferably less than 0.01.

This papillary serous miRNA signature includes hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, or hsa-miR-202.

Optionally, this papillary serous miRNA signature further includes hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, or hsa-miR-484.

Alternatively, this papillary serous miRNA signature further includes. hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, hsa-miR-484, hsa-miR-629, hsa-miR-193b, hsa-miR-885-5p, hsa-miR-155, hsa-miR-200b, hsa-miR-493, hsa-miR-148a, or hsa-miR-101.

In another embodiment, this papillary serous miRNA signature further includes, hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, hsa-miR-484, hsa-miR-629, hsa-miR-193b, hsa-miR-885-5p, hsa-miR-155, hsa-miR-200b, hsa-miR-493, hsa-miR-148a, hsa-miR-101, hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, or hsa-miR-362-5p.

TABLE 5

| miRNA | Raw p-value | Adjusted p-value |
|---|---|---|
| hsa-miR-339-3p | 9.68644E−11 | 2.44098E−08 |
| hsa-miR-548c-5p | 2.38091E−08 | 5.9999E−06 |
| hsa-miR-193a-5p | 4.00415E−07 | 0.000100904 |
| hsa-miR-494 | 2.01259E−06 | 0.000507173 |
| hsa-miR-185 | 4.08968E−06 | 0.001030598 |
| hsa-miR-200c | 4.93376E−06 | 0.001243308 |
| hsa-miR-324-3p | 6.20362E−06 | 0.001563312 |
| hsa-miR-597 | 7.13056E−06 | 0.001796901 |
| hsa-miR-25 | 8.58165E−06 | 0.002162576 |
| hsa-miR-186 | 9.51093E−06 | 0.002396754 |
| hsa-miR-345 | 9.80587E−06 | 0.002471079 |
| hsa-miR-190 | 1.00348E−05 | 0.002528761 |
| hsa-miR-320 | 1.10708E−05 | 0.002789837 |
| hsa-miR-210 | 1.35115E−05 | 0.003404886 |
| hsa-miR-627 | 1.84382E−05 | 0.004646437 |
| hsa-miR-425 | 1.85536E−05 | 0.004675501 |
| hsa-miR-423-5p | 1.97688E−05 | 0.004981729 |
| hsa-miR-636 | 2.1687E−05 | 0.005465135 |
| hsa-miR-141 | 2.30898E−05 | 0.005818622 |
| hsa-miR-125a-5p | 2.65415E−05 | 0.006688448 |
| hsa-miR-342-5p | 2.66013E−05 | 0.006703518 |
| hsa-miR-652 | 2.68562E−05 | 0.006767762 |
| hsa-miR-708 | 2.77136E−05 | 0.00698384 |
| hsa-miR-324-5p | 3.43337E−05 | 0.00865209 |
| hsa-miR-34a | 3.50584E−05 | 0.008834717 |
| hsa-miR-488 | 3.54968E−05 | 0.008945197 |
| hsa-miR-522 | 3.87584E−05 | 0.009767109 |
| hsa-miR-202 | 3.88815E−05 | 0.009798147 |
| hsa-miR-518b | 4.83053E−05 | 0.012172947 |
| hsa-miR-124 | 5.30305E−05 | 0.013363693 |
| hsa-miR-886-3p | 6.08748E−05 | 0.015340449 |
| hsa-miR-361-5p | 6.24935E−05 | 0.015748361 |
| hsa-miR-485-3p | 6.33695E−05 | 0.01596911 |
| hsa-miR-487a | 6.35949E−05 | 0.016025905 |
| hsa-miR-93 | 6.78009E−05 | 0.017085823 |
| hsa-miR-422a | 8.41164E−05 | 0.021197336 |
| hsa-miR-671-3p | 8.65005E−05 | 0.021798124 |
| hsa-miR-625 | 9.16762E−05 | 0.023102407 |
| hsa-miR-142-3p | 0.000101199 | 0.025502045 |
| hsa-miR-331-3p | 0.000113596 | 0.028626306 |
| hsa-miR-512-3p | 0.000124307 | 0.031325462 |
| hsa-miR-92a | 0.000129357 | 0.032597955 |
| hsa-miR-450b-5p | 0.0001462 | 0.036842407 |
| hsa-miR-379 | 0.000146335 | 0.036876378 |
| hsa-miR-29b | 0.000163182 | 0.041121845 |
| hsa-miR-200a | 0.000173887 | 0.043819564 |
| hsa-miR-484 | 0.000180712 | 0.045539541 |
| hsa-miR-629 | 0.000234231 | 0.059026309 |
| hsa-miR-193b | 0.000252005 | 0.063505327 |
| hsa-miR-885-5p | 0.000258364 | 0.065107777 |
| hsa-miR-155 | 0.000287108 | 0.072351274 |
| hsa-miR-200b | 0.000302494 | 0.076228387 |
| hsa-miR-493 | 0.000313392 | 0.078974883 |
| hsa-miR-148a | 0.000376909 | 0.094981055 |
| hsa-miR-101 | 0.000386846 | 0.097485103 |
| hsa-miR-517c | 0.000400896 | 0.101025795 |
| hsa-miR-125a-3p | 0.000406172 | 0.102355226 |
| hsa-miR-9 | 0.000460533 | 0.116054384 |
| hsa-miR-15a | 0.000506616 | 0.12766735 |
| hsa-miR-548d-5p | 0.000506675 | 0.127682159 |
| hsa-miR-579 | 0.000595767 | 0.150133222 |
| hsa-miR-331-5p | 0.000672304 | 0.169420729 |
| hsa-miR-142-5p | 0.000793572 | 0.199980229 |
| hsa-miR-328 | 0.000909184 | 0.229114386 |
| hsa-miR-199b-5p | 0.001205065 | 0.303676474 |
| hsa-miR-135a | 0.001276962 | 0.32179442 |
| hsa-miR-10a | 0.001326249 | 0.334214731 |
| hsa-miR-582-3p | 0.001402897 | 0.353530045 |
| hsa-miR-99b | 0.001488002 | 0.374976493 |
| hsa-miR-487b | 0.001493909 | 0.376464988 |
| hsa-miR-576-3p | 0.001518366 | 0.382628202 |
| hsa-miR-296-5p | 0.001561474 | 0.393491352 |
| hsa-miR-501-5p | 0.001592854 | 0.401399206 |
| hsa-miR-181a | 0.001618255 | 0.407800163 |
| hsa-miR-128 | 0.00173023 | 0.436018001 |
| hsa-miR-483-5p | 0.002105458 | 0.530575324 |
| hsa-miR-28-5p | 0.002316132 | 0.583665288 |
| hsa-miR-299-3p | 0.00232828 | 0.586726442 |
| hsa-miR-505 | 0.002348368 | 0.591788818 |
| hsa-miR-455-3p | 0.002468863 | 0.622153439 |
| hsa-miR-508-3p | 0.002505215 | 0.631314291 |
| hsa-miR-338-3p | 0.002603314 | 0.6560351 |
| hsa-miR-519a | 0.002648593 | 0.667445385 |
| hsa-miR-182 | 0.002866268 | 0.722299428 |
| hsa-miR-500 | 0.003238035 | 0.815984705 |
| hsa-miR-504 | 0.00337887 | 0.851475354 |
| hsa-miR-219-1-3p | 0.003379284 | 0.851579604 |
| hsa-miR-886-5p | 0.003566849 | 0.898846039 |
| hsa-miR-491-5p | 0.003700692 | 0.932574288 |
| hsa-miR-362-5p | 0.003756502 | 0.946638568 |
| hsa-miR-449b | 0.004386287 | 1 |
| hsa-miR-582-5p | 0.004820874 | 1 |
| hsa-miR-187 | 0.005343167 | 1 |
| hsa-miR-429 | 0.005404224 | 1 |
| hsa-miR-570 | 0.005643859 | 1 |
| hsa-miR-136 | 0.005717196 | 1 |
| hsa-miR-193a-3p | 0.005904803 | 1 |
| hsa-miR-598 | 0.005933567 | 1 |
| hsa-miR-374b | 0.006038289 | 1 |
| hsa-miR-28-3p | 0.006119757 | 1 |
| hsa-miR-100 | 0.006515119 | 1 |
| hsa-miR-518e | 0.006719859 | 1 |
| hsa-miR-205 | 0.007108725 | 1 |
| hsa-miR-139-5p | 0.007185918 | 1 |
| hsa-miR-222 | 0.007434382 | 1 |
| hsa-miR-19a | 0.007724305 | 1 |
| hsa-miR-197 | 0.008450396 | 1 |
| hsa-miR-181c | 0.008967617 | 1 |
| hsa-miR-199a-5p | 0.009802495 | 1 |
| hsa-miR-146b-3p | 0.010347544 | 1 |
| hsa-miR-106b | 0.011822793 | 1 |
| hsa-miR-433 | 0.011922418 | 1 |
| hsa-miR-27a | 0.012028875 | 1 |
| hsa-miR-744 | 0.014036622 | 1 |
| hsa-miR-22 | 0.014973478 | 1 |
| hsa-miR-424 | 0.015090141 | 1 |
| hsa-miR-146a | 0.015191404 | 1 |
| hsa-miR-672 | 0.016940591 | 1 |
| hsa-miR-502-5p | 0.018374509 | 1 |
| hsa-miR-523 | 0.018895071 | 1 |
| hsa-miR-511 | 0.020137639 | 1 |
| hsa-miR-23b | 0.020924049 | 1 |
| hsa-miR-132 | 0.021069981 | 1 |
| hsa-miR-449a | 0.021465542 | 1 |
| hsa-miR-375 | 0.022595841 | 1 |
| hsa-miR-518d-3p | 0.024057962 | 1 |
| hsa-miR-224 | 0.024538394 | 1 |
| hsa-miR-495 | 0.024564174 | 1 |
| hsa-miR-299-5p | 0.025072956 | 1 |
| hsa-miR-125b | 0.02602374 | 1 |
| hsa-miR-221 | 0.026851817 | 1 |
| hsa-miR-98 | 0.0319403 | 1 |
| hsa-miR-99a | 0.032576369 | 1 |
| hsa-miR-148b | 0.033091776 | 1 |
| hsa-miR-590-5p | 0.035212093 | 1 |
| hsa-miR-191 | 0.035617382 | 1 |
| hsa-miR-455-5p | 0.036897742 | 1 |
| hsa-miR-330-3p | 0.038342375 | 1 |
| hsa-miR-127-3p | 0.04002545 | 1 |
| hsa-miR-411 | 0.042130554 | 1 |
| hsa-miR-130b | 0.042962772 | 1 |
| hsa-miR-133b | 0.04302539 | 1 |
| hsa-miR-138 | 0.045597505 | 1 |
| hsa-miR-218 | 0.048808583 | 1 |
| hsa-miR-660 | 0.055118613 | 1 |
| hsa-miR-21 | 0.056236454 | 1 |
| hsa-miR-152 | 0.057928712 | 1 |
| hsa-miR-149 | 0.05809225 | 1 |
| hsa-miR-574-3p | 0.06168531 | 1 |
| hsa-let-7f | 0.068122229 | 1 |
| hsa-miR-502-3p | 0.068658472 | 1 |
| hsa-miR-103 | 0.069793814 | 1 |
| hsa-miR-301b | 0.069892091 | 1 |
| hsa-miR-642 | 0.073159016 | 1 |
| hsa-miR-135b | 0.078735247 | 1 |
| hsa-miR-32 | 0.085714743 | 1 |
| hsa-miR-518f | 0.087468875 | 1 |
| hsa-let-7b | 0.089009091 | 1 |
| hsa-miR-29c | 0.090780065 | 1 |
| hsa-miR-203 | 0.092216893 | 1 |
| hsa-miR-10b | 0.100003701 | 1 |
| hsa-miR-363 | 0.10020072 | 1 |
| hsa-miR-192 | 0.10828025 | 1 |
| hsa-miR-362-3p | 0.108489095 | 1 |
| hsa-miR-489 | 0.112547609 | 1 |
| hsa-miR-323-3p | 0.113862151 | 1 |
| hsa-miR-374a | 0.113906633 | 1 |
| hsa-miR-337-5p | 0.1154363 | 1 |
| hsa-miR-451 | 0.123434292 | 1 |
| hsa-miR-301a | 0.1329367 | 1 |
| hsa-miR-509-5p | 0.134439584 | 1 |
| hsa-miR-382 | 0.137422873 | 1 |
| hsa-miR-376a | 0.142538755 | 1 |
| hsa-miR-758 | 0.145352288 | 1 |
| hsa-miR-1 | 0.155384941 | 1 |
| RNU48 | 0.159145683 | 1 |
| hsa-miR-29a | 0.167439823 | 1 |
| hsa-miR-532-5p | 0.168692858 | 1 |
| hsa-miR-365 | 0.169351559 | 1 |
| hsa-miR-27b | 0.186471126 | 1 |
| hsa-miR-184 | 0.196671651 | 1 |
| hsa-miR-133a | 0.198651222 | 1 |
| hsa-miR-450a | 0.201029469 | 1 |
| hsa-miR-34c-5p | 0.214246052 | 1 |
| hsa-miR-96 | 0.228968565 | 1 |
| hsa-miR-214 | 0.238191312 | 1 |
| hsa-miR-18a | 0.245615386 | 1 |
| hsa-miR-618 | 0.252255808 | 1 |
| hsa-miR-146b-5p | 0.275654796 | 1 |
| hsa-miR-486-5p | 0.318463839 | 1 |
| hsa-miR-145 | 0.323567236 | 1 |
| hsa-let-7g | 0.331165061 | 1 |
| hsa-miR-376c | 0.342719699 | 1 |
| hsa-let-7d | 0.34356903 | 1 |
| hsa-miR-199a-3p | 0.344349103 | 1 |
| hsa-miR-130a | 0.371738356 | 1 |
| hsa-miR-532-3p | 0.398426247 | 1 |
| hsa-miR-454 | 0.405013141 | 1 |
| hsa-miR-183 | 0.413877499 | 1 |
| hsa-miR-204 | 0.427769851 | 1 |
| hsa-miR-548b-5p | 0.433185809 | 1 |
| hsa-miR-342-3p | 0.434238114 | 1 |
| hsa-miR-381 | 0.44606785 | 1 |
| hsa-miR-194 | 0.451931403 | 1 |
| hsa-miR-542-3p | 0.453382604 | 1 |
| hsa-miR-20b | 0.476763749 | 1 |
| hsa-miR-539 | 0.478765871 | 1 |
| hsa-miR-223 | 0.483040189 | 1 |
| hsa-miR-140-5p | 0.494598253 | 1 |
| hsa-miR-654-3p | 0.497580566 | 1 |
| hsa-miR-372 | 0.514590779 | 1 |
| RNU44 | 0.525164269 | 1 |
| hsa-miR-24 | 0.531781331 | 1 |
| hsa-miR-410 | 0.537233897 | 1 |
| hsa-miR-31 | 0.549099554 | 1 |
| hsa-miR-196b | 0.552640009 | 1 |
| hsa-miR-15b | 0.577908952 | 1 |
| hsa-miR-486-3p | 0.588963786 | 1 |
| hsa-miR-19b | 0.602063194 | 1 |
| hsa-let-7a | 0.611580561 | 1 |
| hsa-miR-370 | 0.620301194 | 1 |
| hsa-miR-107 | 0.654563147 | 1 |
| hsa-miR-212 | 0.662785982 | 1 |
| hsa-miR-409-5p | 0.66845148 | 1 |
| hsa-miR-542-5p | 0.691778139 | 1 |
| hsa-miR-150 | 0.695426844 | 1 |
| hsa-miR-517a | 0.703503764 | 1 |
| hsa-miR-655 | 0.745473942 | 1 |
| hsa-miR-339-5p | 0.746507063 | 1 |
| hsa-miR-452 | 0.746924511 | 1 |
| hsa-miR-30c | 0.749213087 | 1 |
| hsa-miR-628-5p | 0.753303071 | 1 |
| hsa-miR-195 | 0.762812378 | 1 |
| hsa-miR-143 | 0.777717043 | 1 |
| hsa-miR-16 | 0.783518668 | 1 |
| hsa-miR-26b | 0.784120023 | 1 |
| hsa-miR-545 | 0.804375779 | 1 |
| hsa-miR-335 | 0.841674344 | 1 |
| hsa-miR-17 | 0.851853779 | 1 |
| hsa-miR-30b | 0.868532689 | 1 |
| hsa-miR-18b | 0.874227859 | 1 |
| hsa-miR-134 | 0.875328867 | 1 |
| hsa-miR-503 | 0.880968573 | 1 |
| hsa-miR-340 | 0.892070674 | 1 |
| hsa-let-7c | 0.892798172 | 1 |
| hsa-miR-140-3p | 0.894761477 | 1 |
| hsa-miR-26a | 0.912668645 | 1 |
| hsa-miR-20a | 0.933923082 | 1 |
| hsa-miR-95 | 0.948444924 | 1 |
| hsa-miR-126 | 0.958226092 | 1 |
| hsa-miR-106a | 0.965482613 | 1 |
| hsa-let-7e | 0.968972009 | 1 |

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for determining the origin of a papillary serous carcinoma tumor, the method comprising detecting the miRNA expression profile of a sample from the papillary serous carcinoma tumor and comparing it to an miRNA expression profile of a sample from a uterine tumor or an ovarian tumor, thereby to identify the origin of the papillary serous carcinoma tumor.

2. The method of claim 1, wherein the miRNA expression profile comprises a statistically significant change in the expression of one or more of hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, or hsa-miR-202 in a uterine versus ovarian cancer cell.

3. The method of claim 2, wherein the miRNA expression profile further comprises a statistically significant change in the expression of one or more of one or more of hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, or hsa-miR-484 in a uterine versus ovarian cancer cell.

4. The method of claim 3, wherein the miRNA expression profile further comprises a statistically significant change in the expression of one or more of one or more of hsa-miR-629, hsa-miR-193b, hsa-miR-885-5p, hsa-miR-155, hsa-miR-200b, hsa-miR-493, hsa-miR-148a, or hsa-miR-101 in a uterine versus ovarian cancer cell.

5. The method of claim 4, wherein the miRNA expression profile further comprises a statistically significant change in the expression of one or more of one or more of hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, or hsa-miR-362-5p in a uterine versus ovarian cancer cell.

6. The method of claim 1, wherein the miRNA expression profile comprises the increased expression one or more of hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11) in a uterine versus an ovarian cancer cell.

7. The method of claim 2, wherein the statistically significant change is an increase.

8. The method of claim 2, wherein the statistically significant change is a decrease.

9. A method of determining the origin of a papillary serous carcinoma tumor, comprising the steps of:
(a) obtaining a sample of a papillary serous carcinoma tumor;
(b) extracting total RNA of the sample;
(c) amplifying at least one miRNA from the sample;
(d) determining a miRNA expression profile of the sample; and
(e) comparing the miRNA expression profile of the tumor sample to the papillary serous miRNA signature of claim 31 or 32,
wherein replication of the papillary serous miRNA signature within the miRNA expression profile of the tumor sample indicates that the cells of the tumor sample are uterine cells.

10. The method of claim 9, wherein the papillary serous carcinoma tumor resides in the uterus, ovary, fallopian tube or peritoneum.

11. The method of claim 9, wherein the determining step further comprises normalizing at least one miRNA expression level of at least one miRNA from the tumor sample to a control RNA.

12. The method of 9, wherein the control RNA is RNU44 (SEQ ID NO: 12) or RNU48 (SEQ ID NO: 13).

13. A method of generating a miRNA signature that distinguishes between at least two papillary serous carcinoma tumors of distinct origin, comprising the steps of:
(a) obtaining a sample of at least a first and second papillary serous carcinoma tumor;
(b) extracting total RNA of said first and second samples;
(c) determining a miRNA expression profile of said first and second samples; and
(d) comparing the miRNA expression profiles of said first and second samples,
wherein a plurality of statistically-significant differences identified between the miRNA expression profiles of the first and second miRNA expression profiles identifies a miRNA signature that distinguishes between the first and second papillary serous carcinoma tumors.

14. A method of claim 13, further comprising amplifying at least one miRNA from said first and second samples following the extracting step (b).

15. The method of claim 13, wherein the papillary serous carcinoma tumor resides in the uterus, ovary, fallopian tube, or peritoneum.

16. The method of claim 13, wherein the first or second papillary serous carcinoma tumor is a uterine papillary serous carcinoma tumor.

17. The method of claim 13, wherein the first or second papillary serous carcinoma tumor is an ovarian papillary serous carcinoma tumor.

18. The method of claim 13, wherein the determining step further comprises normalizing at least one miRNA expression level of at least one miRNA from the first or second tumor sample to a control RNA.

19. The method of claim 18, wherein the control RNA is a non-coding RNA selected from the group consisting of transfer RNA (tRNA), small nuclear RNA (snRNA) and small nucleolar RNA (snoRNA).

20. The method of claim 18, wherein the control RNA is a non-coding RNA of between 45 and 200 nucleotides.

21. The method of claim 18, wherein the control RNA is highly- and invariably-expressed between the first and second papillary serous tumor.

22. The method of claim 13, wherein the plurality comprises between 2-30 statistically significant differences.

23. A method of determining the stage of concurrent uterine and ovarian papillary serous carcinoma tumors from a patient, comprising the steps of:
(a) obtaining a sample of a uterine tumor and an ovarian tumor;
(b) extracting total RNA of said uterine sample and said ovarian sample;
(c) determining a miRNA expression profile of the uterine sample and the ovarian sample; and
(d) comparing the miRNA expression profiles of the uterine sample and the ovarian sample to the papillary serous miRNA signature of claim 31 or 32,
wherein replication of the papillary serous miRNA signature within the miRNA expression profile of the uterine sample, but not the ovarian sample, indicates that the uterine and the ovarian tumors are synchronous primary tumors, thereby determining that the tumors are stage I or less.

24. A method of claim 23, further comprising amplifying at least one miRNA from the uterine sample and the ovarian sample following the extracting step (b).

25. A method of determining the stage of concurrent uterine and ovarian papillary serous carcinoma tumors from a patient, comprising the steps of:
(a) obtaining a sample of a uterine tumor and an ovarian tumor;
(b) extracting total RNA of the uterine sample and the ovarian sample;
(c) determining a miRNA expression profile of the uterine sample and the ovarian sample; and
(d) comparing the miRNA expression profiles of the uterine sample and the ovarian sample to the papillary serous miRNA signature of claim 31 or 32,
wherein replication of the papillary serous miRNA signature within the miRNA expression profile of both the uterine and ovarian samples indicates that the uterine tumor is a primary tumor and the ovarian tumor is a metastasis from the uterus, thereby determining that the tumors are at least stage III.

26. The method of claim 25, further comprising amplifying at least one miRNA from the uterine sample and the ovarian sample following the obtaining step (a).

27. A method of determining the stage of concurrent uterine and ovarian papillary serous carcinoma tumors from a patient, comprising the steps of:
(a) obtaining a sample of a uterine tumor and an ovarian tumor;
(b) extracting total RNA of the uterine sample and the ovarian sample;
(c) determining a miRNA expression profile of the uterine sample and the ovarian sample; and
(d) comparing the miRNA expression profiles of the uterine sample and the ovarian sample to the papillary serous miRNA signature of claim 31 or 32,
wherein absence of the papillary serous miRNA signature within the miRNA expression profile of either the uterine and ovarian samples indicates that the ovarian tumor is a primary tumor and the uterine tumor is a metastasis from the ovary, thereby determining that the tumors are at least stage II.

28. The method of claim 27, further comprising amplifying at least one miRNA from the uterine sample and the ovarian sample following the extracting step (b).

29. The method of claim 23, wherein said cancer stage is determined according to the TNM system or the FIGO system.

30. The method of claim 25, wherein said cancer stage is determined according to the TNM system or the FIGO system.

31. A microRNA signature comprising one or more miRNAs selected from the group consisting of hsa-miR-141 (SEQ ID NO: 1), hsa-miR-146b-5p (SEQ ID NO: 2), hsa-miR-19a (SEQ ID NO: 3), hsa-miR-155 (SEQ ID NO: 4), hsa-miR-142-3p (SEQ ID NO: 5), hsa-miR-24 (SEQ ID NO: 6), hsa-miR-142-5p (SEQ ID NO: 7), hsa-miR-19b (SEQ ID NO: 8), hsa-miR-18a (SEQ ID NO: 9), hsa-miR-17 (SEQ ID NO: 10), and hsa-miR-223 (SEQ ID NO: 11), wherein the increased expression of these miRNAs in a uterine versus an ovarian cancer cell indicates that the cancer cell is a uterine cell.

32. A microRNA signature comprising one or more of the miRNAs selected from the group consisting of hsa-miR-339-3p, hsa-miR-548c-5p, hsa-miR-193a-5p, hsa-miR-494, hsa-miR-185, hsa-miR-200c, hsa-miR-324-3p, hsa-miR-597, hsa-miR-25, hsa-miR-186, hsa-miR-345, hsa-miR-190, hsa-miR-320, hsa-miR-210, hsa-miR-627, hsa-miR-425, hsa-miR-423-5p, hsa-miR-636, hsa-miR-141, hsa-miR-125a-5p, hsa-miR-342-5p, hsa-miR-652, hsa-miR-708, hsa-miR-324-5p, hsa-miR-34a, hsa-miR-488, hsa-miR-522, and hsa-miR-202, wherein a statistically significant change in the expression of any one of these miRNAs in a uterine versus ovarian cancer cell indicates that the cancer cell is a uterine cell.

33. The miRNA signature of claim 32, further comprising one or more of the miRNAs selected from the group consisting of hsa-miR-518b, hsa-miR-124, hsa-miR-886-3p, hsa-miR-361-5p, hsa-miR-485-3p, hsa-miR-487a, hsa-miR-93, hsa-miR-422a, hsa-miR-671-3p, hsa-miR-625, hsa-miR-142-3p, hsa-miR-331-3p, hsa-miR-512-3p, hsa-miR-92a, hsa-miR-450b-5p, hsa-miR-379, hsa-miR-29b, hsa-miR-200a, and hsa-miR-484.

34. The miRNA signature of claim 33, further comprising one or more of the miRNAs selected from the group consisting of hsa-miR-629, hsa-miR-193b, hsa-miR-885-5p, hsa-miR-155, hsa-miR-200b, hsa-miR-493, hsa-miR-148a, and hsa-miR-101.

35. The miRNA signature of claim 34, further comprising one or more of the miRNAs selected from the group consisting of hsa-miR-517c, hsa-miR-125a-3p, hsa-miR-9, hsa-miR-15a, hsa-miR-548d-5p, hsa-miR-579, hsa-miR-331-5p, hsa-miR-142-5p, hsa-miR-328, hsa-miR-199b-5p, hsa-miR-135a, hsa-miR-10a, hsa-miR-582-3p, hsa-miR-99b, hsa-miR-487b, hsa-miR-576-3p, hsa-miR-296-5p, hsa-miR-501-5p, hsa-miR-181a, hsa-miR-128, hsa-miR-483-5p, hsa-miR-28-5p, hsa-miR-299-3p, hsa-miR-505, hsa-miR-455-3p, hsa-miR-508-3p, hsa-miR-338-3p, hsa-miR-519a, hsa-miR-182, hsa-miR-500, hsa-miR-504, hsa-miR-219-1-3p, hsa-miR-886-5p, hsa-miR-491-5p, and hsa-miR-362-5p.

36. The miRNA signature of claim 32, wherein the statistically significant change in the expression of any one of these miRNAs is an increase.

37. The miRNA signature of claim 32, wherein the statistically significant change in the expression of any one of these miRNAs is a decrease.

38. The method of claim 27, wherein said cancer stage is determined according to the TNM system or the FIGO system.
